(19) United States
(12) Patent Application Publication
(10) Pub. No.: US 2024/0108721 A1
(43) Pub. Date: Apr. 4, 2024

(54) DUAL TARGETING CHIMERIC ANTIGEN RECEPTORS

(71) Applicant: The University of North Carolina at Chapel Hill, Chapel Hill, NC (US)

(72) Inventors: Gianpietro Dotti, Chapel Hill, NC (US); Hongwei Du, Chapel Hill, NC (US); Koichi Hirabayashi, Nagano (JP); Yang Xu, Shenzhen (CN)

(21) Appl. No.: 18/034,669

(22) PCT Filed: Oct. 29, 2021
(86) PCT No.: PCT/US2021/057407

§ 371 Date: Apr. 28, 2023

Related U.S. Application Data
(60) Provisional application No. 63/108,047, filed on Oct. 30, 2020.

(51) Int. Cl. A61K 39/00; A61P 35/00; C07K 14/705; C07K 14/725; C07K 16/28; C07K 16/30; C12N 5/0783

(52) U.S. Cl. CPC A61K 39/4611; A61K 39/4631; A61K 39/464411; A61K 39/464471; A61P 35/00; C07K 14/7051; C07K 14/70517; C07K 14/70521; C07K 14/70578; C07K 16/2827; C07K 16/3084; C12N 5/0636; A61K 2239/22; A61K 2239/28; C07K 2317/622; C07K 2319/03

(57) ABSTRACT

Disclosed herein is a chimeric antigen receptor T cell therapy for treating patients having a cancer, such as a cancer having one or more solid tumors.

### CROSS-REFERENCE TO RELATED APPLICATIONS

This application claims the benefit of U.S. Provisional Application No. 63/108,047, filed on Oct. 30, 2020. The entire teachings of the above application are incorporated herein by reference.

### GOVERNMENT SUPPORT

This invention was made with government support under Grant Numbers CA193140 and CA243543 awarded by the National Institutes of Health. The government has certain rights in the invention.

### BACKGROUND OF THE INVENTION

The development of successful CAR-T cells in solid tumors poses critical issues that include selection of the appropriate targets to prevent on-target, but off-tumor toxicity, simultaneous recognition of multiple targets to prevent tumor escape and protection from the immune suppressive tumor microenvironment^{1,2} Several CARs targeting antigens in solid tumors are currently under clinical investigation with the primary endpoint to establish safety of the selected antigen target¹. However, the construction of the next generation CARs targeting simultaneously at least two antigens as well as the definition of the most appropriate way to accommodate the intracytoplasmic domains of the CARs remains challenging. Single CAR cassettes with dual targeting have been generated by fusing antigen binding moieties to one single CAR stem that provides co-stimulation and CD3ζ signaling³⁻⁷. The main disadvantage of this design is the difficulty in maintaining the structural integrity of the assembled antigen binding moieties that have an intrinsic propensity to unfold⁸. Furthermore, while T cell co-stimulation provided by either CD28 or 4-1BB endodomains are equally effective in promoting clinical remission in patients with B-cell malignancies^{9,10}, there is a common perception in the field that dual CD28 and 4-1BB co-stimulation may promote rapid tumor regression sustained by CD28 and long-term persistence provided by 4-1BB^{9,11,12}.

Optimal T cell co-stimulation is the first critical event to counter immunosuppression within the tumor microenvironment of solid tumors. Multiple co-stimulation in CAR-T cells has been achieved by either inclusion in tandem of two or three co-stimulatory endodomains (3^{rd} generation CARs) or by supplying 4-1BB ligand to CAR-T cells that encode CD28¹³⁻¹⁶. However, reported clinical data did not demonstrate a significant advantage in term of objective clinical responses of 3^{rd} generation CAR-T cells, suggesting that in cis co-stimulatory endodomains may not provide the spatial distribution of CD28 and 4-1BB co-stimulation required to promote optimal T cell activation and survival.^{15,17,18}.

### SUMMARY OF THE INVENTION

Disclosed herein is an approach based on dual targeting, split co-stimulatory signaling and shared CD3ζ chain tailored to target two clinically relevant antigens-GD2 and B7-H3 —in the disease model of neuroblastoma¹⁹⁻²¹. It was demonstrated that this design strategy achieves rapid and sustained antitumor effects, which are sustained by optimized signaling, effector molecular signature and metabolic fitness of the CAR-T cells. Furthermore, dual antigen targeting prevents tumor escape due to heterogeneity of antigen expression in tumor cells.

Disclosed herein are modified T cells; in some embodiments the modified T cell includes a dual targeting CAR with split costimulatory signal and a single CAR-CD3ζ domain.

In some embodiments, the T cell co-stimulates CD28 and 4-1BB. In some embodiments, the T cell expresses GD2 and B7-H3.

In some embodiments, the T cell exhibits dual antigen specificity and co-stimulation. In some embodiments, the T cell exhibits killing activity and cytokine release of T cells via the GD2.28ζ.CAR or B7-H3.BB.CAR. In some embodiments, the T cell exhibits increased IFN-γ and IL-2 release, as compared to a control cell. In some embodiments, the T cell exhibits higher basal levels of TCR activation signaling, as compared to a control cell. In some embodiments, the T cell exhibits enhanced phosphorylation of the CAR-CD3ζ chain and downstream signaling kinases, which may include ERK and Akt.

In some embodiments, the T cell exhibits enrichment in cell cycle pathways, e.g., at 5 days upon removal from antigen stimulation. In some embodiments, the T cell exhibits enrichment in TCR signaling pathways, e.g., at 5 days upon removal from antigen stimulation. In some embodiments, the T cell exhibits elevated glycolytic activity, as compared to a control cell, e.g., at day 0 and day 5 post-stimulation.

In some embodiments, the T cell controls tumor growth upon tumor re-challenge, as compared to a control cell. In some embodiments, the T cell promotes enhanced tumor control and improved survival, as compared to a control cell. In some embodiments, the T cell exhibits increased anti-tumor activity. In some embodiments, the T cell exhibits increased anti-tumor activity under stress conditions.

In some embodiments, the T cell is a human T cell. In some embodiments, the T cell is a non-human T cell. In some embodiments, the T cell is a mouse T cell.

Also disclosed herein are methods of treating cancer. The methods include administering to a subject a modified T cell comprising a dual targeting CAR with split costimulatory signal and a single CAR-CD3ζ domain.

In some embodiments, the T cell costimulates CD28 and 4-1BB. In some embodiments, the T cell expresses GD2 and B7-H3. In some embodiments, the modified T cell exhibits increased anti-tumor activity. In some embodiments, the modified T cell protects from tumor re-challenge.

In some embodiments, the cancer is a neuroblastoma. In some embodiments, the subject is a mammal. In some embodiments, the subject is human.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1H demonstrate single or dual antigen targeting and single or dual CD28 or 4-1BB co-stimulation do not eradicate the tumor in stress conditions. FIG. 1A shows schema of the CHLA-255 metastatic xenograft NB model in NSG mice inoculated intravenously via tail vein with FFLuc-labelled CHLA-255 cells and treated 14 days later with low doses of CAR-T cells targeting either GD2 or B7-H3. FIGS. 1B-1C provide representative tumor bioluminescence (BLI) images (FIG. 1B) and BLI kinetics (FIG. 1C) of FFLuc-CHLA-255 tumor growth in the metastatic xenograft NB model shown in FIG. 1A (n=5 mice/group). FIG. 1D provides a Kaplan-Meier survival curve of mice in FIGS. 1B-1C (n=5 mice/group); **p<0.01 by chi-square test. FIG. 1E shows schema of the CHLA-255 metastatic xenograft NB model in NSG mice inoculated intravenously via tail vein with FFLuc-labelled CHLA-255 cells and treated 14 days later received low doses of CAR-T cells targeting either GD2 or both GD2 and B7-H3. FIGS. 1F-1G provides representative tumor BLI (FIG. 1F) and BLI kinetics (FIG. 1G) of FFLuc-CHLA-255 tumor growth in the metastatic xenograft NB models shown in FIG. 1E (n=4 or 5 mice/group). FIG. 1H provides Kaplan-Meier survival curve of mice in FIGS. 1F-1G (n=4 or 5 mice/group); **p<0.01 by chi-square test.

FIGS. 2A-2Q demonstrate dual targeting, split signaling and one single CD3ζ endodomain promote sustained T cell activation profile without inducing T cell exhaustion. FIG. 2A provides representative flow cytometry histograms showing the expression of CARs in human T cells transduced with retroviral vectors encoding the CD19.28ζ.CAR, GD2.28ζ.CAR, or GD2.28ζ.CAR/B7-H3.BB.CAR. FIG. 2B provides a summary of the CD19.28ζ.CAR, GD2.28ζ.CAR, or GD2.28ζ.CAR/B7-H3.BB.CAR transduction efficiency (n=13). The horizontal bars represent the mean values. FIG. 2C shows schema of the repetitive co-culture experiment of CAR-T cells and NB cell lines. Tumor cells were seeded in 24-well plates one day prior to the addition of T cells. At day 0, CAR-T cells were added at T cell to tumor cell ratio of 1 to 5. At day 4, 6, and 8, all T cells were collected and transferred into a new well in which 5×10⁵ NB cells were seeded one day prior to the addition of T cells. T cells and NB cells were quantified by flow cytometry after each cycle. Supernatant were also collected for cytokine measurements 24 hours after adding T cells for each cycle.

FIGS. 2D-2E show quantification of residual CHLA-255 cells (FIG. 2D) and enumeration of T cells (FIG. 2E) in the repetitive co-cultures. Error bars denote SE, n=12; ***p<0.001, ns (not significant) by one-way ANOVA with Tukey's multiple comparison test adjusted p value. FIGS. 2F-2G provide a summary of IFN-γ (FIG. 2F) and IL-2 (FIG. 2G) released by CAR-T cells in the culture supernatant after 24 hours of co-culture with CHLA-255 cells as measured by ELISA. Error bars denote SE, n=12; ***p<0.001 by one-way ANOVA with Tukey's multiple comparison test adjusted p value. FIGS. 2H-2I provide quantification of residual LAN-1 cells (FIG. 2H) and enumeration of T cells (FIG. 2I) in the repetitive co-culture experiments. Error bars denote SE, n=12; **p<0.01, ***p<0.001, ns (not significant) by one-way ANOVA with Tukey's multiple comparison test adjusted p value. FIGS. 2J-2K provide a summary of IFN-γ (FIG. 2J) and IL-2 (FIG. 2K) released by CAR-T cells in the culture supernatant after 24 hours of co-culture with LAN-1 cells as measured by ELISA. Error bars denote SE, n=12; **p<0.01, ***p<0.001 by one-way ANOVA with Tukey's multiple comparison test adjusted p value. FIG. 2L shows schema of the CHLA-255 metastatic xenograft NB model in NSG mice inoculated intravenously via tail vein with FFLuc-labelled CHLA-255 cells and treated 14 days later with low doses of CAR-T cells targeting either GD2 or GD2 and B7-H3. FIGS. 2M-2N provide representative tumor BLI images (FIG. 2M) and BLI kinetics (FIG. 2N) of FFLuc-CHLA-255 tumor growth in the metastatic xenograft NB models shown in FIG. 2L (n=5 mice/group). FIG. 2O shows Kaplan-Meier survival curve of mice in (FIGS. 2M-2N) (n=5 mice/group); **p<0.01 by chi-square test. FIGS. 2P-2Q show detection of circulating CAR-T cells (CD45+CD3+) in mice 14 days (FIG. 2P) and 28 days (FIG. 2Q) after CAR-T cell treatment by flow cytometry (n=3-5 mice/group); *p<0.05, ns (not significant) by one-way ANOVA with Tukey's multiple comparison test adjusted p value or unpaired t test.

FIGS. 3A-3M demonstrate dual targeting, split signaling and one single CD3ζ endodomain promote TCR tonic signaling and both glycolytic and oxidative metabolism. FIG. 3A show RNAseq analysis of non stimulated CAR-T cells. FIGS. 3B-3D provide gene set enrichment analysis (GSEA) of glycolytic (FIG. 3B), IFN-γ signaling pathways (FIG. 3C), and TCR upregulated genes (FIG. 3D) in non stimulated T cells expressing GD2.28ζ.CAR or GD2.28ζ.CAR/B7-H3.BB.CAR.

FIGS. 3E-3H show RNAseq analysis of GD2.28ζ.CAR-T cells and GD2.28ζ.CAR/B7-H3.BB.CAR-T cells at one day (FIG. 3E) and five days (FIG. 3F) after CAR stimulation. FIGS. 3G-3H show GSEA of the cell cycle (FIG. 3G) and TCR (FIG. 3H) signaling five days after CAR stimulation. FIG. 3I shows principal component analysis of transcriptome data from CAR-T cells at day 0, 1 and 5. FIGS. 3J-3K show proliferation of the CAR-T cells after CAR stimulation. FIG. 3J shows CAR-T cells were stained with CFSE and then stimulated via 1A7 Ab and B7-H3-Fc protein on day 0, the CFSE dilution were examined by flow cytometry on day 3 and day 6 after stimulation, and the cell numbers (FIG. 3K) were counted by flow cytometry with counting beads in a separate experiment without CFSE staining (n=5, *p=0.0283). FIGS. 3L-3M provide metabolic profile showing glucose (FIG. 3L) and O₂ consumption (FIG. 3M) of GD2.28ζ.CAR-T cells and GD2.28ζ.CAR/B7-H3BB.CAR-T cells before CAR activation (resting), and day 1 and day 5 after CAR activation. Extracellular acidification rate (ECAR) and oxygen consumption rate (OCR) were assayed in different time points in a Seahorse XF24 analyzer (n=3, *p<0.05, **p<0.01, ***p<0.001,****p<0.0001). The long and short arrows indicate the time point of adding Rot/AA and 2-DG respectively (FIG. 3L); the black, green and purple arrows indicate the time point of adding oligomycin, FCCP, Rot/AA respectively (FIG. 3M).

FIGS. 4A-4I demonstrate dual targeting, split signaling and one single CD3ζ endodomain prevent tumor escape due to antigen loss. FIG. 4A provides flow cytometry histogram showing the expression of GD2 and B7-H3 in a human NB cell line SH-SY5Y stained. FIG. 4B shows quantification of residual NB cells labelled with GFP co-cultured with CAR-T cells at the T cell to tumor cell ratio of 1 to 5. On day 5, NB cells (GFP⁺) and CAR-T cells (CD3⁺) were enumerated by flow cytometry. Error bars denote SE, n=10; *p<0.05, ***p<0.001 by one-way ANOVA with Tukey's multiple comparison test adjusted p value. FIGS. 4C-4D show summary of IFN-γ (FIG. 4C) and IL-2 (FIG. 4D) released by CAR-T cells in the culture supernatant after 24 hours of co-culture with NB cells as measured by ELISA. Error bars denote SE, n=10; *p<0.05, **p<0.01, ***p<0.001 by one-way ANOVA with Tukey's multiple comparison test adjusted p value. FIG. 4E shows schema of the SH-SY5Y metastatic xenograft NB model in NSG mice inoculated intravenously via tail vein with 5×10⁵ of FFLuc-SH-SY5Y cells and treated 7 days later with 10×10⁶ of CAR-Ts intravenously. FIGS. 4F-4G provide representative tumor BLI images (FIG. 4F) and BLI kinetics (FIG. 4G) of FFLuc-SH-SY5Y tumor growth in the metastatic xenograft NB models shown in FIG. 4E (n=4 or 5 mice/group). FIG. 4H shows Kaplan-Meier survival curve of mice in FIGS. 4F-4G (n=4 or 5 mice/group); *p<0.05 by chi-square test. FIG. 4I shows the GD2 and B7-H3 expression level of tumor cells in the CD19.CAR and GD2.CAR-T cells treated mice were analyzed by flow cytometry at the time of the euthanasia.

FIGS. 5A-5H demonstrate GD2.CAR and B7-H3.CAR-T cells target neuroblastoma in vitro. FIG. 5A provides flow cytometry histogram showing the expression of GD2 in two human NB cell lines, CHLA-255 and LAN-1. FIG. 5B provides representative flow cytometry histograms showing the expression of CARs in human T cells transduced with retroviral vectors encoding the CARs of CD19.28ζ, GD2.28ζ, GD2.BBζ, B7-H3.28ζ, and B7-H3.BBζ. FIGS. 5C-5E provide representative flow cytometry plots (FIG. 5C) and quantification of residual CHLA-255 cells (FIG. 5D) and LAN-1 cells (FIG. 5E) labelled with GFP co-cultured with CAR-Ts at the T cell to tumor cell ratio of 1 to 5. On day 5, NB cells (GFP+) and CAR-Ts (CD3⁺) were enumerated by flow cytometry. Error bars denote SE, n=6; ns (not significant) by one-way ANOVA with Tukey's multiple comparison test adjusted p value. FIGS. 5F-5G show summary of IFN-γ (FIG. 5F) and IL-2 (FIG. 5G) released by CAR-Ts in the culture supernatant after 24 hours of co-culture with NB cells as measured by ELISA. Error bars denote SE, n=6; ns (not significant) by one-way ANOVA with Tukey's multiple comparison test adjusted p value. FIG. 5H shows representative CFSE dilution of CSFE-labeled CAR-Ts co-cultured with NB cells for 5 days at the T cell to tumor cell ratio of 1 to 1 (red histogram). CFSE-labeled CAR-Ts alone (grey histogram) were used as negative control.

FIGS. 6A-6D demonstrate that high doses of GD2.CAR-Ts and B7-H3.CAR-Ts with either CD28 or 4-1BB co-stimulation equally control tumor growth in vivo. FIG. 6A provides schema of the CHLA-255 metastatic xenograft NB model using NSG mice inoculated intravenously via tail vein with 2×10⁶ of FFluc-CHLA-255 cells and 14 days later received high doses of CAR-Ts (6×10⁶ cells/mouse) intravenously. FIGS. 6B-6C provide bioluminescence images (FIG. 6B) and bioluminescence kinetics (FIG. 6C) of FFluc-CHLA-255 tumor growth (n=5 mice/group) in the metastatic xenograft NB models shown in (FIG. 6A). FIG. 6D provides Kaplan-Meier survival curve of mice in (FIGS. 6B-6C) (n=5 mice/group).

FIGS. 7A-7D demonstrate GD2.28ζ.CAR-T has the strongest antitumor activity in vivo between GD2.CAR-T and B7-H3.CAR-T. FIG. 7A shows schema of the LAN-1 metastatic xenograft NB model using NSG mice inoculated intravenously via tail vein with FFLuc-LAN-1 cells and 21 days later received low doses of CAR-Ts intravenously targeting either GD2 or B7-H3. FIGS. 7B-7C provides bioluminescence images (FIG. 7B) and kinetics (FIG. 7C) of FFLuc-LAN-1 tumor growth (n=3 mice/group). FIG. 7D shows Kaplan-Meier survival curve of mice in FIGS. 7B-7C (n=3 mice/group).

FIGS. 8A-8E demonstrate tandem addition of 4-1BB and simply addition of B7-H3.BBζ does not improve antitumor activity of GD2.28ζ.CAR-T cells in vitro. FIG. 8A provides representative flow cytometry histograms showing the expression of CARs in human T cells transduced with retroviral vectors encoding the CARs of CD19.28ζ, GD2.28ζ, GD2.28.BBζ, or GD2.28ζ/B7-H3.28ζ. FIGS. 8B-8C provides representative flow cytometry plots (FIG. 8B) and quantification of residual CHLA-255 cells (FIG. 8C) labelled with GFP co-cultured with CAR-Ts at the T cell to tumor cell ratio of 1 to 5. Error bars denote SE, n=6; *p<0.05, **p<0.01, ***p<0.001, ns (not significant) by one-way ANOVA with Tukey's multiple comparison test adjusted p value. FIGS. 8D-8E show summary of IFN-γ (FIG. 8D) and IL-2 (FIG. 8E) released by CAR-Ts in the culture supernatant after 24 hours of co-culture with NB cells as measured by ELISA. Error bars denote SE, n=6; *p<0.05, **p<0.01, ***p <0.001, ns (not significant) by one-way ANOVA with Tukey's multiple comparison test adjusted p value.

FIGS. 9A-9E demonstrate one single shared CD3ζ chain is sufficient for transducing the activation signal in dual specific CAR-T cells. FIG. 9A shows schematic representation of retroviral vectors encoding B7-H3.BBζ, B7-H3.BBζ, GD2.28ζ, GD2.28ζ/B7-H3.BBζ, dNGFR.28ζ/B7-H3.BBζ, and 28ζ/B7-H3.BB. FIGS. 9B-9C provide representative flow cytometry plots (FIG. 9B) and percentage of residual GFP-labelled CHLA-255 cells (FIG. 9C) in coculture experiments in which CAR-T cells and tumor cells were plated at the T cell to tumor cell ratio of 1 to 5. Error bars denote SE, n=4; ns (not significant) by one-way ANOVA with Tukey's multiple comparison test adjusted p value. FIGS. 9D-9E show summary of IFN-γ (FIG. 9D) and IL-2 (FIG. 9E) released by CAR-Ts in the culture supernatant after 24 hours of co-culture with NB cells as measured by ELISA. Error bars denote SE, n=4; *p<0.05, ***p<0.001 by one-way ANOVA with Tukey's multiple comparison test adjusted p value.

FIGS. 10A-10B demonstrate memory phenotype after 13 days expansion in vitro. FIGS. 10A-10B shows phenotypic characterization of human T cells transduced with retroviral vectors encoding GD2.28ζ.CAR or GD2.28ζ/B7-H3.BB.CAR. Frequency of CD45RA+CCR7+, CD45RA-CCR7+, CCR7-CD28+CD27+, CCR7-CD28+CD27-, CCR7-CD28-CD27+, and CCR7-CD28-CD27—in CD4+(FIG. 10A) and CD8+(FIG. 10B) T cells on day 13 after transduction. Error bars denote SE, n=4.

FIGS. 11A-11D demonstrate phenotypic characteristics of CAR-Ts in vivo. FIGS. 11A-11B show phenotypic characterization of human T cells transduced with retroviral vectors encoding GD2.28ζ.CAR or GD2.28ζ.CAR/B7-H3.BB.CAR. Frequency of CD45RA+CCR7+, CD45RA-CCR7+, CCR7-CD28+CD27+, CCR7-CD28+CD27-, CCR7-CD28-CD27+, and CCR7-CD28-CD27—in CD4+(FIG. 11A) and CD8+(FIG. 11B) T cells on day 14 after CAR-T treatment. Error bars denote SE, n=5. FIGS. 11C-11D show Mean Fluorescence Intensity of PD-1 (FIG. 11C) and TIM-3 (FIG. 11D) in human T cells transduced with retroviral vectors encoding GD2.28ζ.CAR or GD2.28ζ.CAR/B7-H3.BB.CAR on day 14 after CAR-T treatment. Error bars denote SE, n=5; *p<0.05, ***p<0.001 by unpaired t test.

FIGS. 12A-12F demonstrate dual targeting, split signaling and one single CD3ζ endodomain promote TCR tonic signaling. FIGS. 12A-12C show scheme of CAR-T cell stimulation and sample preparation for RNAseq (FIG. 12A). Both GD2.28ζ.CAR-T cells and GD2.28ζ.CAR/B7-H3.BB.CAR-T cells were stimulated with 1 μg/mL 1A7 antibody and 1 μg/mL B7-H3-Fc protein coated plate, and CAR-T cells were collected for RNAseq at day 0, 1 and day 5 after stimulation. Gene set enrichment analysis (GSEA) (FIG. 12B) and qPCR validation (FIG. 12C) of TCR-related genes upregulated and down regulated in GD2.28ζ.CAR-T cells versus GD2.28ζ.CAR/B7-H3.BB.CAR-T cells in the absence of antigen stimulation. FIG. 12D shows basal phosphorylation of CAR-CD3ζ, Erk1/2, and Akt in GD2.28ζ.CAR-T cells and GD2.28ζ.CAR/B7-H3.BB.CAR-T cells in the absence of antigen stimulation. FIG. 12E shows time course of CAR-CD3ζ, Erk1/2, and Akt phosphorylation in GD2.28ζ.CAR-T cells and GD2.28ζ.CAR/B7-H3.BB.CAR-T cells after CAR cross-linking (1A7 Ab for GD2.CAR and B7-H3-Fc protein for B7-H3.CAR). FIG. 12F shows KEGG pathway analysis of top 100 loading genes in PC2 in FIG. 3I.

FIGS. 13A-13D demonstrate GD2.28ζ.CAR/B7-H3.BB.CAR-T cells have superior antitumor effects and preventing antigen escaping when targeting neuroblastoma tumor with heterogeneous GD2 expression in high tumor burden xenograft model. FIG. 13A shows schema of the high tumor burden SH-SY5Y metastatic xenograft NB model using NSG mice inoculated intravenously via tail vein with 1×10⁶ of FFLuc-SH-SY5Y cells and 7 days later received 10×10⁶ of CAR-T cells intravenously. FIGS. 13B-13C provide bioluminescence images (FIG. 13B) and bioluminescence kinetics (FIG. 13C) of FFLuc-SH-SY5Y tumor growth (n=5 mice/group) in the metastatic xenograft NB models shown in FIG. 13A. FIG. 13D shows Kaplan-Meier survival curve of mice in FIGS. 13B-13C (n=5 mice/group).

FIGS. 14-32 re-present certain data from FIGS. 1-13 and provide additional data.

FIGS. 14A-14H demonstrate single or dual antigen targeting and single or dual CD28 or 4-1BB costimulation do not eradicate the tumor in stress conditions. FIG. 14A provides a schema of the CHLA-255 metastatic xenograft NB model in NSG mice inoculated via tail injection with FFLuc-labelled CHLA-255 cells and treated 14 days later with low doses of CAR-T cells targeting either GD2 (GD2.28ζ and GD2.BBζ) or B7-H3 (B7-H3.28ζ and B7-H3.BBζ) or control CD19.28ζ. FIGS. 14B-14C provides representative tumor bioluminescence (BLI) images (FIG. 14B) and BLI kinetics (FIG. 14C) of FFLuc-CHLA-255 tumor growth in the metastatic xenograft NB model shown in FIG. 14A (n=5 mice/group). FIG. 14D shows Kaplan-Meier survival curve of mice in FIGS. 14B-14C (n=5 mice/group); Comparison of survival curves were determined by Log-rank test, **p=0.0027 for CD19.28ζ vs. all other groups, **p=0.0027 for GD2.28ζ vs. B7-H3.BBζ, GD2. BBζ vs. B7-H3.BBζ, and B7-H3.28λ vs. B7-H3.BBζ, **p=0.0018 for GD2.28ζ vs. GD2.BBζ, and GD2.28ζ vs. B7-H3.28ζ, **p=0.0018 for GD2. BBζ vs. B7-H3.28ζ. FIG. 14E provides a schema of the CHLA-255 metastatic xenograft NB model in NSG mice inoculated via tail vein injection with FFLuc-labelled CHLA-255 cells and treated 14 days later with low doses of GD2.28ζ, GD2.28.BBζ, GD2.28ζ/B7-H3.BBζ, and control CD19.28ζ CAR-T cells. FIGS. 14F-14G show representative tumor BLI (FIG. 14F) and BLI kinetics (FIG. 14G) of FFLuc-CHLA-255 tumor growth in the metastatic xenograft NB models shown in FIG. 14E (n=4 or 5 mice/group). FIG. 14H shows Kaplan-Meier survival curve of mice in (FIGS. 14F-14G) (n=4 or 5 mice/group); Comparison of survival curves were determined by Log-rank test, **p=0.0072 for CD19.28ζ vs. GD2.28ζ, **p=0.0027 for CD19.28ζ vs. GD2.28.BBζ and CD19.28ζ vs. GD2.28ζ/B7-H3.BBζ, *p=0.0350 for GD2.28ζ vs. GD2.28ζ/B7-H3.BBζ, *p=0.0157 for GD2.28.BBζ vs. GD2.28ζ/B7-H3.BBζ.

FIGS. 15A-15E demonstrate one single shared CD3ζ chain is sufficient for transducing the activation signal in dual specific CAR-T cells. FIG. 15A provides a schematic representation of the retroviral vectors encoding B7-H3.BBζ, B7-H3.BBζ, GD2.28ζ, 28ζ, dNGFR.28ζ, GD2.28ζ/B7-H3.BBζ, GD2.28ζ/dNGFR.BBζ, dNGFR.28ζ/B7-H3.BB and 28ζ/B7-H3.BB. scFv.14.g2a, single-chain variable fragment of the anti-GD2 monoclonal antibody 14.g2a; scFv.276.96, single-chain variable fragment of the anti-B7-H3 monoclonal antibody 376.96; CD8α, the stalk and transmembrane region of human CD8α; CD28, intracellular domain of human CD28; 4-1BB, intracellular domain of human 4-1BB; CD3ζ, intracellular domain of human CD3ζ chain; dNGFR, extracellular domain of human nerve growth factor receptor. FIG. 15B shows representative flow cytometry plots showing residual GFP-labelled CHLA-255 cells in co-culture experiments in which CAR-T cells and tumor cells were plated at the T cell to tumor cell ratio of 1 to 5, and tumor cells (GFP⁺) and T cells (CD3⁺) were numerated by flow cytometry at 5 days after co-culture. Representative of 4 independent experiments. FIGS. 15C-15E provides summary of residual tumor cells (FIG. 15C), IFN-γ (FIG. 15D) and IL-2 (FIG. 15E) released by CAR-T cells in the co-culture experiments described in FIG. 15B. NT, Non-transduced T cell; Data are shown as individual values and the mean+SD, n=4 independent co-culture with CAR-T cells generated from 4 different donors for NT and dNGFR.28ζ/B7-H3.BB groups, n=6 independent co-culture with CAR-T cells generated from 6 different donors for other groups; *p=0.0119 in FIG. 15E, **p=0.0027 for GD2.28ζ/B7-H3.BB vs. GD2.28ζ/dNGFR.BB and **p=0.0051 for GD2.28ζ/B7-H3.BB vs. 28ζ/B7-H3.BB in FIG. 15D, ***p=0.0002 for B7-H3.BBζ vs. GD2.28ζ/B7-H3.BB and ***p=0.0005 for GD2.28ζ vs. GD2.28ζ/B7-H3.BB in FIG. 15D, ***p=0.0001 in FIG. 15E, ****p<0.0001 in FIGS. 15C-15E by one-way ANOVA with Tukey's multiple comparison test adjusted p value.

FIGS. 16A-16G demonstrate CD3ζ sharing in the dual CAR relies on CD8α-mediated dimerization. FIGS. 16A-16B show T cells co-expressing B7-H3.BB and dNGFR.28ζ or 28ζ were stimulated with the B7-H3-Fc protein followed by incubation with an anti-Fc secondary Ab for 20 minutes at 37° C. Cells were then lysed in Laemmli buffer in non-reducing (without β-mercaptoethanol) (FIG. 16A) or reducing (with β-mercaptoethanol) (FIG. 16B) conditions for 10 minutes at 100° C., and separated on non-reducing gel or reducing gels. Membranes were stained with the anti-CD3ζ antibody. Data are representative of two independent experiments in FIGS. 16A-16B. FIG. 16C provides a schematic representation of the retroviral vectors encoding dNGFR.28ζ/B7-H3.BBζ CD8m) and 28ζ/B7-H3.BBζ CD8m). CD8m, the stalk and transmembrane region of human CD8α that carrying the C164S and C181S mutations. FIGS. 16D-16F provides a summary of residual tumor cells (FIG. 16D), IFN-γ (FIG. 16E) and IL-2 (FIG. 16F) in the co-culture experiments of CAR-T cells with CHLA-255 at T cell to tumor cell ratio of 1 to 5. Data are shown as individual values and the mean+SD, n=4 independent co-culture with CAR-T cells generated from 4 different donors; ****p<0.0001 by one-way ANOVA with Tukey's multiple comparison test adjusted p value. FIG. 1G shows representative confocal microscopy imaging showing CARs clustering in T cells expressing GFP-tagged GD2.28ζ (green) and B7-H3.BB (red) with and without CAR engagement using either the anti-14g2a idiotype antibody (1A7) or the B7-H3-Fc protein. Blue staining indicates the DAPI. Shown are representative cells. Data are representative of three independent validations. Shown in white are the scale bars that correspond to 5 μm.

FIGS. 17A-17Q demonstrate dual targeting with split costimulation and shared single CD3ζ promotes sustained antitumor activity. FIG. 17A provides representative flow cytometry plots showing the expression of CARs in CAR-T cells. FIG. 17B provides a summary of CARs transduction efficiency (n=9 independent experiments); data are shown as individual values and the mean+SD. FIG. 17C provides a schema of the multi-rounds co-culture experiment. Tumor cells were seeded in 24-well plates one day prior to the addition of T cells. At day 0, CAR-T cells were added at T cell to tumor cell ratio of 1 to 5. At days 4, 6, and 8, all T cells were collected and transferred into a new well in which 5×10⁵ NB cells were seeded one day before. T cells and NB cells were quantified by flow cytometry after each cycle. Supernatants were also collected for cytokine measurements 24 hours after adding T cells for each cycle. FIGS. 17D-17K show multi-rounds co-culture experiments with CHLA-255 (FIGS. 17D-17G) and LAN-1 (FIGS. 17H-17K) cells. Quantification of residual tumor cells (FIG. 17D, FIG. 17H) and enumeration of T cells (FIG. 17E, FIG. 17I), and summary of IFN-γ (FIG. 17F, FIG. 17J) and IL-2 (FIG. 17G, FIG. 17K) released by CAR-T cells in the multi-rounds co-culture experiments. Data are shown as individual values and the mean+SD, n=12 independent co-culture with CAR-T cells generated from 12 different donors; *p <0.05, **p<0.01, ***p<0.001, ****p<0.0001, by one-way ANOVA with Tukey's multiple comparison test adjusted p value, the full list of p values can be found in the source data. FIG. 17L provides a schema of the CHLA-255 metastatic xenograft NB model. FIGS. 17M-17N show representative tumor BLI images (FIG. 17M) and BLI kinetics (FIG. 17N) of FFLuc-CHLA-255 tumor growth in the tumor models shown in (FIG. 17L) (n=5 mice/group). FIG. 17O shows Kaplan-Meier survival curve of mice in (FIGS. 17M-17N) (n=5 mice/group); **p<0.01 by Log-rank test. FIGS. 17P-17Q provide a summary of circulating CAR-T cells (CD45+CD3+) in mice 14 days (FIG. 17P) and 28 days (FIG. 17Q) after CAR-T cell treatment (n=3 mice in GD2.28ζ group in FIG. 17Q, n=5 mice/group in other groups), Data are shown as individual values and the mean+SD, **p=0.0038 for CD19.28ζ vs. GD2.28ζ/B7-H3.BB and **p=0.0044 for GD2.28ζ vs. GD2.28ζ/B7-H3.BB in FIG. 17P, adjusted p value by one-way ANOVA with Tukey test for multiple comparison.

FIGS. 18A-18I demonstrate MSLN and CSPG4 dual targeting CAR-T cells with split co-stimulation and shared CD3ζ show sustained T cell activation and proliferation in vitro and in vivo. FIG. 18A provides a schematic representation of retroviral vectors encoding CSPG4.BBζ, CSPG4.BBζ, MSLN.28ζ and MSLN.28ζ/CSPG4.BB CARs. scFv.763.74, single-chain variable fragment of the anti-CSPG4 monoclonal antibody 763.74; Amatuximab, single-chain variable fragment of the anti-MSLN monoclonal antibody amatuximab; CD8α, stalk and transmembrane region of human CD8α; CD28, intracellular domain of human CD28; 4-1BB, intracellular domain of human 4-1BB; CD3ζ, intracellular domain of human CD3ζ chain; F, Flag-tag. FIG. 18B provides flow cytometry histograms showing the expression of MSLN and CSPG4 in the human mesothelioma cell line H2052. Representative of three independent experiments. FIGS. 18C-18D provide a summary of the number of residual H2052 cells (FIG. 18C) and T cells (FIG. 18D) in the multi-round co-culture experiments with H2052 tumor cells. Data are shown as individual values and the mean+SD, n=3 independent experiments with CAR-T cells generated from 3 different donors for the CSPG4.BBζ group, n=4 independent experiments with CAR-T cells generated from 4 different donors for the other groups; *p<0.05, **p<0.01, ***p<0.001, ****p<0.0001 by one-way ANOVA with Tukey's multiple comparison test adjusted p value, the full list of p values can be found in the source data. FIG. 18E shows a schema of the H2052 intraperitoneal xenograft model in NSG mice. Eight to 10 week old female NSG mice were inoculated with 3×10⁶ FFLuc-labelled H2052 cells by intraperitoneal injection, and treated 12 days later with 2×10⁶ CD19.28ζ, CSPG4.BBζ, MSLN.28ζ or MSLN.28ζ/CSPG4.BB CAR-T cells by intraperitoneal injection. FIGS. 18F-18G show representative tumor BLI images (FIG. 18F) and BLI kinetics (FIG. 18G) of FFLuc-H2052 tumor growth in the mesothelioma xenograft model shown in FIG. 18A. FIG. 18H shows Kaplan-Meier survival curve of mice in FIGS. 18F-18G, n=6 mice for the CD19.28ζ group, n=8 mice for the other groups; ***p=0.0003, ****p<0.0001 by Log-rank test. FIG. 18I shows detection of circulating CAR-T cells (CD45+CD3+) in mice 19 days after CAR-T cell treatment by flow cytometry; data are shown as individual values and the mean+SD, n=6 mice for CD19.28ζ group, n=8 mice for the other groups, *p<0.05 (0.0103 for CSPG4.BBζ vs. Co, 0.0159 for MSLN.28ζ vs. Co), **p=0.0084 by one-way ANOVA with Tukey's multiple comparison test adjusted p value.

FIGS. 19A-19I demonstrate dual targeting with split co-stimulation and shared D3(promote TCR tonic signaling. FIG. 19A shows a schema of CAR-T cell stimulation and sample preparation for RNAseq. Both GD2.28ζ and GD2.28ζ/B7-H3.BB CAR-T cells were stimulated with 1 μg/mL 1A7 Ab and 1 μg/mL B7-H3-Fc protein coated plate for 24 hours, and then transferred to a new plate without any pre-coating and cultured for 4 more days. CAR-T cells were collected for RNAseq at days 0, 1 and 5. FIG. 19B provides a RNAseq analysis of non-stimulated GD2.28ζ and GD2.28ζ/B7-H3.BB CAR-T cells. FIGS. 19C-19F show gene set enrichment analysis (GSEA) of glycolytic (FIG. 19C), IFN-γ signaling pathways (FIG. 19D), TCR upregulated (FIG. 19E) and downregulated genes (FIG. 19F) in non-stimulated CAR-T cells expressing GD2.28ζ or GD2.28ζ/B7-H3.BB. FIG. 19G shows qPCR validation of TCR-related genes upregulated and down regulated in GD2.28ζ vs. GD2.28ζ/B7-H3.BB CAR-T cells in the absence of antigen stimulation; n=4 independent donors, and data are shown as individual values and the mean+SD in C, *p<0.05, **p<0.01, two-tailed p value determined by unpaired t test. FIG. 19H shows basal phosphorylation of CAR-CD3ζ, Erk1/2, and Akt in GD2.28ζ and GD2.28ζ/B7-H3.BB CAR-T cells in the absence of antigen stimulation. Data are from one experiment, representative of three independent experiments. FIG. 19I shows time course of CAR-CD3ζ, Erk1/2, and Akt phosphorylation in GD2.28ζ and GD2.28ζ/B7-H3.BB CAR-T cells after CAR cross-linking (1A7 Ab for GD2.CAR and B7-H3-Fc protein for B7-H3.CAR). Data are from one experiment, representative of three independent experiments.

FIGS. 20A-20I demonstrate dual targeting with split co-stimulation and shared CD3ζ promote CAR-T cell proliferation, and glycolytic and oxidative metabolism. FIGS. 20A-20B show RNAseq analysis of GD2.28ζ and GD2.28ζ/B7-H3.BB CAR-T cells at day 1 (FIG. 2OA) and day 5 (FIG. 2OB) after CAR stimulation. FIGS. 20C-20D show GSEA of the cell cycle (FIG. 2OC) and TCR (FIG. 2OD) signaling five days after CAR stimulation. FIG. 2OE shows principal component analysis of transcriptome data from GD2.28ζ and GD2.28ζ/B7-H3.BB CAR-T cells at days 0, 1 and 5. FIGS. 20F-20G shows proliferation of GD2.28ζ and GD2.28ζ/B7-H3.BB CAR-T cells after CAR stimulation. FIG. 2OF shows CAR-T cells were stained with CFSE and then stimulated via 1A7 Ab and B7-H3-Fc protein on day 0, the CFSE dilutions were examined by flow cytometry on days 3 and 6 after stimulation. Representative of 4 independent experiments. FIG. 2OG shows T cell numbers were counted by flow cytometry with counting beads in a separate experiment without CFSE staining (n=5 independent experiments with CAR-T cells generated from 5 different donors), Error bars denote SD, *p<0.0001 determined by multiple unpaired t test with Holm-Sidak correction for multiple comparison. FIGS. 20H-20I provide metabolic profile showing glucose (FIG. 2OH) and O₂ consumption (FIG. 20I) of GD2.28ζ and GD2.28ζ/B7-H3BB CAR-T cells before CAR activation (resting), and days 1 and 5 after CAR activation. Extracellular acidification rate (ECAR) and O₂ consumption rate (OCR) were assayed at different time points in a Seahorse XF24 analyzer, n=3 independent experiments, Error bars denote SD, *p<0.05, **p<0.01, ***p<0.001,****p<0.0001, two-way ANOVA with Sidak correction for multiple comparison, the full list of p values can be found in the source data. The long and short arrows indicate the time point of adding Rot/AA and 2-DG respectively (FIG. 20H); the black, green and purple arrows indicate the time point of adding oligomycin, FCCP, Rot/AA respectively (FIG. 2OI).

FIGS. 21A-21J demonstrate dual targeting, split signaling and one single CD3ζ endodomain prevent tumor escape due to antigen loss. FIG. 21A shows flow cytometry histogram showing the expression of GD2 and B7-H3 in the NB cell line SH-SY5Y. Representative of 3 independent experiments. FIG. 21B shows quantification of the GD2 density on the cell membrane of CHLA-255, LAN-1 and SH-SY5Y cells as measured by flow cytometry. The numbers within bars indicate the calculated number of GD2 molecules on the cell membrane of each cell line. Representative of 2 independent experiments. FIG. 21C shows quantification of residual NB cells labelled with GFP and co-cultured with CD19.28ζ, GD2.28ζ and GD2.28ζ/B7-H3.BB CAR-T cells at the T cell to tumor cell ratio of 1 to 5. On day 5, NB cells (GFP+) and CAR-T cells (CD3⁺) were enumerated by flow cytometry. Data are shown as individual values and the mean+SD, n=10 independent co-culture with CAR-T cells generated from 10 different donors; **p=0.0011, ****p<0.0001 by one-way ANOVA with Tukey's multiple comparison test adjusted p value. FIGS. 21D-21E provides a summary of IFN-γ (FIG. 21D) and IL-2 (FIG. 21E) released by CAR-T cells in the culture supernatant after 24 hours of co-culture with NB cells as measured by ELISA. Data are shown as individual values and the mean+SD, n=10 independent co-culture with CAR-T cells generated from 10 different donors; ***p=0.0001 for CD19.28ζ vs. GD2.28ζ/B7-H3.BB and ***p=0.0002 for GD2.28ζ vs. GD2.28ζ/B7-H3.BB in FIG. 21D, ****p<0.0001 in FIG. 21E by one-way ANOVA with Tukey's multiple comparison test adjusted p value. FIG. 21F shows a schema of the SH-SY5Y metastatic xenograft NB model in NSG mice inoculated intravenously via tail vein with 5×10⁵ of FFLuc-SH-SY5Y cells and treated 7 days later with 1×10 CD19.28ζ, GD2.28ζ and GD2.28ζ/B7-H3.BB CAR-T cells intravenously. FIGS. 21G-21H shows representative tumor BLI images (FIG. 21G) and BLI kinetics (FIG. 21H) of FFLuc-SH-SY5Y tumor growth in the metastatic xenograft NB models shown in FIG. 21F (n=4 or 5 mice/group). FIG. 21I shows Kaplan-Meier survival curve of mice in FIGS. 21G-21H (n=4 or 5 mice/group); **p=0.0027 by Log-rank test. FIG. 21J shows GD2 and B7-H3 expression levels in tumor cells collected from mice treated with CD19.28ζ or GD2.28ζ CAR-T cells were analyzed by flow cytometry at the time of the euthanasia. Representative of 3 independent experiments.

FIGS. 22A-22H demonstrate GD2-specific CAR-T cells and B7-H3-specific CAR-T cells target neuroblastoma in vitro. FIG. 22A provides a flow cytometry histogram showing the expression of GD2 and B7-H3 in two human NB cell lines, CHLA-255 and LAN-1. Representative of three independent experiments. FIG. 22B provides representative flow cytometry histograms showing the expression of CARs in human T cells transduced with retroviral vectors encoding CD19.28ζ, GD2.28ζ, GD2.BBζ, B7-H3.28ζ, and B7-H3.BBζ CARs. FIGS. 22C-22E provide representative flow cytometry plots (FIG. 22C) and quantification of residual CHLA-255 (FIG. 22D) and LAN-1 (FIG. 22E) cells labelled with GFP and co-cultured with CAR-T cells at the T cell to tumor cell ratio of 1 to 5. On day 5, NB cells (GFP+) and CAR-T cells (CD3⁺) were enumerated by flow cytometry. Data are shown as individual values and the mean+SD, n=6 independent co-cultures using CAR-T cells generated from 6 different donors. FIGS. 22F-22G provide a summary of IFN-γ (FIG. 22F) and IL-2 (FIG. 22G) released by CAR-T cells in the culture supernatant after 24 h of co-culture with NB cells as measured by ELISA. Data are shown as individual values and the mean+SD, n=6 independent co-cultures using CAR-T cells generated from 6 different donors. FIG. 22H shows representative CFSE dilution of CSFE-labeled CAR-T cells co-cultured with NB cells for 5 days at the T cell to tumor cell ratio of 1 to 1 (red histogram). CFSE-labeled CAR-T cell alone (grey histogram) was used as negative control. Representative of three independent experiments.

FIGS. 23A-23H demonstrate the antitumor activity of GD2-specific CAR-T cells and B7-H3-specific CAR-T cells with either CD28 or 4-1BB costimulation in vivo. FIG. 23A provides a schema of the CHLA-255 metastatic xenograft NB model using NSG mice inoculated via tail vein injection with 2×10⁶ of FFluc-CHLA-255 cells and 14 days later received high doses of CAR-T cells (6×10⁶ cells/mouse) intravenously. FIGS. 23B-23C provides representative tumor bioluminescence (BLI) images (FIG. 23B) and tumor BLI kinetics (FIG. 23C) of FFluc-CHLA-255 tumor growth (n=3 mice for the CD19.28ζ group, n=5 mice for the other four groups) in the metastatic xenograft NB models shown in FIG. 23A. FIG. 23D shows Kaplan-Meier survival curve of mice in FIGS. 23B-23C, n=3 mice for CD19.28ζ group, n=5 mice for other 4 groups, comparisons of survival curves were determined by Log-rank test, **p=0.0042 for CD19.28ζ versus other 4 groups. FIG. 23E shows a schema of the LAN-1 metastatic xenograft NB model using NSG mice inoculated via tail vein injection with FFLuc-LAN-1 cells and treated 21 days later with low doses CD19.28ζ, GD2.28ζ, GD2.BBζ, B7-H3.28ζ or B7-H3.BBζ CAR-T cells intravenously. FIGS. 23F-23G provide representative tumor BLI images (FIG. 23F) and tumor BLI kinetics (FIG. 23G) of FFLuc-LAN-1 tumor growth (n=3 mice/group). FIG. 23H shows Kaplan-Meier survival curve of mice in FIGS. 23F-23G, n=3 mice/group, comparisons of survival curves were determined by Logrank test, *p=0.0253 for CD19.28ζ versus GD2.28ζ, GD2.BBζ and B7-H3.BBζ groups, *p=0.0295 for GD2.28ζ versus GD2.BBζ, *p=0.0246 for GD2.28ζ versus B7-H3.BBζ.

FIGS. 24A-24E demonstrate addition of 4-1BB in tandem to the GD2.28ζ CAR and co-expression of both GD2.28ζ and B7-H3.BBQ CARs do not improve antitumor activity in vitro. FIG. 24A provides representative flow cytometry plots showing the CAR expression in human T cells transduced with retroviral vectors encoding CD19.28ζ, GD2.28ζ, GD2.28.BBζ, or GD2.28ζ/B7-H3.28ζ CARs. Representative of six independent experiments. FIGS. 24B-24C provide representative flow cytometry plots (FIG. 24B) and quantification of residual CHLA-255 cells (FIG. 24C) labelled with GFP co-cultured with CAR-T cells at the T cell to tumor cell ratio of 1 to 5. Data are shown as individual values and the mean+SD, n=6 or 8 independent co-cultures using CAR-T cells generated from 6 or 8 different donors. FIGS. 24D-24E provide a summary of IFN-γ (FIG. 24D) and IL-2 (FIG. 24E) released by CAR-T cells in the culture supernatant after 24 h of co-culture with NB cells as measured by ELISA. Data are shown as individual values and the mean+SD, n=6 or 8 independent co-cultures using CAR-T cells generated from 6 or 8 different donors; **p=0.0011, ****p<0.0001 by one-way ANOVA with Tukey's multiple comparison test adjusted p value.

FIGS. 25A-25G demonstrate cytotoxic activity of the double CAR-T cells with shared CD3ζ is antigen dependent. FIG. 25A provides flow cytometry plots showing the expression of B7-H3 and GD2 in Raji cells wild type and B7-H3 expression in Raji cells transduced with a retroviral vector encoding B7-H3 (Raji-B7-H3). Representative of three independent experiments. FIGS. 25B-25D show CAR-T cells (B7-H3.BBζ, B7-H3.BBζ, GD2.28ζ, GD2.28ζ/B7-H3.BBζ, dNGFR.28ζ/B7-H3.BB and 28ζ/B7-H3.BB) were co-cultured with Raji-B7-H3 cell at 1 to 1 ratio, and 5 days later tumor cells (CD19+) and T cells (CD3⁺) were collected and enumerated by flow cytometry (FIG. 25B). Supernatants of the co-cultures were collected 24 h later, and IFN-γ (FIG. 25C) and IL-2 (FIG. 25D) released by CAR-T cells were measured by ELISA. Data are shown as individual values and the mean+SD, n=3 independent co-cultures using CAR-T cells generated from 3 different donors for dNGFR.28ζ/B7-H3.BB group, and n=5 independent co-cultures using CAR-T cells generated from 5 different donors for all the other groups; *p<0.05 (0.0228 in FIG. 25C, 0.0141 in FIG. 25D), **p<0.01 (0.0025 in FIG. 25C, 0.0015 in FIG. 25D), ***p =0.0005, ****p<0.0001 by one-way ANOVA with Tukey's multiple comparison test adjusted p value. FIGS. 25E-25G show CAR-T cells (CD19.28ζ, GD2.28ζ/B7-H3.BBζ, dNGFR.28ζ/B7-H3.BB and 28ζ/B7-H3.BB) were co-cultured with Raji cell wild type at 1 to 1 ratio, and 5 days later tumor cells (CD19+) and T cells (CD3⁺) were collected and enumerated by flow cytometry (FIG. 25E). Supernatants of the co-cultures were collected 24 h later, and IFN-γ (FIG. 25F) and IL-2 (FIG. 25G) released by CAR-T cells were measured by ELISA. Data are shown as individual values and the mean+SD, n=4 independent co-cultures using CAR-T cells generated from 4 different donors for each group; ****p<0.0001 by one-way ANOVA with Tukey's multiple comparison test adjusted p value.

FIG. 26 shows CAR clustering and aggregation in CAR-T cells after CAR engagement. Representative confocal microscopy imaging showing CAR molecule clustering in T cells expressing GFP-tagged GD2.28ζ (green) and B7-H3.BB (red) with and without engagement of the CARs using either the anti-14g2a idiotype antibody (1A7) or the B7-H3.Fc protein. Blue staining indicates the DAPI. Shown are representative cells of a single field (Magnification 63×). Data are representative of three independent validations. Shown in white are the scale bars that correspond to 20 m.

FIGS. 27A-27F demonstrate phenotypic analysis of CAR-T cells in vitro and in vivo. FIGS. 27A-27B show frequency of CD45RA+CCR7+, CD45RA-CCR7+, CCR7-CD28+CD27+, CCR7-CD28+CD27-, CCR7-CD28-CD27+, and CCR7-CD28-CD27—in CD4+(FIG. 27A) and CD8+(FIG. 27B) T cells on day 13 after retroviral vector transduction and expansion in vitro. Data are shown as individual values and the mean+SD, n=4 independent experiments using CAR-T cells generated from 4 different donors; *p=0.0299, two-tailed p value determined by unpaired t test. FIGS. 27C-27F show tumor-baring mice infused with CAR-T cells were bled at day 14 and CAR-T cells in the peripheral blood were analyzed by flow cytometry. FIGS. 27C-27D show frequency of CD45RA+CCR7+, CD45RA-CCR7+, CCR7-CD28+CD27+, CCR7-CD28+CD27-, CCR7-CD28-CD27+, and CCR7-CD28-CD27—in CD4+(FIG. 27C) and CD8+(FIG. 27D) T cells. Data are shown as individual values and the mean +SD, n=5 samples from 5 mice, *p<0.05, two-tailed p value determined by unpaired t test. FIGS. 27E-27F show Mean Fluorescence Intensity (MFI) of PD-1 (FIG. 27E) and TIM-3 (FIG. 27F) in T cells. Data are shown as individual values and the mean+SD, n=5 samples from 5 mice; *p=0.0109, ***p=0.0008, two-tailed p value determined by unpaired t test.

FIGS. 28A-280 demonstrate inverting the orientation of the B7-H3-specific CAR and GD2-specific CAR does not alter the beneficial effects of dual targeting CAR-T cells with split costimulation and shared CD3ζ in vitro. FIG. 28A provides a schematic representation of retroviral vectors encoding B7-H3.28ζ, GD2.BB and B7-H3.28ζ/GD2.BB CARs. FIG. 28B provides representative flow cytometry plots of 5 independent experiments showing the expression of CARs. FIG. 28C provides a summary of the transduction efficiency of the CARs. Data are shown as individual values and the mean+SD, n=5 or 7 independent experiments using CAR-T cells generated from 5 or 7 different donors; ***p=0.002, ****p<0.0001 by one-way ANOVA with Tukey's multiple comparison test adjusted p value. FIGS. 28D-28F show CAR-T cells were co-cultured with CHLA-255-GFP at T cell to tumor cell ratio of 1 to 5. IFN-γ (FIG. 28E) and IL-2 (FIG. 28F) released by CAR-T cells were measured by ELISA. On day 5, tumor cells (GFP+) and CAR-T cells (CD3⁺) number were measured by flow cytometry (FIG. 12D). Data are shown as individual values and the mean+SD, n=3 independent co-cultures using CAR-T cells generated from 3 different donors; *p<0.05, **p<0.01, ***p<0.001, ****p<0.0001 by oneway ANOVA with Tukey's multiple comparison test adjusted p value, the full list of p values can be found in the source data. FIG. 28G shows a schema of the repetitive multi-round co-culture experiments. Tumor cells were seeded in 24-well plates one day prior to the addition of T cells. At day 0, CAR-T cells were added at T cell to tumor cell ratio of 1 to 5. At day 4, 7, and 10, all T cells were collected and transferred into a new well in which 5×10⁵ NB cells were seeded one day before. T cells and tumor cells, and cytokine were quantified at each cycle. FIGS. 28H-280 show multi-round co-culture with NB cell lines CHLA-255 (FIGS. 28H-28K) and LAN-1 (FIGS. 28L-280) cells as described in FIG. 28G. Summary of percentage of residual CHLA-255 (FIG. 28H) and LAN-1 (FIG. 28L) cells and number of T cells (FIG. 28I, FIG. 28M) at the end of each round of co-culture. Summary of IFN-γ (FIG. 28J, FIG. 28N) and IL-2 (FIG. 28K, FIG. 28O) released by CAR-T cells in the culture supernatant after 24 h of co-culture with CHLA-255 (FIGS. 28J-28K) and LAN-1 (FIGS. 28N-280) cells. Data are shown as individual values and the mean+SD, n=6 independent co-cultures using CAR-T cells generated from 6 different donors; *p <0.05, **p<0.01, ***p<0.001, ****p<0.0001 by one-way ANOVA with Tukey's multiple comparison test adjusted p value, the full list of p values can be found in the source data.

FIGS. 29A-29E demonstrate dual targeting with split co-stimulation and shared CD3ζ provide superior antitumor activity and better T cell persistence in NB model when mice are treated with inverted B7-H3-specific CAR and GD2-specific CAR. FIG. 29A shows a schema of the CHLA-255 metastatic xenograft NB model in NSG mice. Eight week old female NSG mice were inoculated with 2×10⁶ FFLuc-labelled CHLA-255 cells via tail vein injection, and 14 days later mice were treated with 2×10⁶ CD19.28ζ, B7-H3.28ζ or B7-H3.28ζ/GD2.BB CAR-T cells via tail vein injection. FIGS. 29B-29C show representative tumor bioluminescence (BLI) images (FIG. 29B) and tumor BLI kinetics (FIG. 29C) of FFLuc-CHLA-255 tumor growth in the metastatic xenograft NB model shown in FIG. 29A (n=3 mice for the CD19.28ζ group, n=5 mice for the other two groups). FIG. 29D shows Kaplan-Meier survival curve of mice in FIGS. 29B-29C (n=3 for the CD19.28ζ group, n=5 for the other two groups); **p=0.0016 (B7-H3.28λ vs. B7-H3.28ζ/GD2.BB) by Log-rank test. FIG. 29E shows detection of circulating CAR-T cells (CD45+CD3+) in mice 14 days after CAR-T cell treatment by flow cytometry. Data are shown as individual values and the mean+SD, (n=3 samples from 3 mice for the CD19.28ζ group, n=5 samples from 5 mice for the other two groups); *p=0.0144, **p=0.0042 by one-way ANOVA with Tukey's multiple comparison test adjusted p value.

FIGS. 30A-30H demonstrate MSLN and CSPG4 dual targeting CAR-T cells with split co-stimulation and shared CD3ζ show sustained T cell activation and proliferation in vitro. FIG. 30A provide representative flow cytometry plots showing the expression of CARs. FIG. 30B show summary of the transduction efficiency of the CARs (n=7 or 9 independent experiments using CAR-T cells generated from 7 or 9 different donors). Data are shown as individual values and the mean+SD. FIGS. 30C-30E show CAR-T cells co-cultured with GFP labeled H2052 cell at T cell to tumor cell ratio of 1 to 5. IFN-γ (FIG. 30D) and IL-2 (FIG. 30E) released by CAR-T cells. On day 5, tumor cells (GFP+) and CAR-T cells (CD3⁺) were measured by flow cytometry (FIG. 30C). Data are shown as individual values and the mean+SD, n=3 independent co-cultures using CAR-T cells generated from 3 different donors for the CSPG4.BBζ group, n=5 independent co-cultures using CAR-T cells generated from 5 different donors for the other groups; *p<0.05, **p<0.01, ***p<0.001, ****p <0.0001 by one-way ANOVA with Tukey's multiple comparison test adjusted p value, the full list of p values can be found in the source data. FIG. 30F shows a schema of the multi-round co-culture experiments of CAR-T cells and H2052. Tumor cells were seeded one day prior to the addition of T cells. At day 0, CAR-T cells were added at T cell to tumor cell ratio of 1 to 5. At the end of each round of co-culture, which are at days 5, 9, 13 and 17, one third of T cells were collected and transferred into a new well with 2.5×10⁵ H2052 cells that were seeded one day before. T cells and tumor cells and cytokine released by CAR-T cells were quantified at each round of co-culture. FIGS. 30G-30H show summary of IFN-γ (FIG. 30G) and IL-2 (FIG. 30H) released by CAR-T cells in the multi-round co-culture with H2052 as described in FIG. 30F. Data are shown as individual values and the mean+SD, n=3 independent co-cultures using CAR-T cells generated from 3 different donors for the CSPG4.BBζ 4 group, n=4 independent co-cultures using CAR-T cells generated from 4 different donors for the other groups; *p<0.05, **p<0.01, ***p<0.001, ****p<0.0001 by one-way ANOVA with Tukey's multiple comparison test adjusted p value, the full list of p values can be found in the source data.

FIGS. 31A-31D demonstrate dual specific GD2 and B7-H3 CAR-T cells with split costimulation and shared CD3z have superior antitumor activity and prevent antigen escape in high tumor burden xenograft model with neuroblastoma cells showing heterogeneous GD2 expression. FIG. 3IA shows a schema of the high tumor burden SH-SY5Y metastatic xenograft NB model using NSG mice inoculated via tail vein injection with FFLuc-SH-SY5Y cells (1×10⁶ cell/mouse) and treated 7 days later with CD19.28; GD2.28ζ or GD2.28ζ/B7-H3BB CAR-T cells (1×10⁷ cells/mouse) intravenously. FIGS. 31B-31C provide representative tumor bioluminescence (BLI) images (FIG. 3IB), and tumor BLI kinetics (FIG. 3IC) of FFLuc-SH-SY5Y tumor growth (n=5 mice/group) in the metastatic xenograft NB models shown in FIG. 3IA. FIG. 3ID shows Kaplan-Meier survival curve of mice in FIGS. 31B-31C, n=5 mice/group, comparisons of survival curves were determined by Log-rank test, **p=0.0023 for CD19.28ζ vs. GD2.28ζ, **p=0.0027 for GD2.28ζ vs. GD2.28ζ/B7-H3.BB.

FIG. 32 shows KEGG pathway analysis of top 100 loading genes in PC2 in FIG. 2OE. The nominal p values and FDR q values were calculated using GSEA software (Broad Institute).

### DETAILED DESCRIPTION OF THE INVENTION

Preventing tumor escape due to heterogeneity in antigen expression and providing optimal T cell co-stimulation remain critical aspects to achieving a clinical response with CAR T cells in solid tumors. Described herein are CAR-T cells that simultaneously target two antigens and provide optimal co-stimulation and T cell metabolic fitness by activating independently CD28 and 4-1BB pathways and tuning CD3ζ-chain-mediated signaling. The modified T cells expressing a dual CAR provide robust and sustained antitumor activity in in vivo stress conditions and prevent tumor escape due to heterogeneous antigen expression by tumor cells.

Modified T Cells

Aspects of the disclosure relate to modified T cells. In some embodiments, the modified T cells are chimeric antigen receptor (CAR)-engineered T cells. CAR T cells are produced by obtaining T cells, such as from a subject in need thereof or from a donor subject, and manipulating the cells such that they include chimeric antigen receptors (CARs). The CARs provide the ability to target specific proteins on cancer cells and typically include an antigen recognition domain, an extracellular hinge region, a transmembrane domain, and an intracellular T cell signaling domain. CAR T cells may be classified as first generation, second generation, third generation, or fourth generation. First generation CARs were engineered with only the CD3ζ domain. Second generation CARs were engineered with the CD3ζ domain and a co-stimulatory signaling domain (e.g., CD28 or 4-1BB). Third generation CARs are engineered to include the CD3ζ domain in addition to two co-stimulatory signaling domains (e.g., both CD28 and CD137). Finally, fourth generation CARs, also referred to as T-cells redirected for universal cytokine-mediated killing (TRUCKs) are engineered to include the CD3ζ domain, two co-stimulatory signaling domains (e.g., both CD28 and CD137), and some additional genetic modification, such as the addition of transgenes for cytokine secretion or additional co-stimulatory signaling domains. Described herein are modified T cells comprising CARs providing dual specificity and dual co-stimulation.

In some embodiments, the T cell is a human T cell or a non-human T cell. In some embodiments mammalian cells are used. In some embodiments mammalian cells are primate cells (human cells or non-human primate cells), rodent (e.g., mouse, rat, rabbit, hamster) cells, canine, feline, bovine, or other mammalian cells. In some embodiments avian cells are used. In some embodiments, the T cells are tumor-specific T cells.

In some embodiments, the T cell is a αβT cell, a cytotoxic T lymphocyte (CTL), a regulatory T cell, a natural killer T (NKT) cell, a Th17 cell, a γδT cell, or any combination thereof. In some embodiments, the T cell is an autologous cell. In some embodiments, the T cell is not an autologous cell. In some embodiments, the T cell is of the same species of a subject. In some embodiments, the T cell is of a species that is different than the species of a subject.

In certain embodiments a modified T cell is engineered to comprise a dual targeting CAR. In some aspects the dual targeting CAR has split co-stimulatory signal and a single CAR-CD3ζ domain. In some aspects, the modified T cell co-stimulates CD28 and 4-1BB. In some aspects, the modified T cell expresses GD2 and B7-H3. In certain aspects, the modified T cell comprises a GD2.28ζ.CAR/B7-H3.BB.CAR.

The modified T cells described herein exhibit one or more features. Non-limiting examples of the features of the modified T cells include dual antigen specificity and co-stimulation, killing activity and cytokine release of T cells via the GD2.28ζ.CAR or B7-H3.BB.CAR, increased IFN-γ and IL-2 release (as compared to a control cell), higher basal levels of TCR activation signaling (as compared to a control cell), enhanced phosphorylation of the CAR-CD3ζ chain and downstream signaling kinases (e.g., ERK and Akt), enrichment in cell cycle pathways (e.g., 5 days upon removal from antigen stimulation), enrichment in TCR signaling pathways (e.g., 5 days upon removal from antigen stimulation), elevated glycolytic activity (as compared to a control cell at day 0 and day 5 post-stimulation), controls tumor growth upon tumor re-challenge (as compared to a control cell), promotes enhanced tumor control and improved survival (as compared to a control cell), increased anti-tumor activity (e.g., under stress conditions), and combinations thereof. In some embodiments, the modified T cell exhibits dual antigen specificity and co-stimulation. In some embodiments, the modified T cell exhibits killing activity and cytokine release of T cells via the GD2.28ζ.CAR or B7-H3.BB.CAR. In some embodiments, the modified T cell exhibits increased IFN-γ and IL-2 release, as compared to a control cell. In some embodiments, the modified T cell exhibits higher basal levels of TCR activation signaling, as compared to a control cell. In some embodiments, the modified T cell exhibits enhanced phosphorylation of the CAR-CD3ζ and downstream signaling kinases (e.g., ERK and Akt). In some embodiments, the modified T cell exhibits enrichment in cell cycle pathways (e.g., day 5 upon removal from antigen stimulation). In some embodiments, the modified T cell exhibits enrichment in TCR signaling pathways (e.g., day 5 upon removal from antigen stimulation). In some embodiments, the modified T cell exhibits elevated glycolytic activity, as compared to a control cell (e.g., day 0 and day 5 post-stimulation). In some embodiments, the modified T cell controls tumor growth upon tumor re-challenge, as compared to a control cell. In some embodiments, the modified T cell promotes enhanced tumor control and improved survival, as compared to a control cell. In some embodiments, the modified T cell exhibits increased anti-tumor activity (e.g., under stress conditions).

In some embodiments T cells are isolated from a mammal and genetically modified (i.e., transduced or transfected in vitro) with the dual targeting CAR having a split co-stimulatory signal and a single CAR-CD3ζ domain. In some embodiments, a T cell can be transduced with a viral vector or transfected with a plasmid or nucleic acid construct. In some embodiments, the modified T cell is a tumor specific T cell that is transduced with a retroviral supernatant comprising a GD2.28ζ.CAR/B7-H3.BB.CAR.

For administration to a subject, modified T cells produced by the methods as disclosed herein can be administered to a subject, for example in pharmaceutically acceptable compositions. These pharmaceutically acceptable compositions comprise a therapeutically-effective amount of modified T cells as described above, formulated together with one or more pharmaceutically acceptable carriers (additives) and/or diluents. In some embodiments the pharmaceutical compositions comprising the modified T cells further include diluents and/or other components and/or other cytokines and/or cell populations.

As described herein, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. In some embodiments direct administration to a tumor and/or a body cavity, orifice, and/or tissue containing a tumor may be desired.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Methods of Treatment

Disclosed herein are methods of treating or preventing a cancer in a subject in need thereof. In some embodiments the method includes administering a modified T cell comprising a dual targeting CAR with split co-stimulatory signal and a single CAR-CD3ζ domain, as described herein. In some embodiments the method includes administering a therapeutically effective amount of modified T cells comprising a dual targeting CAR with split co-stimulatory signal and a single CAR-CD3ζ domain.

Also disclosed herein are methods of generating a population of modified T cells in a subject (e.g., a subject diagnosed with cancer and/or otherwise in need thereof). In some embodiments, the method includes administering to a subject a T cell modified to comprise a dual targeting CAR with split co-stimulatory signal and a single CAR-CD3ζ domain. In some aspects, the population of modified T cells persists in the subject for a period of time following administration to the subject (e.g., at least one week, one month, two months, three months, four months, five months, six months, nine months, one year, two years, five years, etc.). In some aspects, the population of modified T cells persists in the subject for a period of three months to nine months, and in certain aspects for a period of six months, following administration to the subject.

In some embodiment, the cells described herein, e.g. modified T cells are transplantable, e.g., modified T cells can be administered to a subject. In some embodiments, the subject who is administered modified T cells is the same subject from whom the pre-modified T cells was obtained (e.g. for autologous cell therapy). In some embodiments, the subject is a different subject. In some embodiments, a subject is suffering from cancer, or is a normal subject. For example, the modified T cells for transplantation can be a form suitable for transplantation.

The method can further include administering the modified T cells to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism.

A composition comprising modified T cells can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

As used herein, the term “administer” refers to the placement of a composition into a subject by a method or route which results in at least partial localization of the composition at a desired site such that desired effect is produced. Routes of administration suitable for the methods of the invention include both local and systemic administration. Generally, local administration results in more of the administered modified T cells being delivered to a specific location as compared to the entire body of the subject, whereas, systemic administration results in delivery of the modified T cells to essentially the entire body of the subject.

In the context of administering modified T cells, the term “administering” also include transplantation of such cells in a subject. As used herein, the term “transplantation” refers to the process of implanting or transferring at least one cell to a subject. The term “transplantation” includes, e.g., autotransplantation (removal and transfer of cell(s) from one location on a patient to the same or another location on the same patient), allotransplantation (transplantation between members of the same species), and xenotransplantation (transplantations between members of different species). A skilled artisan is well aware of methods for implanting or transplantation of cells for treating cancer, which are amenable to the present invention.

Modified T cells or compositions comprising the same can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. In addition, the methods and compositions described herein can be used to treat domesticated animals and/or pets.

In some embodiments a subject is deemed “at risk” of having or developing cancer or recurrence of cancer. Whether a subject is at risk of having or developing cancer or having a recurrence of cancer is a determination that may be within the discretion of the skilled practitioner caring for the subject. Any suitable diagnostic test and/or criteria can be used. For example, a subject may be considered “at risk” of having or developing cancer if (i) the subject has a mutation, genetic polymorphism, gene or protein expression profile, and/or presence of particular substances in the blood, associated with increased risk of developing or having cancer relative to other members of the general population not having mutation or genetic polymorphism; (ii) the subject has one or more risk factors such as having a family history of cancer, having been exposed to a carcinogen or tumor-promoting agent or condition, e.g., asbestos, tobacco smoke, aflatoxin, radiation, chronic infection/inflammation, etc., advanced age; (iii) the subject has one or more symptoms of cancer, (iv) the subject has a medical condition that is known to increase the likelihood of cancer, etc.

As used herein, the type of cancer is not limited. The term “cancer” as used herein is defined as a hyperproliferation of cells whose unique trait-loss of normal controls-results in unregulated growth, lack of differentiation, local tissue invasion, and metastasis. With respect to the inventive methods, the cancer can be any cancer, including any of acute lymphocytic cancer, acute myeloid leukemia, adenocarcinoma, alveolar rhabdomyosarcoma, anal cancer, angiosarcoma, B cell lymphoma, basal cell carcinoma, bladder cancer, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, colorectal cancer, esophageal cancer, cervical cancer, endometrial cancer, fibrosarcoma, gastrointestinal carcinoid tumor, hematopoietic neoplasias, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, sarcoma, skin cancer, small intestine cancer, soft tissue cancer, solid tumors, squamous cell carcinoma, stomach cancer, T cell lymphoma, testicular cancer, thymoma, thyroid cancer, ureter cancer, urinary bladder cancer, and uterine cancer. In certain aspects, the cancer is a neuroblastoma. As used herein, the term “tumor” refers to an abnormal growth of cells or tissues of the malignant type, unless otherwise specifically indicated and does not include a benign type tissue.

As used herein, the term “treating” and “treatment” refers to administering to a subject an effective amount of modified T cells altered ex vivo according to the methods described herein so that the subject has a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a disorder associated with expression of a polynucleotide sequence, as well as those likely to develop such a disorder due to genetic susceptibility or other factors.

By “treatment,” “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

The dosage, administration schedule and method of administering the modified T cells are not limited. The dosage will depend upon a variety of factors including other treatment, the number of doses and the individual patient parameters including age, physical condition, size and weight. These are factors well known to those of ordinary skill in the art and can be addressed with no more than routine experimentation. In some embodiments, a maximum tolerated dose may be used, that is, the highest safe and tolerable dose according to sound medical judgment. In some embodiments, a pharmaceutical composition comprising the modified T cells can be administered at a dosage of about 10³ to about 10¹⁰ cells/kg body weight, and in some embodiments, the dosage can be from about 10⁵ to about 10⁶ cells/kg body weight, including all integer values (e.g., 10⁴, 10⁵, 10⁶, 10⁷,108, 109) within those ranges.

The dose used may be the maximal tolerated dose or a sub-therapeutic dose or any dose therebetween. In some embodiments modified T cells are administered in combination with one or more agents. In some embodiments, the modified T cells and/or the one or more agents are administered according to a defined administration schedule. Multiple doses are contemplated. In some embodiments, when the modified T cells and one or more agents are administered in combination, a sub-therapeutic dosage of one or more of the agents may be used. A “sub-therapeutic dose” as used herein refers to a dosage which is less than that dosage which would produce a therapeutic result in the subject if administered in the absence of the other agent. In some aspects, a sub-therapeutic dose of an anticancer agent is one which would not produce a useful therapeutic result in the subject in the absence of the administration of the modified T cells described herein. Therapeutic doses of anticancer agents are well known in the field of medicine for the treatment of cancer.

As used herein, pharmaceutical compositions comprise one or more agents or compositions that have therapeutic utility, and a pharmaceutically acceptable carrier, e.g., a carrier that facilitates delivery of agents or compositions. Agents and pharmaceutical compositions disclosed herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or as an aerosol. Depending upon the type of condition (e.g., cancer) to be treated, compounds of the invention may, for example, be inhaled, ingested or administered by systemic routes. Thus, a variety of administration modes, or routes, are available. The particular mode selected will typically depend on factors such as the particular compound selected, the particular condition being treated and the dosage required for therapeutic efficacy. The methods described herein, generally speaking, may be practiced using any mode of administration that is medically acceptable, meaning any mode that produces acceptable levels of efficacy without causing clinically unacceptable adverse effects.

Preferred modes of administration are parenteral and oral routes. The term “parenteral” includes subcutaneous, intravenous, intramuscular, intraperitoneal, and intrasternal injection, or infusion techniques. In some embodiments, inhaled medications are of particular use because of the direct delivery to the lung, for example in lung cancer patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. In some embodiments agents are delivered by pulmonary aerosol. Other appropriate routes will be apparent to one of ordinary skill in the art.

Toxicity and therapeutic efficacy of administration of compositions comprising modified T cells can be determined by standard pharmaceutical procedures in cell cultures or experimental animals, e.g., for determining the LD50 (the dose lethal to 50% of the population) and the ED50 (the dose therapeutically effective in 50% of the population). Compositions comprising modified T cells that exhibit large therapeutic indices are preferred.

The amount of a composition comprising modified T cells can be tested using several well-established animal models.

In some embodiments, data obtained from the cell culture assays and in animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose of a composition comprising modified T cells can also be estimated initially from cell culture assays. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect of the invention, the methods provide use of an isolated population of modified T cells. In one embodiment of the invention, an isolated population of modified T cells as disclosed herein may be used for the production of a pharmaceutical composition, for the use in transplantation into subjects in need of treatment, e.g. a subject that has, or is at risk of developing cancer. Examples include subjects with melanoma or pancreatic cancer. In some embodiments, an isolated population of modified T cells as disclosed herein may be autologous and/or allogeneic. In some embodiments, the subject is a mammal, and in other embodiments the mammal is a human.

One embodiment of the invention relates to a method of treating cancer in a subject comprising administering an effective amount of a composition comprising modified T cells as disclosed herein to a subject with cancer. Other embodiments relate to a method of treating a neuroblastoma in a subject comprising administering an effective amount of a composition comprising modified T cells as disclosed herein to a subject with a neuroblastoma.

In some embodiments, the modified T cells as disclosed herein are administered to a subject having cancer in combination with a second therapeutic treatment (e.g., chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytotoxin, fludaribine, cyclosporin, FK506, rapamycin, mycophenolic acid, steroids, FR901228, cytokines, and/or irradiation).

In some embodiments, the modified T cells are administered to a patient in conjunction with (e.g., before, concurrently and/or following) bone marrow transplantation, T cell ablative therapy using either chemotherapy agents such as fludarabine, external-beam radiation therapy (XRT), cyclophosphamide, or antibodies such as OKT3 or CAMPATH. In another embodiment, the modified T cells are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects can receive an infusion of the expanded modified T cells. In an additional embodiment, expanded cells can be administered before and/or following surgery.

In the treatment of cancers or tumors the modified T cells may optionally be administered in conjunction with other, different, cytotoxic agents such as chemotherapeutic or antineoplastic compounds or radiation therapy useful in the treatment of the disorders or conditions described herein (e.g., chemotherapeutics or antineoplastic compounds). The other compounds may be administered prior to, concurrently and/or after administration of the modified T cells. As used herein, the word “concurrently” means sufficiently close in time to produce a combined effect (that is, concurrently may be simultaneously, or it may be two or more administrations occurring before or after each other)

As used herein, the phrase “radiation therapy” includes, but is not limited to, x-rays or gamma rays which are delivered from either an externally applied source such as a beam or by implantation of small radioactive sources.

Nonlimiting examples of suitable chemotherapeutic agents which may be administered with the modified T cells as described herein include daunomycin, cisplatin, verapamil, cytosine arabinoside, aminopterin, democolcine, tamoxifen, Actinomycin D, Alkylating agents (including, without limitation, nitrogen mustards, ethylenimine derivatives, alkyl sulfonates, nitrosoureas and triazenes): Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide; Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine, Natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as Taxol®), Mithramycin, Deoxyco-formycin, Mitomycin-C, L-Asparaginase, Interferons (especially IFN-α), Etoposide, and Teniposide; Other anti-proliferative cytotoxic agents are navelbene, CPT-11, anastrazole, letrazole, capecitabine, reloxafine, cyclophosphamide, ifosamide, and droloxafine. Additional anti-proliferative cytotoxic agents include, but are not limited to, melphalan, hexamethyl melamine, thiotepa, cytarabin, idatrexate, trimetrexate, dacarbazine, L-asparaginase, camptothecin, topotecan, bicalutamide, flutamide, leuprolide, pyridobenzoindole derivatives, interferons, and interleukins. Preferred classes of antiproliferative cytotoxic agents are the EGFR inhibitors, Her-2 inhibitors, CDK inhibitors, and Herceptin® (trastuzumab). (see, e.g., U.S. Pat. Nos. 6,537,988; 6,420,377). Such compounds may be given in accordance with techniques currently known for the administration thereof.

In some embodiments, the modified T cells as disclosed herein may be administered in any physiologically acceptable excipient, where the modified T cells may find an appropriate site for replication, proliferation, and/or engraftment. In some embodiments, the modified T cells as disclosed herein can be introduced by injection, catheter, or the like. In some embodiments, the modified T cells as disclosed herein can be frozen at liquid nitrogen temperatures and stored for long periods of time, being capable of use on thawing. If frozen, the modified T cells will usually be stored in a 10% DMSO, 50% FCS, 40% RPMI 1640 medium. Once thawed, the cells may be expanded by use of growth factors and/or feeder cells associated with culturing T cells.

In some embodiments, the modified T cells as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising the modified T cells as disclosed herein will be adapted in accordance with the route and device used for administration. In some embodiments, a composition comprising the modified T cells can also comprise or be accompanied with one or more other ingredients that facilitate the engraftment or functional mobilization of the modified T cells. Suitable ingredients include matrix proteins that support or promote adhesion of the modified T cells, or complementary cell types. In another embodiment, the composition may comprise resorbable or biodegradable matrix scaffolds.

In some embodiments, the modified T cells can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. Modified T cells can be administered to a subject at the following locations: clinic, clinical office, emergency department, hospital ward, intensive care unit, operating room, catheterization suites, and radiologic suites.

In other embodiments, the modified T cells are stored for later implantation/infusion. The modified T cells may be divided into more than one aliquot or unit such that a portion of the modified T cells are retained for later application while part is applied immediately to the subject. Moderate to long-term storage of all or part of the cells in a cell bank is also within the scope of this invention, as disclosed in U.S. Patent Publication No. 2003/0054331 and Patent Publication No. WO 03/024215, and is incorporated by reference in their entireties. At the end of processing, the concentrated cells may be loaded into a delivery device, such as a syringe, for placement into the recipient by any means known to one of ordinary skill in the art.

Pharmaceutical compositions comprising effective amounts of modified T cells are also contemplated by the present invention. These compositions comprise an effective number of modified T cells, optionally, in combination with a pharmaceutically acceptable carrier, additive or excipient. Systemic administration of modified T cells to the subject may be preferred in certain indications, whereas direct administration at the site of or in proximity a tumor may be preferred in other indications.

In some embodiments, modified T cells can optionally be packaged in a suitable container with written instructions for a desired purpose, such as the reconstitution or thawing (if frozen) of the modified T cells prior to administration to a subject.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

As used herein “A and/or B”, where A and B are different claim terms, generally means at least one of A, B, or both A and B. For example, one sequence which is complementary to and/or hybridizes to another sequence includes (i) one sequence which is complementary to the other sequence even though the one sequence may not necessarily hybridize to the other sequence under all conditions, (ii) one sequence which hybridizes to the other sequence even if the one sequence is not perfectly complementary to the other sequence, and (iii) sequences which are both complementary to and hybridize to the other sequence.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

As used herein the term “comprising” or “comprises” is used in reference to compositions, methods, and respective component(s) thereof, that are essential to the invention, yet open to the inclusion of unspecified elements, whether essential or not. As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### Example 1

### Results

Single or dual antigen targeting CAR-T cells with single or dual CD28 or 4-1BB co-stimulation do not eradicate tumor in stress conditions

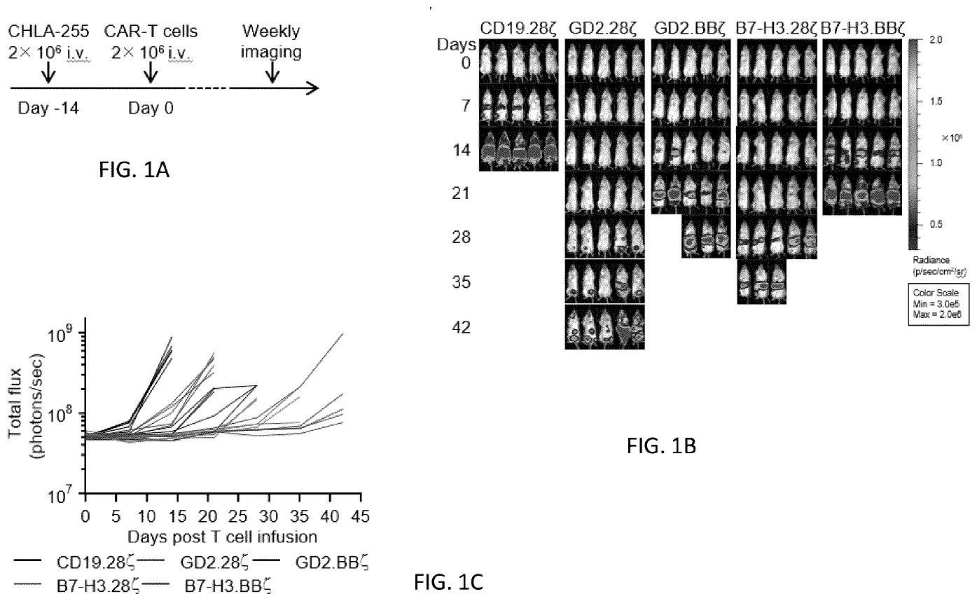

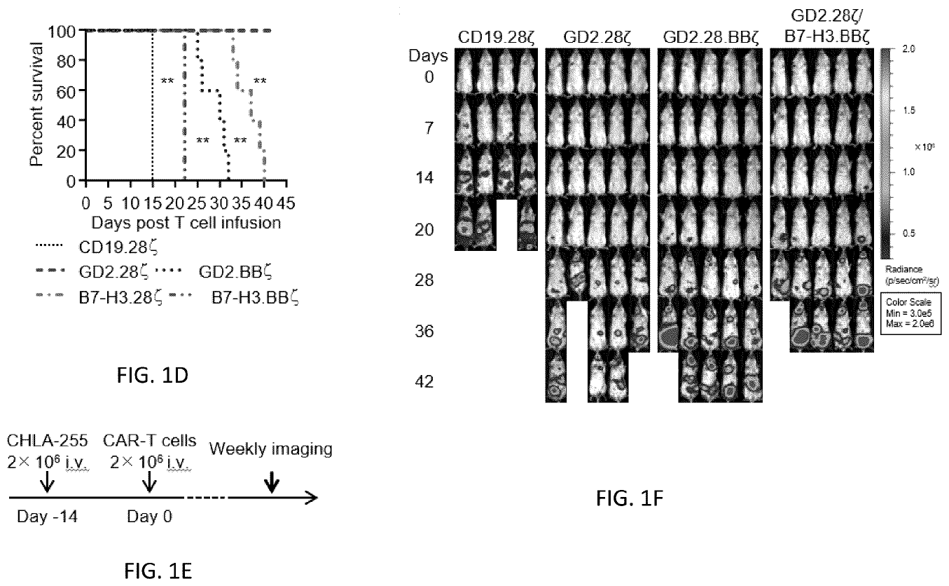

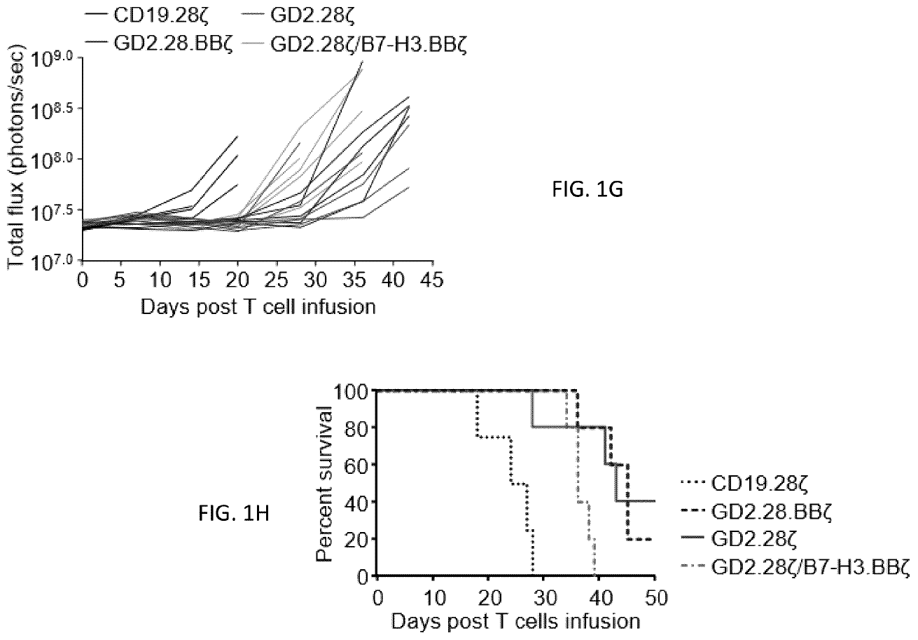

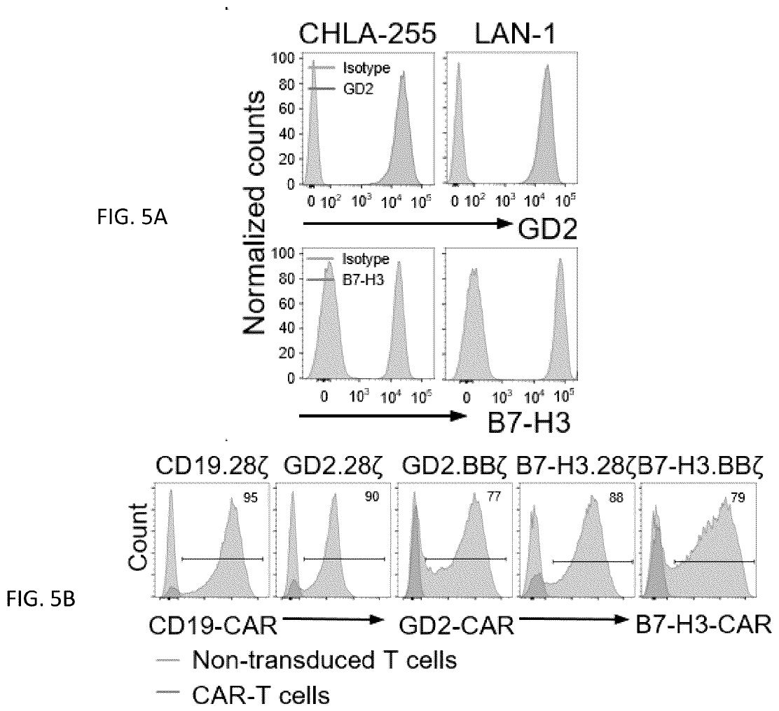

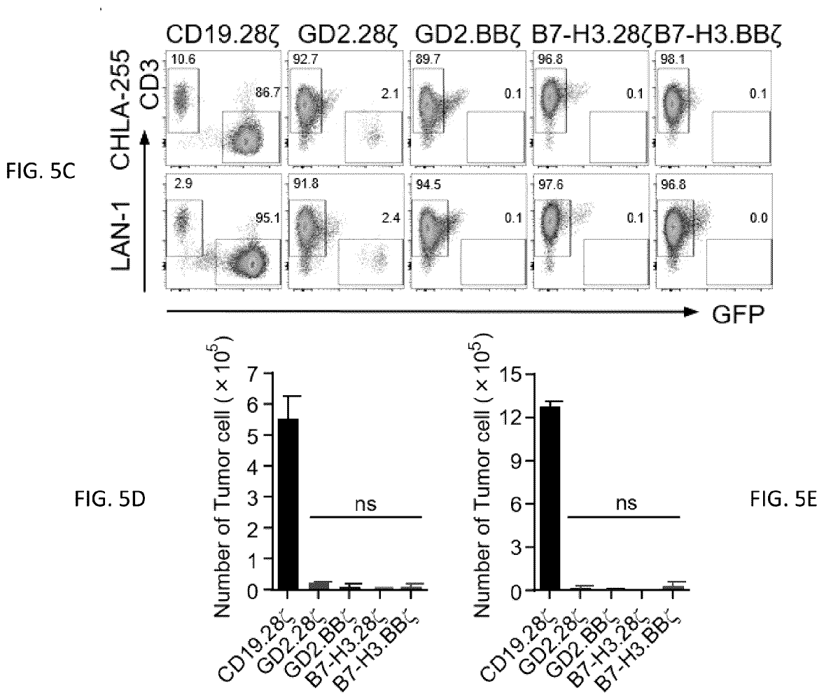

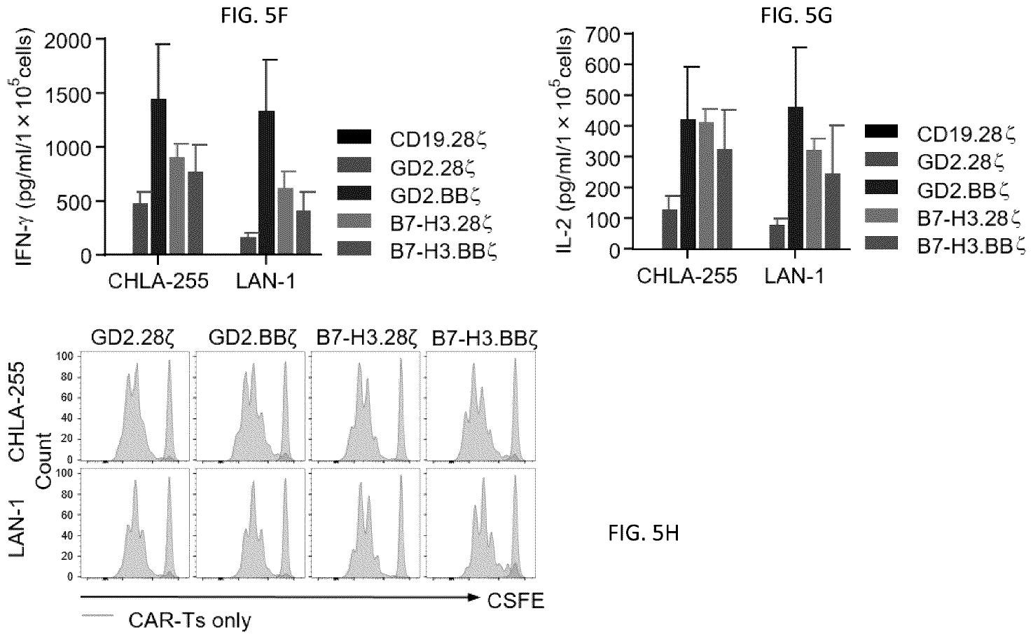

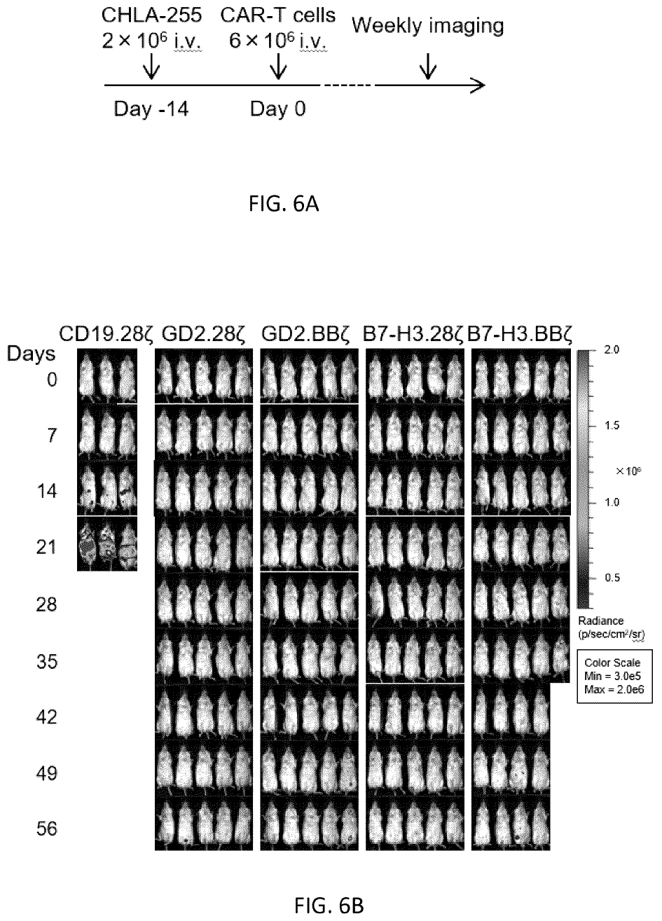

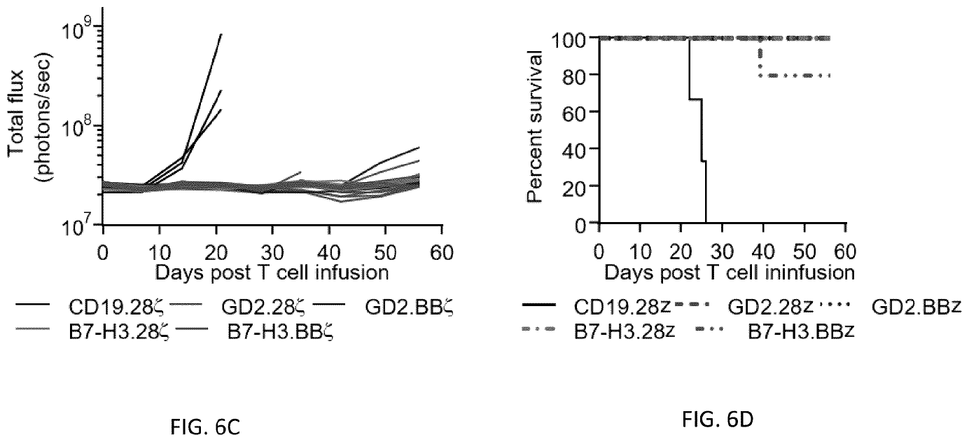

Neuroblastoma (NB) as a tumor model, and two tumor cell lines (CHLA-255 and LAN-1) that co-express two targetable antigens, GD2 and B7-H3 as assessed by flow cytometry (FIG. 5A) were used. T cells engineered to express GD2.28ζ.CAR, GD2.BBζ.CAR, B7-H3.28ζ.CAR, and B7-H3.BBζ.CAR effectively eliminated tumor cells in vitro without any significant differences (FIGS. 5C-5E). The cytolytic activity of CAR-T cells was corroborated by IFN-γ and IL-2 release in the culture supernatant (FIGS. 5F-5G) and by T cell proliferation in response to NB cell lines (FIG. 5H). GD2.28ζ.CAR, GD2.BBζ.CAR, B7-H3.28ζ.CAR, and B7-H3.BBζ.CAR-T cells equally and effectively controlled tumor growth in NSG mice engrafted with CHLA-255 when a high dose of CAR-T cells was used (FIGS. 6A-6D). In sharp contrast, when CAR-T cells were used in stress conditions, such as those in which CHLA-255-bearing mice are treated with low doses of CAR-T cells (2×10⁶ CAR-T cells/mouse) (FIG. 1A), GD2.28ζ.CAR-T cells exhibited superior tumor control as compared to GD2.BBζ.CAR, B7-H3.28ζ.CAR, and B7-H3.BBζ.CAR-T cells (FIGS. 1B-1D).

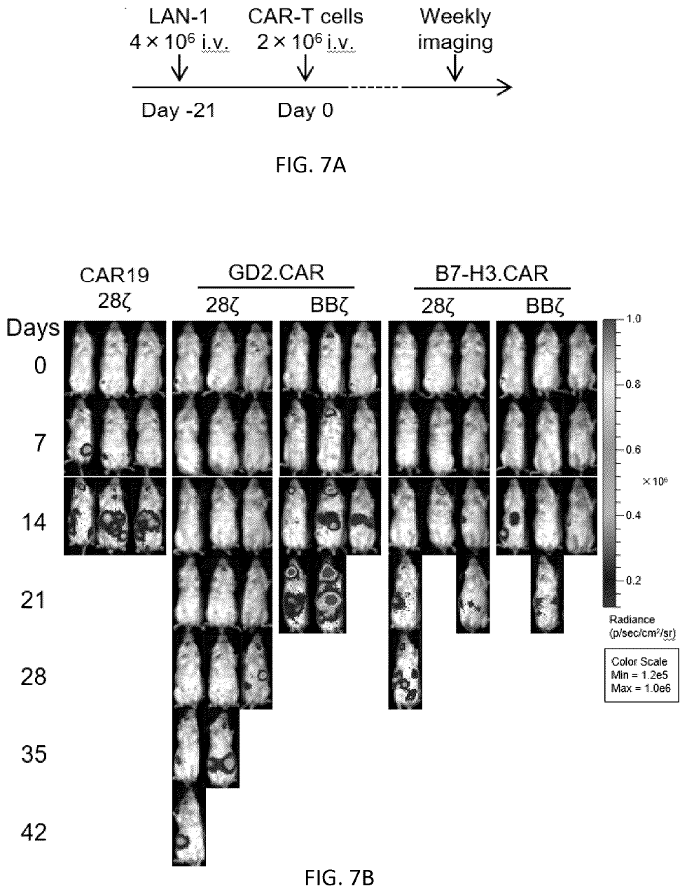

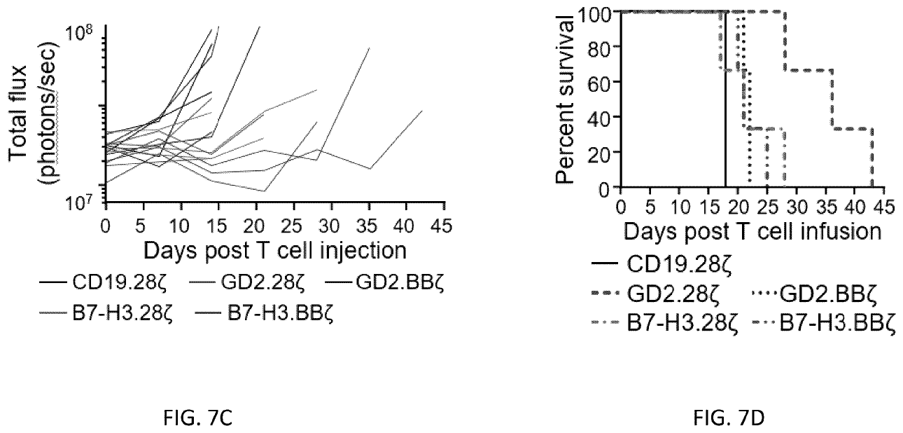

Similar results were observed in a second NB model in which mice were engrafted with LAN-1 cells (FIGS. 7A-7D).

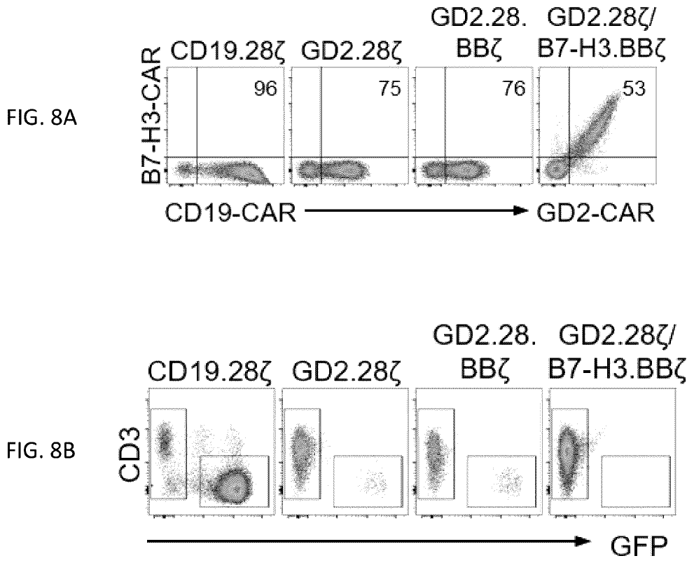

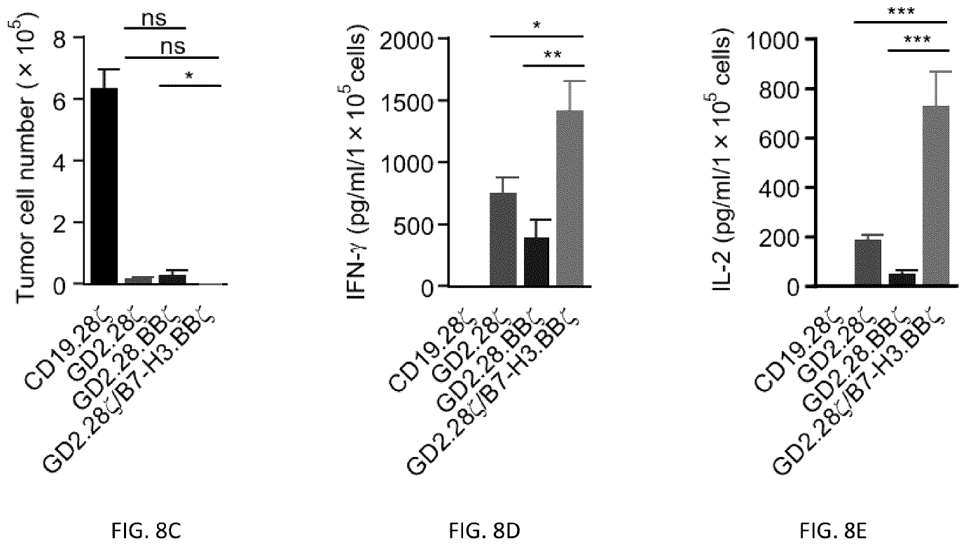

Since GD2.28ζ.CAR-T cells showed the most prominent antitumor activity, but did not fully eradicate the tumor at low doses, the addition of 4-1BB co-stimulation was assessed to determine if it would lead to tumor eradication as previously described¹¹. 4-1BB co-stimulation was provided in the form of a conventional 3^{rd} generation CAR (GD2.28.BBζ.CAR). In addition, a vector encoding simultaneously GD2.28ζ.CAR and B7-H3.BBζ.CAR (GD2.28ζ.CAR/B7-H3.BBζ.CAR) was constructed to provide both dual specificity and dual co-stimulation. Both in vitro (FIGS. 8A-8E) and in vivo (FIGS. 1E-1H) experiments showed that 3^{rd} generation GD2.28.BBζ.CAR-T cells and double CAR with split co-stimulation obtained with GD2.28ζ.CAR/B7-H3.BBζ.CAR-T cells did not show superior antitumor activity as compared to GD2.28ζ.CAR-T cells in stress conditions. Overall, these data demonstrate that GD2.28ζ.CAR-T cells are the most effective in controlling the tumor growth at low doses, but do not eradicate the tumor in stress conditions. Furthermore, 4-1BB co-stimulation provided either in the form of 3^{rd} generation CAR or dual CARs does not enhance the therapeutic effect.

Dual targeting CAR-T cells with split co-stimulation and shared CD3C domain have improved expansion, cytokine release and antitumor activity

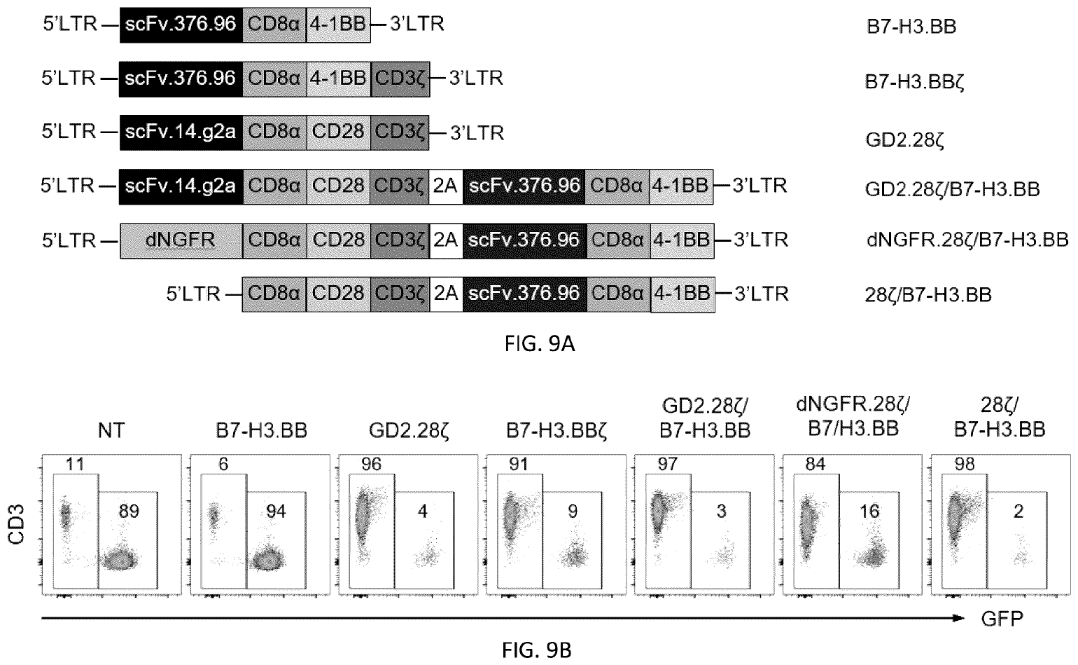

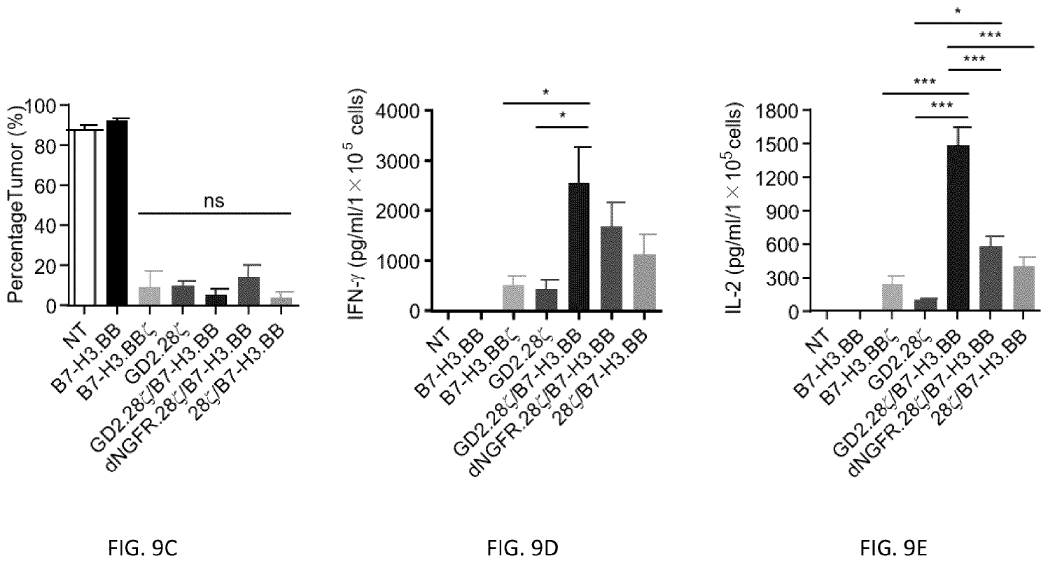

It was hypothesized that GD2.28ζ.CAR/B7-H3.BBζ.CAR-T cells may receive an excessive CAR-CD3ζ signaling that compromises the beneficial effects of the dual co-stimulation and dual targeting. Thus a series of dual CARs encoded in a single retroviral vector were generated to assess if a shared CAR-CD3ζ chain is sufficient to provide dual antigen specificity and co-stimulation (FIG. 9A).

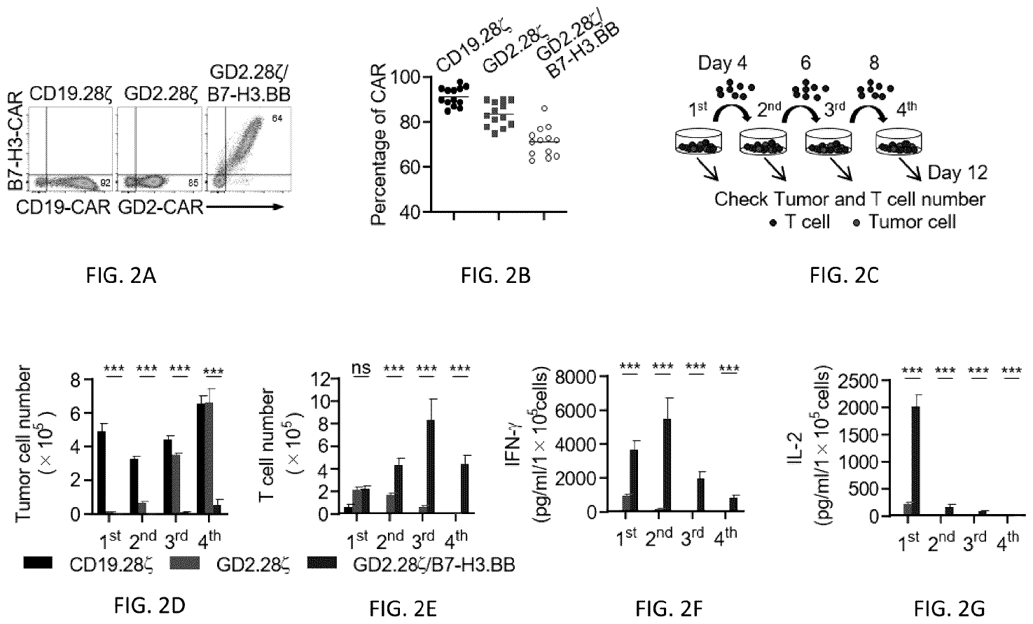

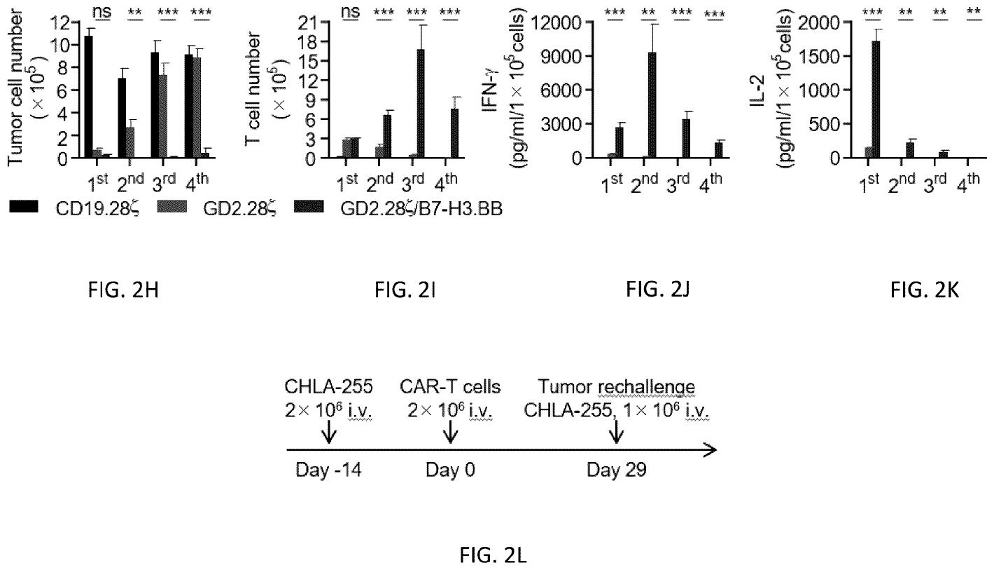

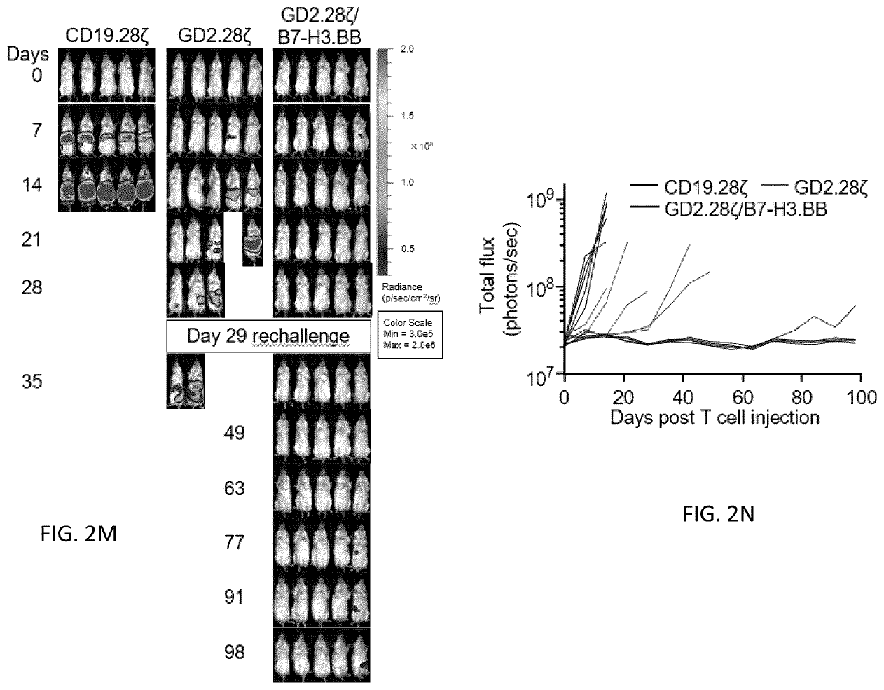

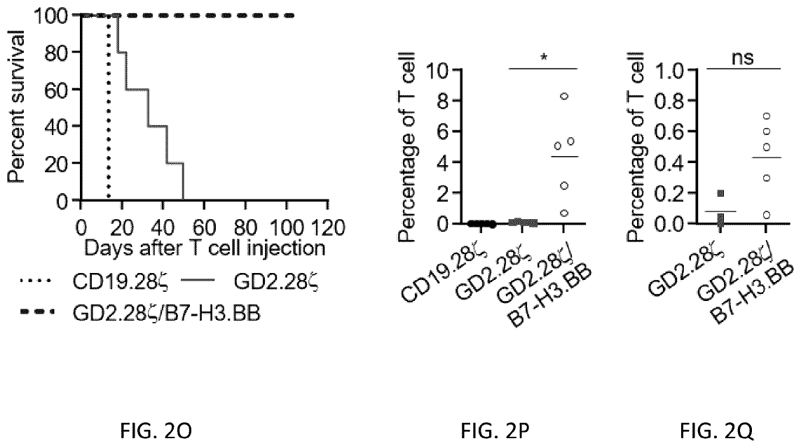

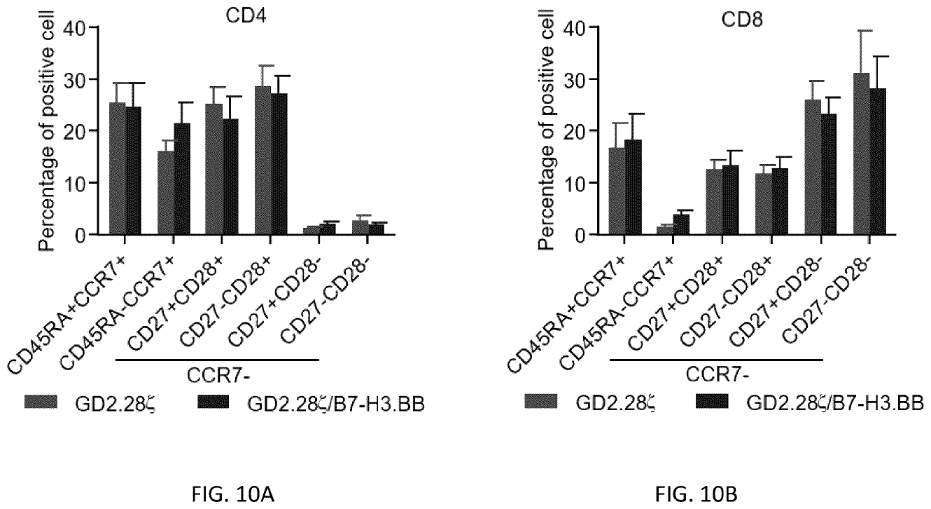

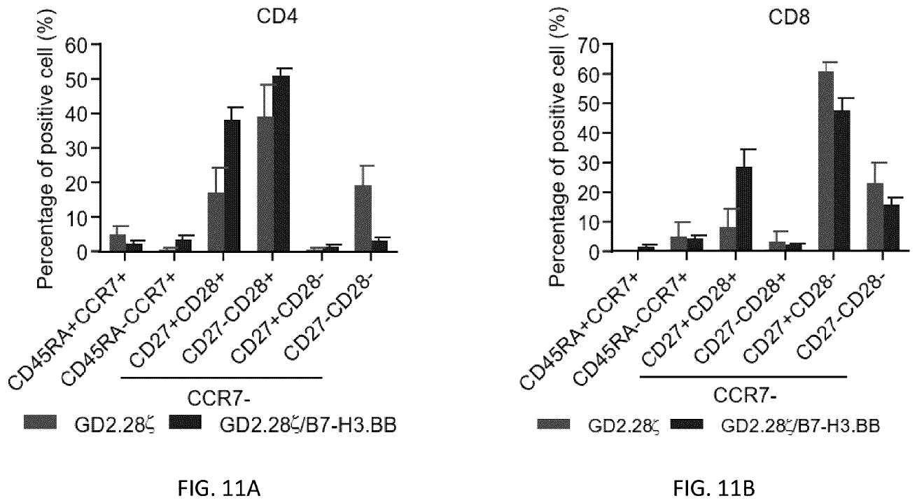

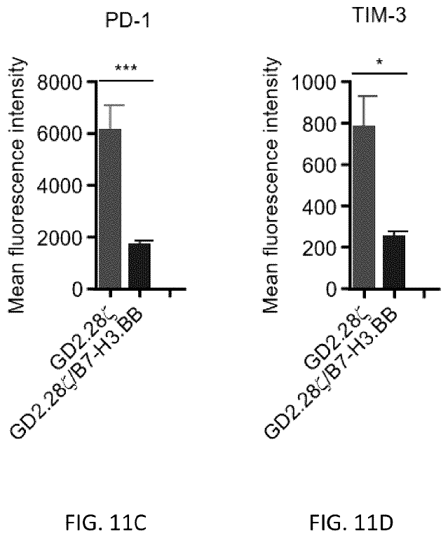

One single CAR-CD3ζ domain incorporated in the GD2.28ζ.CAR co-expressed with the B7-H3.CAR with 4-1BB, but without the CD3ζ chain (GD2.28ζ.CAR/B7-H3.BB.CAR) provided killing activity and cytokine release of T cells via either the GD2.28ζ.CAR or B7-H3.BB.CAR engagement (FIGS. 9B-9C). In vitro and in vivo T cells expressing the GD2.28ζ.CAR or the GD2.28ζ.CAR/B7-H3.BB.CAR were then compared. All CARs expressed well in T cells (FIGS. 2A-2B), and no modifications in cell subset compositions were observed (FIGS. 9D-9E). In repetitive multi-round co-culture experiments with NB tumor cells (FIG. 2C), T cells expressing the GD2.28ζ.CAR/B7-H3.BB.CAR continued to eliminate NB cells at the 4^{th} round of co-culture as compared to T cells expressing the GD2.28ζ.CAR alone (FIG. 2D, FIG. 2H). In addition, T cells expressing the GD2.28ζ.CAR/B7-H3.BB.CAR showed the highest T cell counts (FIG. 2E, FIG. 2I) and the highest IFN-y and IL-2 release (FIGS. 2F-2G, FIGS. 2J-K) at the 3^{rd} and 4^{t} round of co-cultures. In the CHLA-255-bearing NSG mice, T cells expressing the GD2.28ζ.CAR/B7-H3.BB.CAR not only showed superior antitumor activity to eliminate the primary tumor in stress conditions, but also controlled tumor growth upon tumor re-challenge leading to improved survival (FIGS. 2L-20). At day 14, mice treated with T cells expressing the GD2.28ζ.CAR/B7-H3.BB.CAR showed the highest frequency of circulating T cells (FIG. 2P), and at day 28 they continued to have a trend of higher circulating T cells (FIG. 2Q). T cells expressing GD2.28ζ.CAR/B7-H3.BB.CAR were enriched in CD27+CD28⁺ cells in both CD4⁺ and CD8⁺ T cells (FIGS. 11A-11B), and showed low expression of PD-1 and TIM3 (FIGS. 11C-11D). Overall, these data indicate that dual CAR targeting, split signal and one single CD3ζ domain promote sustained survival and antitumor effects of CAR-T cells.

Dual targeting, split signaling and one single CD3C endodomain promote sustained T cell activation profile and metabolic fitness

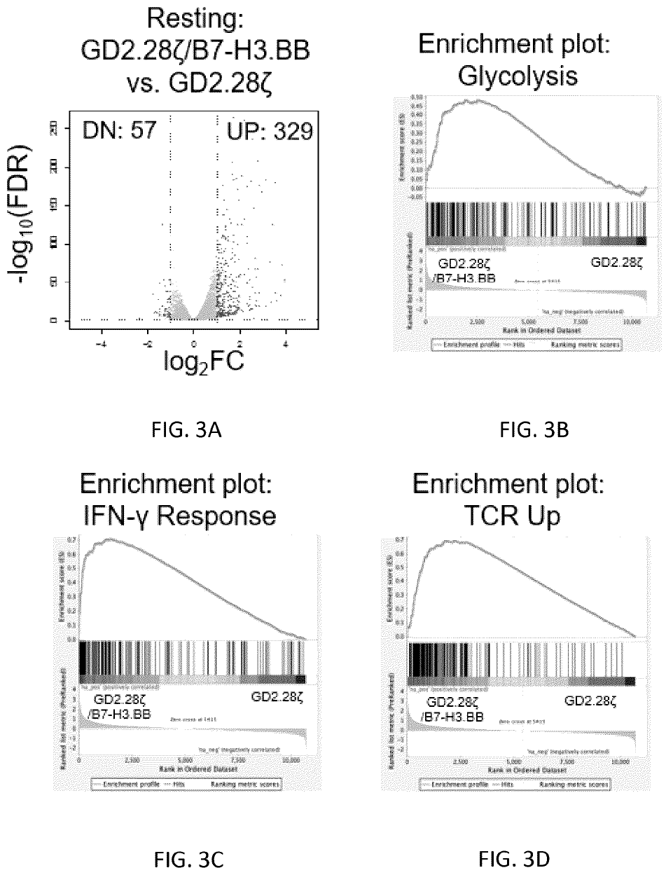

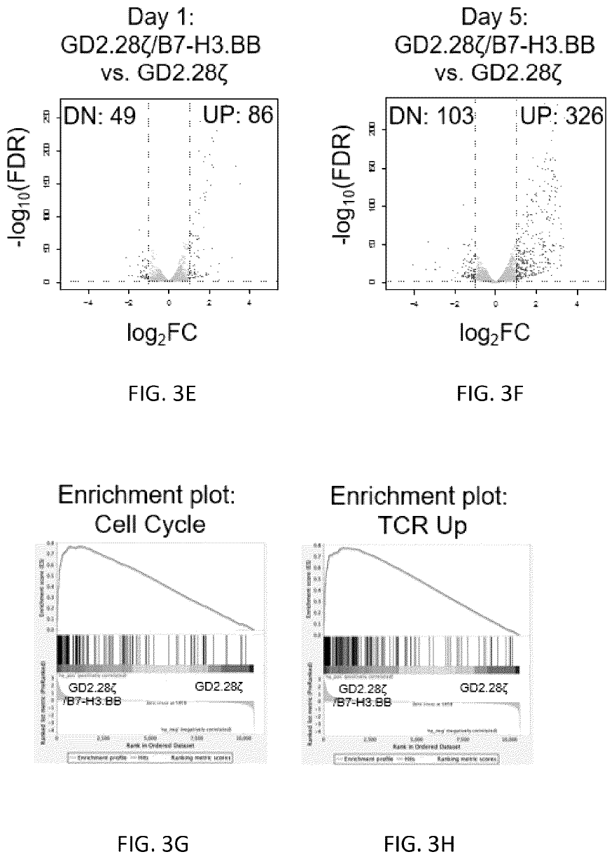

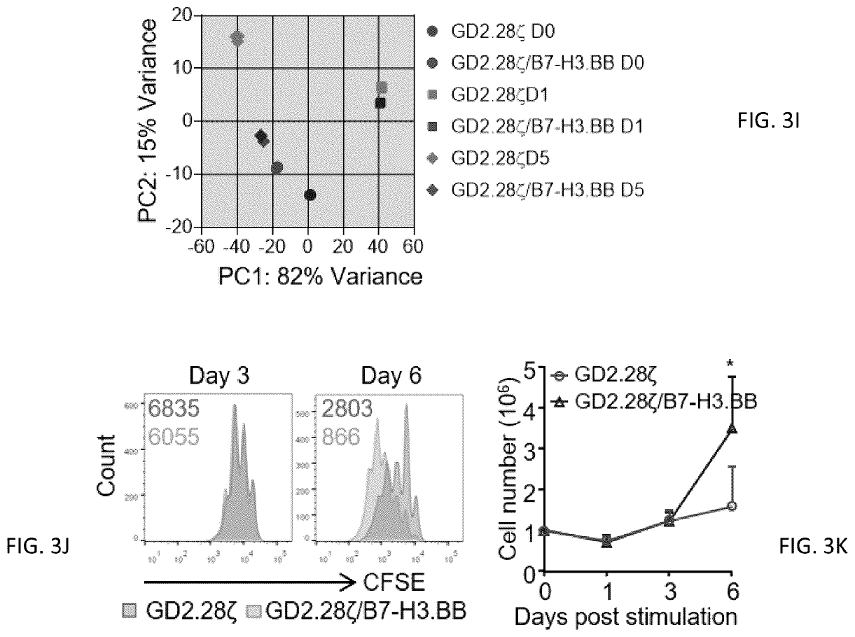

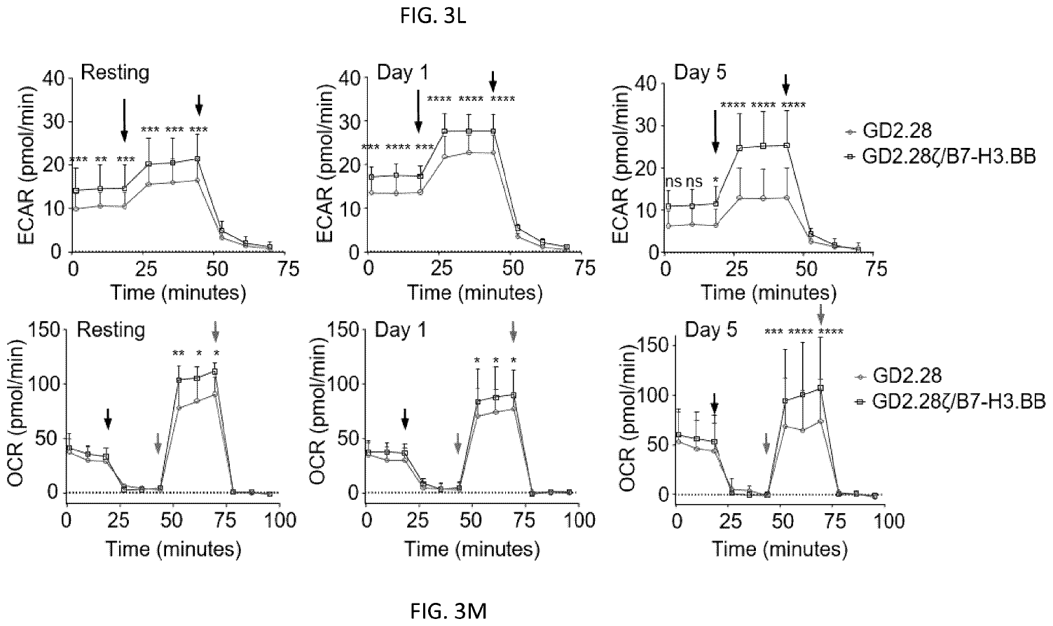

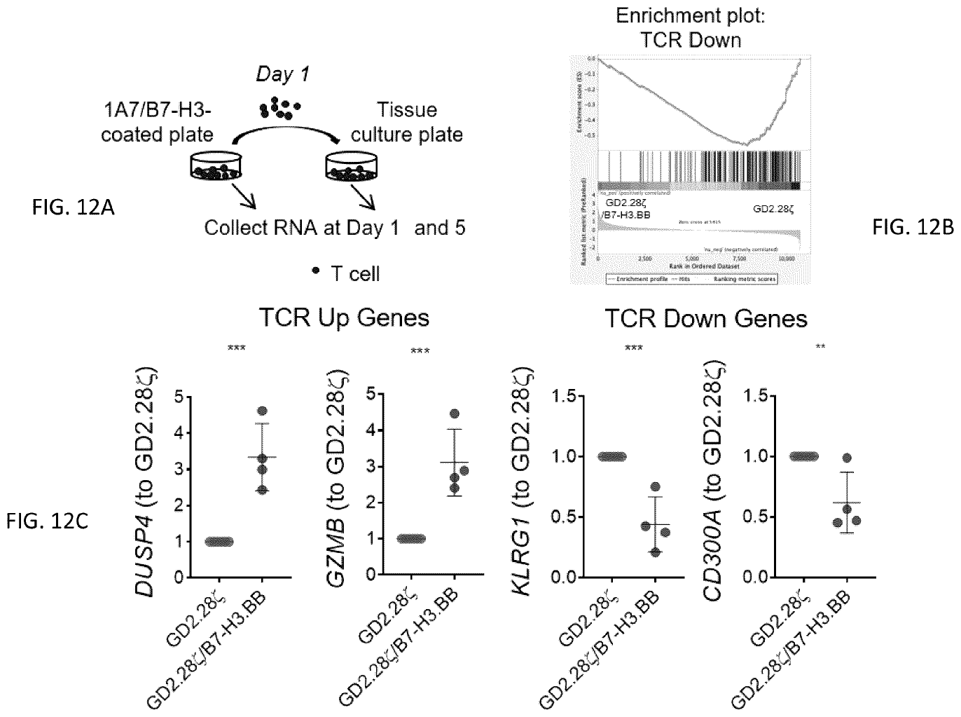

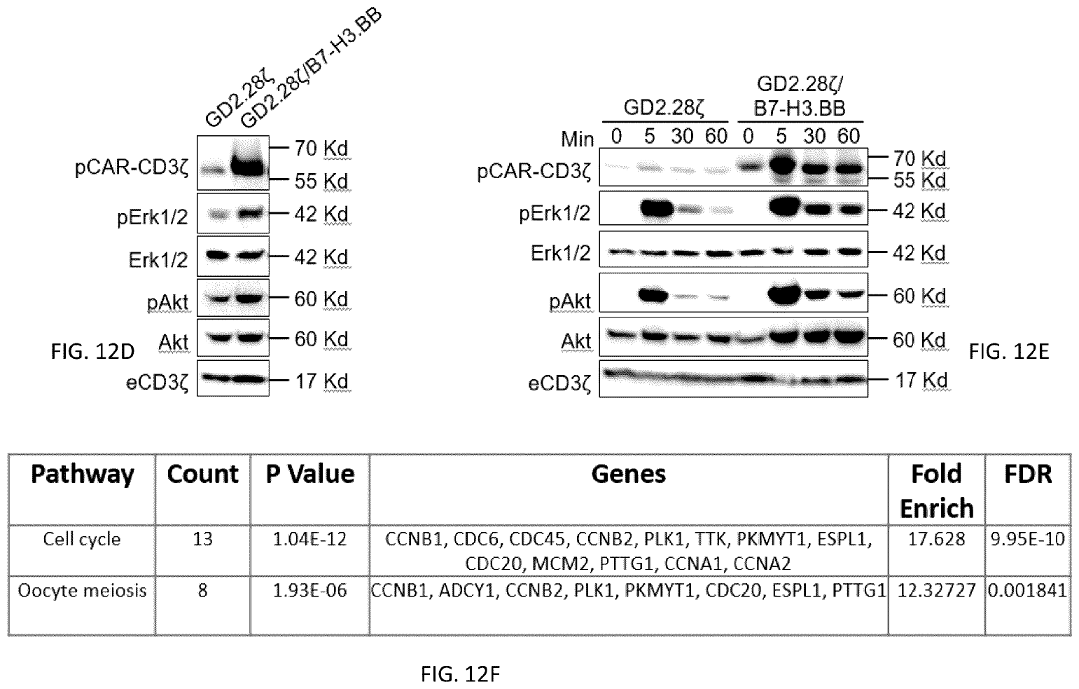

RNASeq analysis was performed to investigate how the addition of B7-H3.BB.CAR to the GD2.28ζ.CAR enhances antitumor activity and persistence of CAR-T cells. In the absence of CAR engagement, it was found that T cells carrying the GD2.28ζ.CAR/B7-H3.BB.CAR showed different gene expression pattern as compared to those expressing the GD2.28ζ.CAR (FIG. 3A). Gene set enrichment analysis (GSEA) showed that glycolytic pathways and IFN-γ signaling are elevated in T cells expressing the GD2.28ζ.CAR/B7-H3.BB.CAR (FIGS. 3B-3C). Since both glycolytic and IFN-γ pathways are activated by TCR signaling, the data set was tested using T cell activation gene sets. The transcriptome of T cells expressing the GD2.28ζ.CAR/B7-H3.BB.CAR was highly enriched with genes upregulated upon T cell activation as compared to GD2.28ζ.CAR, while genes downregulated upon T cell activation are enriched in GD2.28ζ.CAR expressing cells (FIG. 3D and FIGS. 12A-12C). These data indicate that GD2.28ζ.CAR/B7-H3.BB.CAR-T cells have higher basal level of TCR activation signaling, which is paralleled by enhanced phosphorylation of the CAR-CD3ζ chain and downstream signaling kinases such as ERK and Akt (FIG. 12D). It was previously reported that basal CAR-CD3ζ phosphorylation in CAR-T cells encoding the CD28 endodomain primes them to stronger and sustained activation upon CAR cross-linking²⁸. Here it was observed that this effect is further amplified in T cells expressing the GD2.28ζ.CAR/B7-H3.BB.CAR as compared to T cells expressing the GD2.28ζ.CAR (FIG. 12E).

Despite dramatic differences in transcriptome at basal resting condition, initial CAR-mediated stimulation equally activated T cells expressing either GD2.28ζ.CAR/B7-H3.BB.CAR or GD2.28ζ.CAR as transcriptome profile of T cells with either CAR converged at day 1 (FIG. 3E). However, gene expression diverged between T cells expressing GD2.28ζ.CAR/B7-H3.BB.CAR and GD2.28ζ.CAR at day 5, upon removal from antigen stimulation (FIG. 3F) with T cells expressing the GD2.28ζ.CAR/B7-H3.BB.CAR showing an enrichment in pathways related to cell cycle and TCR signaling, suggesting sustained proliferation upon antigen removal (FIGS. 3G-3H).

Principle component analysis was performed to determine the relative relationship between the transcriptome of CAR-T cells at day 0, 1 and 5 (FIG. 3I). The variance of the transcriptome of the dataset is dominated by CAR activation, which is captured on PC1 (x-axis). On day 0 and day 5, T cells expressing GD2.28ζ.CAR/B7-H3.BB showed higher PC1 score when compared to those with GD2.28ζ.CAR, consistent with the finding of active TCR signaling. The transcriptome of CAR-T cells at day 5 does not mirror that at day 0, clustering to the left of day 0 CAR-T cells on PC1 axis, suggesting that these activated and rested T cells enter a “de-activated” status. Notably, T cells expressing GD2.28ζ.CAR/B7-H3.BB acquired additional transcriptome distinction from T cells expressing the GD2.28ζ.CAR, which is captured in PC2 (y-axis). When KEGG pathway analysis of the top 100 highest loading genes in PC2 was performed, the “Cell Cycle pathway” was found to be the most enriched (FIG. 12F). Consistent with the enrichment of the cell cycle related pathway, T cells expressing the GD2.28ζ.CAR/B7-H3.BB.CAR were found to show sustained T cell proliferation at day 6 post CAR crosslinking (FIG. 3J) and better expansion as indicated by almost two fold more T cell counts at day 6 as compared to T cells expressing the GD2.28ζ.CAR (FIG. 3K). On metabolic analyses, T cells expressing GD2.28ζ.CAR/B7-H3.BB.CAR showed elevated glycolytic activity as compared to those expressing the GD2.28ζ.CAR at day 0 and day 5 post-stimulation, while only modest differences were observed at day 1 when activation signaling is at its maximum for T cells expressing either GD2.28ζ.CAR/B7-H3.BB.CAR or GD2.28ζ.CAR (FIG. 3L). Remarkably, enhanced glycolysis observed in T cells expressing the GD2.28ζ.CAR/B7-H3.BB.CAR did not disrupt the previously described pro-OXPHOS metabolic profile sustained by 4-1BB endodomain²⁹, because these cells showed significantly increased oxygen consumption rate before or after CAR stimulation as compared to T cells expressing GD2.28ζ.CAR (FIG. 3M). Overall, transcriptional analysis, T cell signaling and metabolism suggest that dual targeting, split signal and one single CD3ζ domain maintain tonic TCR signaling and metabolic fitness in T cells and translate into potent and sustained antitumor activity.

Dual targeting, split signaling and one single CD3C endodomain prevent antigen escape

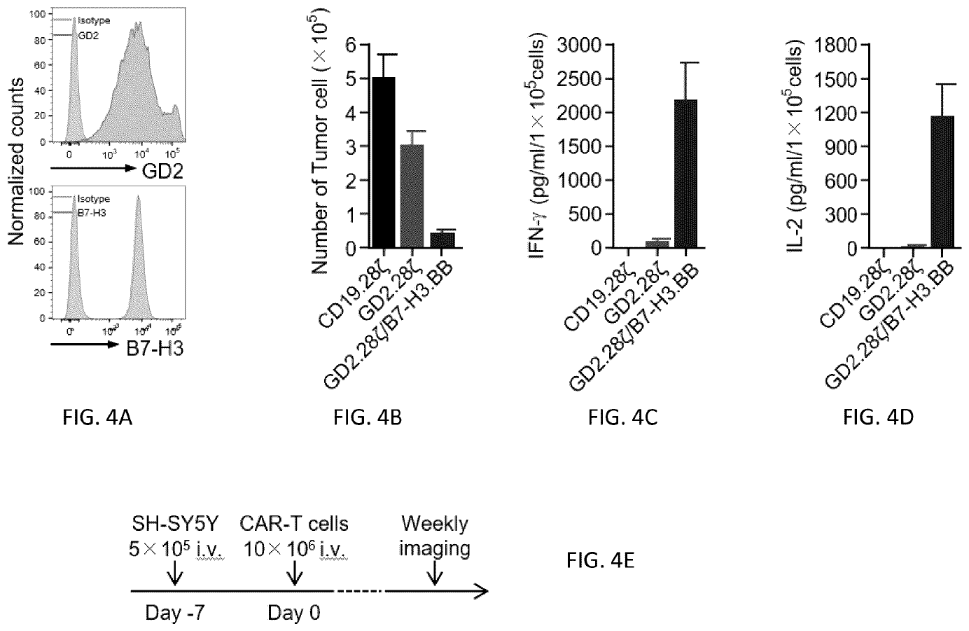

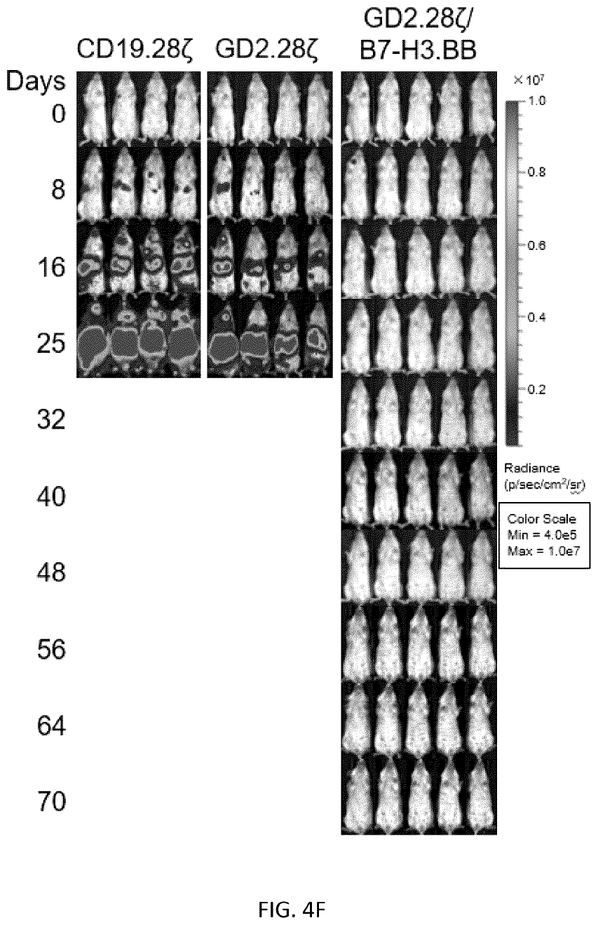

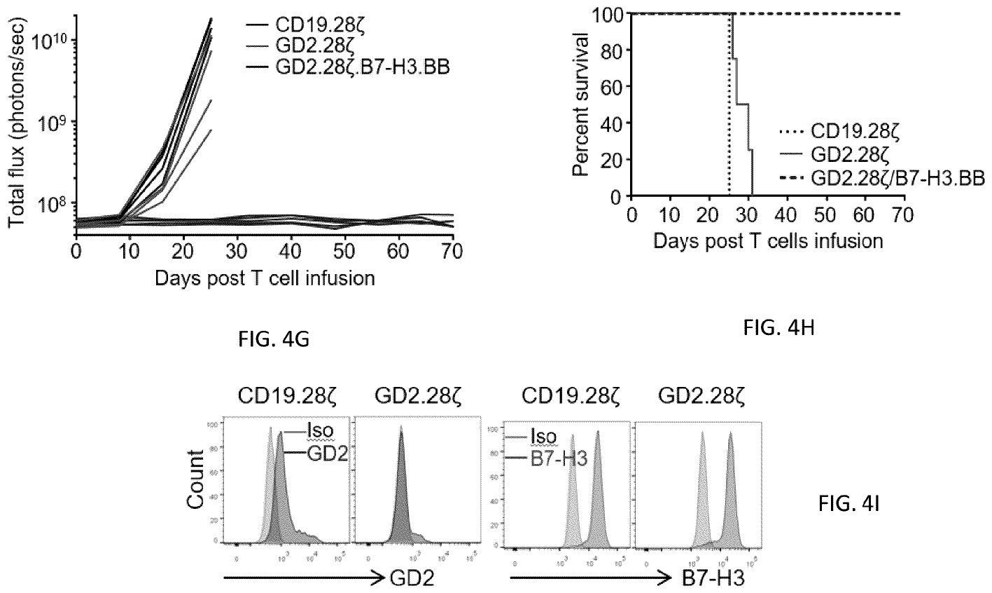

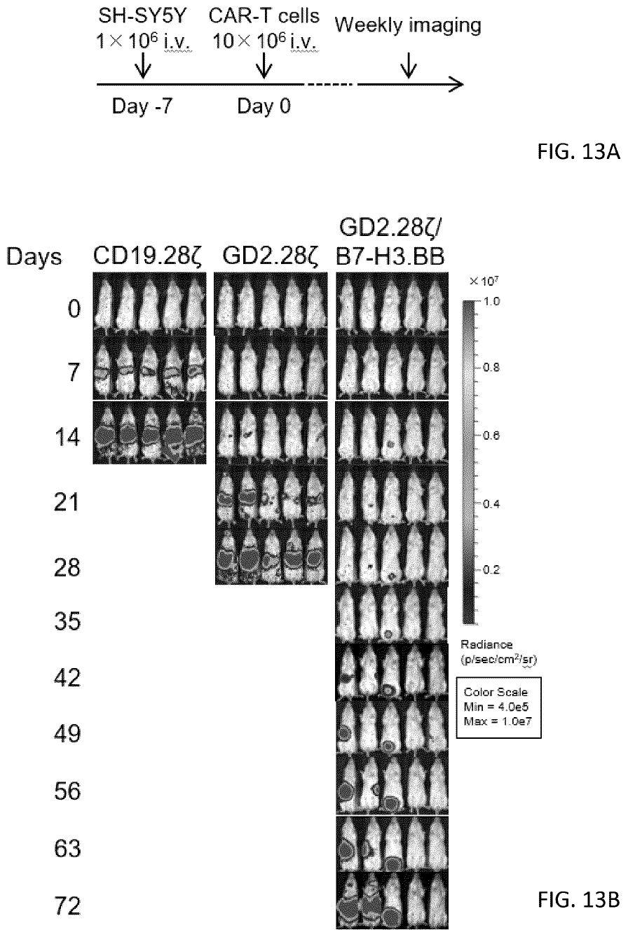

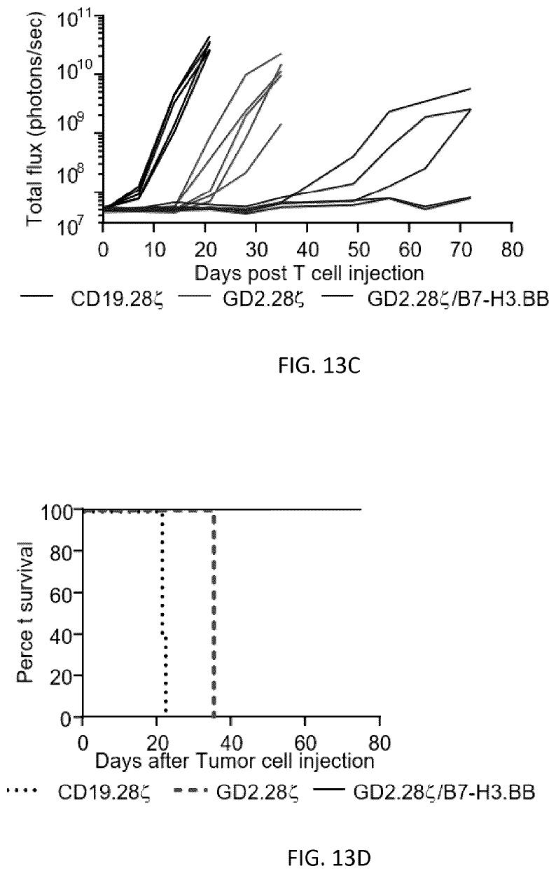

To evaluate if T cells expressing the GD2.28ζ.CAR/B7-H3.BB.CAR prevent tumor escape due to antigen loss the heterogeneous levels of GD2 expression in NB were leveraged on. T cells were co-cultured expressing either GD2.28ζ.CAR or GD2.28ζ.CAR/B7-H3.BB.CAR with the NB cell line SH-SY5Y that contains cells with dim expression of GD2 (FIG. 4A). T cells expressing the GD2.28ζ.CAR/B7-H3.BB.CAR exhibited the highest antitumor effects (FIG. 4B) and Th1 cytokine release (FIGS. 4C-4D). Next, T cells expressing the GD2.28ζ.CAR/B7-H3.BB.CAR were evaluated to determine if they can prevent tumor escape in vivo. In a low tumor burden model in SH-SY5Y-bearing NSG mice (FIG. 4E), T cells expressing the GD2.28ζ.CAR/B7-H3.BB.CAR fully controlled tumor growth (FIGS. 4F-4H). In mice treated with GD2.28ζ.CAR expressing T cells, growing tumors showed dim GD2 expression as compared to control mice treated with CD19-specific CAR-T cells, while showing unmodified expression of B7-H3 (FIG. 4I). Furthermore, T cells expressing the GD2.28ζ.CAR/B7-H3.BB.CAR effectively controlled the tumor growth in mice treated with higher tumor burden (FIGS. 13A-13C). Overall, these data indicate that T cells expressing dual targeting CARs with split co-stimulatory signal and one single CAR-CD3ζ domain can prevent tumor escape due to selection of tumor cells with dim antigen expression.

### DISCUSSION

Preventing tumor escape due to heterogeneity in antigen expression and providing optimal T cell co-stimulation remain critical aspects for achieving clinical responses with CAR-T cells in solid tumors. Here, CAR-T cells were generated that simultaneously target two antigens and provide optimal co-stimulation and T cell metabolic fitness by activating independently CD28 and 4-1BB pathways and tuning CD3ζ-chain-mediated signaling. In the clinically relevant model of NB, T cells expressing the dual CAR provided robust and sustained antitumor activity in in vivo stress conditions and prevented tumor escape due to heterogeneous antigen expression by tumor cells.

Targeting GD2 with CAR-T cells hold great clinical potential in NB^{15,17,24,30}. The approach has been proven safe, but the ideal co-stimulation of GD2-specific CAR-T cells in the clinical setting remains undefined. In particular, the most recent clinical study at Baylor College of Medicine in which a 3^{rd} generation CAR encoding both CD28 and OX40 endodomains demonstrated only modest clinical effects despite the combination with a checkpoint blockade¹⁵. Furthermore, while GD2 is widely recognized as an ideal target for immunotherapy of NB, its heterogeneous expression in tumor cells is not fully appreciated, and will lead to tumor recurrence due to the selection of tumor clones with low GD2 expression³¹⁻³⁴. A second clinically relevant NB target represented by B7-H3 was added¹⁹. Of note, GD2 and B7-H3 are physiologically expressed by NB, and the possibility to target these antigens simultaneously on tumor cells that naturally express the targets reflecting the physiologic density of antigen expression in tumor cells was tested.

The data demonstrates that it is possible to accommodate two almost complete CAR sequences targeting two different antigens into a single retroviral vector and obtain functional expression levels of both CARs in T cells. The first striking observation made is that T cells expressing two CARs, providing transacting CD28 and 4-1BB endodomains, and each one with its own CD3ζ chain did not show any beneficial effects in in vivo stress conditions. In sharp contrast, T cells expressing the same two CARs in which the CD3ζ chain is provided by one single CAR not only showed cytotoxic effects of each CAR, but also caused remarkable anti-tumour effects in vivo as compared to single targeting in stress conditions. The observation that a CAR lacking its own CD3ζ chain can still provide cytolytic effects to T cells if a second complete CAR is also expressed likely reflects unique characteristics of the synapse formed by the CAR molecules. While a CAR that lacks the CD3ζ cannot efficiently use the endogenous CD3ζ chains despite engaging the antigen, it can efficiently use the CD3ζ chain of a bystander CAR expressed by the same cell. Furthermore, this observation is in line with the attempts to tune the activity of CAR-T cells by modulating the level of CAR-CD3ζ-mediated signaling and indicate that two fully functional CAR-CD3ζ chains may promote an excessive signaling in the dual CAR format^{28,35}.

The second observation made is that CD28 and 4-1BB pathways transacting and independently activated via two distinct CARs is more effective than the classical in cis expression of 3^{rd} generation CARs. This is reminiscent of the proposed approach to supply 4-1BB ligand to CAR-T cells that encode CD28¹⁴. However, while 4-1BB ligand expression by CAR-T cells promotes the cross talk between T cells by engaging 4-1BB, the proposed approach has the significant advantage of inducting optimal co-stimulation of each single CAR-T cell independently when they encounter the tumor cells. Furthermore, the approach allows targeting two tumor-associated antigens preventing tumor escape.

The optimal stretch of co-stimulation provided by dual specific CARs with split co-stimulation and single CD3ζ chain is supported by signaling, molecular and metabolic properties. It has been demonstrated that sustained phosphorylation of the proximal signaling molecules of the CAR caused by the CD28 endodomain imprints CAR-T cells to promote rapid antitumor effects^{10,28,36}. In contrast, 4-1BB signaling in CAR-T cells provides the metabolic fitness characterized by the oxidative metabolism that is critical to sustain CAR-T cell persistence 29. CAR-T cells expressing the proposed CAR design are characterized by sustained CAR proximal signaling, molecular signature consistent with TCR tonic signal and metabolic profile providing rapid effector function via glycolysis, whilst preserving oxidative function for long-term persistence.

In summary, a strategy was designed that addresses simultaneously the most challenging tasks in solid tumors such as generating CAR-T cells that rapidly eliminate the tumor and functionally persist to control tumor growth upon tumor re-challenge. Furthermore, they prevent tumor relapse due to the emergence of tumor cells characterized by low antigen expression.

### Materials and Methods

### Cell Lines and Cell Culture

Human NB cell line IMR-32 was purchased from American Type Culture Collection. Human NB cell lines CHLA-255 and Firefly-luciferase (FFLuc)-CHLA-255 are gifts from Dr. Metelitsa at Baylor College of Medicine (Houston, TX), and LAN-1 is a gift from Dr. Brenner at Baylor College of Medicine (Houston, TX)²⁰,²¹ SH-SY5Y is gift from Dr. Chiarle at Boston Children's Hospital (Boston, MA)²². CHLA-255 and LAN-1 were cultured in RPMI-1640 (Gibco, Invitrogen) supplemented with 10% fetal bovine serum (Sigma), 2 mM GlutaMAX (Gibco), 100 unit/mL of penicillin (Gibco) and 100 μg/mL of streptomycin (Gibco). SH-SY5Y was cultured in DMEM (Gibco) supplemented with 10% fetal bovine serum (Sigma), 2 mM GlutaMAX (Gibco), 100 unit/mL of penicillin (Gibco) and 100 μg/mL of streptomycin (Gibco). Cells were maintained in a humidified atmosphere containing 5% C02 at 37° C. Tumor cell lines were transduced with a gamma retroviral vector encoding the enhanced green fluorescent protein (eGFP) and/or the FFLuc genes as described previously²³. All cell lines were mycoplasma free, and validated by flow cytometry for surface markers and functional readouts as needed.

### Plasmid Construction

The GD2-specific CARs encoding the modified GD2 specific single-chain variable fragment (14G2a scFv), the CD8α stalk and transmembrane domain, the CD28 or 4-1BB intracellular domain, and CD3ζ intracellular signaling domain was cloned into the retroviral vector SFG backbone. The modified 14G2a scFv was previously described²⁴. The B7-H3-specific CARs encoding the 376.96 scFv, the CD8α stalk and transmembrane domain, the CD28 or 4-1BB intracellular domain, and CD3ζ intracellular signaling domains was cloned into the retroviral vector SFG backbone¹⁹. The CD19-specific CARs encoding the FMC63 scFv, the CD8α stalk and transmembrane domain, the CD28 intracellular domain, and CD3ζ intracellular signaling domains was cloned into the retroviral vector SFG backbone²⁵. The vector cassette encoding the 14G2a scFv, the CD8α stalk and transmembrane domain, the CD28 intracellular domain, and CD3ζ intracellular signaling domain in combination with the 376.96 scFv, the CD8α stalk and transmembrane domain, and the 4-1BB intracellular signaling domain using a 2A-sequence peptide was generated by gene synthesis (GeneArt, Thermo Scientific) and was cloned into the retroviral vector SFG backbone.

Retrovirus preparation, transduction and expansion of human T cells Retroviral supernatants used for the transduction of human T cells were prepared as previously described²³. Briefly, 2×10⁶ 293T cells were seeded in 10 cm cell culture dish and transfected with the plasmid mixture of the retroviral transfer vector, the Peg-Pam-e plasmid encoding MoMLV gag-pol, and the RDF plasmid encoding the RD114 envelop, using GeneJuice transfection reagent (Navagen), according to the manufacturer's instruction. Supernatant containing the retrovirus was collected 48 and 72 hours after transfection, and filtered with 0.45 m filters. Buffy coats from healthy donors were purchased from the Gulf Coast Regional Blood Center, Houston, TX. Peripheral blood mononuclear cells (PBMCs) isolated with Lymphoprep density separation (Fresenius Kabi Norge) were activated on plates coated with 1 μg/mL CD3 (Miltenyi Biotec) and 1 μg/mL CD28 (BD Biosciences) agonistic monoclonal antibodies (mAbs). On day 2, T lymphocytes were transduced with retroviral supernatants using retronectin-coated plates (Takara Bio). Briefly, non-tissue culture treated 24-well plates are coated overnight with 7 μg/mL retronectin in the cold room, washed once 1 mL medium, coated with 1 mL of the retroviral supernatant per well and centrifuged at 2,000 g for 90 minutes. After removal of the supernatant, 5×10⁵ activated T cells were plated, and centrifuged at 1,000 g for 10 minutes. Three days later, T cells were collected and expanded in complete medium (45% RPMI-1640 and 45% Click's medium (Irvine Scientific), 10% Hyclone FBS (HyClone), 2 mM GlutaMAX (Gibco), 100 unit/mL of Penicillin (Gibco) and 100 μg/mL of streptomycin (Gibco) with IL-7 (10 ng/mL; PeproTech) and IL-15 (5 ng/mL; PeproTech), changing medium every 2-3 days. On day 12-14, cells were collected for in vitro and in vivo experiments. T cells were cultured in IL-7/IL-15 depleted medium for two days prior to being used in in vitro functional assays²⁶.

### Western Blot Analysis

Protein lysates were normalized according to the percentage of CAR expression and the number of T cell, and were resuspended in 2× Laemelli Buffer (Bio-Rad) in reducing condition (with β-mercaptoethanol). To assess signaling through the CAR, T cells on ice were incubated with 2 μg of the 1A7 anti-idiotype mAb specific for the 14G2a scFv and 1 μg of the 41g-B7-H3 fused with mouse Fc (Chimerigen Laboratories) for 20 minutes and then 2 μL of goat anti-mouse secondary Ab (BD Bioscience) for an additional 20 minutes. Cells were then transferred to a 37° C. water bath for the indicated time points and lysed with 2×Laemelli Buffer. All lysates were separated in 4-15% sodium dodecyl sulfate polyacrylamide gels (SDS-PAGE, Bio-Rad) and transferred to polyvinylidene diflouride membranes (Bio-Rad). After protein transfer onto polyvinylidene diflouride membranes, membranes were blocked in 5% non-fat milk in Tris-buffered saline, 0.1% Tween 20 (TBST) and incubated with primary at a dilution of 1 to 1,000 in TBST with 5% non-fat milk. The following abs were used: anti-CD3ζ (phospho Y142) (Abcam), anti-CD3ζ (Santa Cruz Biotechnology), anti-phospho-p44/42 MAPK (Thr202/Try204) (Cell Signaling), anti-ERK (pan) (BD Biosciences), anti-phospho-Akt (Ser473) (Cell Signaling), and anti-Akt (pan) (Cell Signaling). Membranes were then incubated with HRP-conjugated goat anti-mouse or goat anti-rabbit IgG (both Santa Cruz) at a dilution of 1 to 3,000 and developed with SuperSignal West Femto Maximum Sensitivity Substrate or SuperSignal West Pico Chemiluminescent Substrate (both Thermo Scientific) on the Gel station (Boi-Rad).

### Enzyme-Linked Immunosorbent Assay (ELISA)

Culture supernatants were collected after 24 hours of co-culture to measure the release of IFN-γ and IL-2 by using specific ELISA kit (R&D systems) following manufacturer's instructions. Each supernatant was measured in duplicate. Samples were measured using Synergy 2 Multi-Detection Microplate Reader and Gen5 software (both BioTek).

### Flow Cytometry

Flow cytometry was performed using Abs specific to human CD3, CD4, CD8, CD27, CD28, CD45, CD45RA, CCR7, PD-1, LAG-3, TIM-3, B7-H3 (clone 7-517), and GD2 (clone 14G2a) (all from BD Biosciences) conjugated with phycoerythrin (PE), PE-Cy7, fluorescein isothiocyanate (FITC), allophycocyanin (APC), APC-H7, Alexa Fluor 647 (AF 647), Brilliant Violet (BV) 421, BV510, BV605, and/or BV711. Expression of human B7-H3 in tumor cell lines was assessed with the 376.96 mAb and confirmed with another B7-H3 Ab clone 7-517 (BD Biosciences) (Imai et al., 1982). To detect CD19.CAR, an anti-idiotype mAb (clone 233-4A) specific for the CD19 scFv was used followed by either APC or PE conjugated rat anti-mouse IgG secondary mAb (BD Biosciences). To detect GD2.CAR, the 1A7 anti-idiotype mAb specific for the 14G2a scFv (Pule et al., 2005) was used followed by either APC or PE conjugated rat anti-mouse IgG secondary mAb (BD Biosciences). The expression of B7-H3.CAR was detected using the recombinant human B7-H3 Fc chimera protein (R&D systems) followed by the Alexa Fluor 647-conjugated AffiniPure F(ab′)2 Fragment Goat Anti-Human IgG (H+L) secondary mAb (Jackson ImmunoResearch). For absolute number calculations, samples were analyzed using CountBright absolute counting beads (Thermo Scientific). Samples were analyzed with BD FACScanto II or BD FACSfortessa (BD Biosciences) with the BD Diva software (BD Biosciences), for each sample a minimum of 10,000 events was acquired, and data was analyzed using Flowjo 10 (Tree Star). For the carboxyfluorescein diacetate succinimidyl ester (CSFE)-based proliferation assay, T cells were labeled with 1.5 mM CSFE (Invitrogen) and plated with tumor cells at the T cell to tumor cell ratio of 1 to 1. CFSE dilution was measured on gated T cells on day 5 using flow cytometry²⁷.

### Long-Term In Vitro Cytotoxicity and Repetitive Co-Culture Assay

Tumor cells were seeded in 24-well plates at a concentration of 5×10⁵ cells/well one day prior to the addition of T cells. Donor-matched T cells normalized for transduction efficiency were added at the T cell to tumor cell ratio of 1 to 5 without the addition of exogenous cytokines. On day 5 of co-culture, cells were collected and the frequency of T cells and residual tumor cells were measured by flow cytometry based on CD3 and GFP expression, respectively. Supernatant were also collected for cytokine measurements 24 hours after each cycle start. For each experiment, CD19.28ζ.CAR-Ts were used as negative control. Dead cells were gated out by Zombie Aqua Dye (Biolegend) staining while T cells were identified by the expression of CD3 and tumor cells by the expression of GFP.

For multiple rounds of co-culture, tumor cells were seeded at 5×10⁵ per well in 24-well plates one day prior to the addition of T cells. Donor-matched T cells normalized for transduction efficiency were added at the T cell to tumor cell ratio of 1 to 5. At day 4, 6, and 8, all T cells were harvested and transferred into a new well in which 5×10⁵ NB cells were seeded one day prior to the addition of T cells. T cells (CD3⁺) and NB cells (GFP+) were quantified by flow cytometry with CountBright absolute counting beads (Thermo Scientific) after 4 cycles (day 4, 6, 8, and 12) based on CD3 and GFP expression, respectively. Supernatant were also collected for cytokine measurements 24 hours after each cycle start. For each experiment, CD19.28ζ.CAR-T cells were used as negative control. Dead cells were gated out by Zombie Aqua Dye (Biolegend) staining while T cells were identified by the expression of CD3 and tumor cells by the expression of GFP.

### Antigen Density Assay

Quantification of GD2 and B7-H3 on the NB cells was performed using the QIFIKIT (Dako, Denmark) following manufacturer's instructions. Briefly, measurements of cell-bound mAb were made by quantitative flow cytometry using mAb-coated beads calibrated from background level to 787,000 molecules used as internal standards in the indirect analysis. NB cells were incubated for 30 min at 4° C. with/without either 5 μg of GD2 Ab (clone 14G2a) or B7-H3 Ab (clone 376.96). Following two washes in DPBS/2 mM EDTA/2% FBS, NB cells were incubated for 30 min at 4° C. with a 1 to 50 dilution of F(ab′)2, Fragment of FITC-Conjugated Goat-Anti-Mouse Immunoglobulins. Following one wash in DPBS (Corning)/2 mM EDTA (Thermo Scientific)/2% FBS (HyClone), the mAb-calibrated beads (100 μL/tube) were incubated for 30 min at 4° C. with a 1 to 50 dilution of F(ab′)2, Fragment of FITC-Conjugated Goat-Anti-Mouse Immunoglobulins. After two washes, NB cells and standard beads were resuspended in 300 μL of DPBS/2 mM EDTA/2% FBS and analyzed by flow cytometry. Beads were used to construct a calibration curve obtained by plotting the fluorescence mean intensity versus the number of mAb molecules after correction for background mean fluorescence. The equation of the linear regression curve was used to calculate the mean number of mAb molecules bound per cell from the mean fluorescence intensity.

### Reverse Transcription Quantitative Polymerase Chain Reaction

Cells were lysed and RNA was extracted using the RNeasy Minikit (Qiagen) and reverse transcribed into cDNA (Superscript VILO, Invitrogen). Human IFN-7, CXCL10, IFIT1, IRF7, TCF7, DUSP4, KLRG1, CD300A, and GZMB mRNA expression was quantified using TaqMan probes (Thermo Scientific) on a QuantStudio 6 PCR machine (Applied Biosystems) using 18S ribosomal RNA as housekeeping gene control (Invitrogen).

### RNA-Sequence Analysis

Briefly, total RNA was extracted from CAR-T cells, and mRNA libraries were prepared (TruSeq Stranded mRNA Library Prep, Illumina) and sequenced on the Illumina HiSeq4000 platform (UNC High-Throughput Sequencing Facility) using paired-end 100-bp reads, with 84 million reads on average (range, 49-139 million). RNA-seq data were aligned with STAR alignment (v2.4.2) and quantified with Salmon (v0.6.0). Differential gene-expression analysis was performed using the R DESEq2 package (https://genomebiology.biomedcentral.com/articles/10.1186/s13059-014-0550-8). Among all significantly expressed genes between GD2.28ζ-CAR-T cells and GD2.28ζ plus B7-H3.BB.CAR-T cells (FDRp <0.05), expression was further filtered to genes contained within the IFN-γ and the pathway signatures.

Activation of CAR-T Cells for RNA-Seq, qPCR, and Cell Metabolic Assay

One mg/mL of the 1A7 anti-idiotype mAb specific for the 14G2a scFv and 1 g/ml of the 41g-B7-H3 fused with mouse Fc (Chimerigen Laboratories) were coated on non-tissue culture treated 24-well plates for 16 hours in the cold room. Plates were washed with DPBS (Corning) three times before plating T cells (1×10⁶ cells/well). CAR-T cells were incubated on 1A7 mAb and 4Ig-B7-H3-coated plates at 37° C. for 24 hours. Then, CAR-T cells were transferred to 24-well tissue culture plate. T cell metabolism was measured in a Seahorse XFe24 analyzer (Seahorse Bioscience). 5×10⁵ T cells were seeded to 24 well Seahorse XF-24 assay plates in Seahorse BASE media with additives. Cells were incubated at 37° C. in a non-C02 incubator for 45 min. All media was adjusted to pH 7.4 on the day of assay. Mitochondrial and glycolysis stress tests were performed according to the manufacturer's protocol. Oxygen consumption and extracellular acidification rates were automatically calculated and recorded by the Seahorse XF-24 software.

### Xenograft NB Mouse Models

Mouse experiments were performed in accordance with the University of North Carolina (UNC) animal husbandry guidelines according to protocols approved by the UNC institutional animal care and use committee. For these studies, 7-10 weeks old female NSG mice (NOD.Cg-Prkdc^{scid} IL2rg^{tm1Wjl}/SzJ, UNC Animal Studies Core Facility) received 2×10⁶ of FFLuc-CHLA-255, 4×10⁶ of FFLuc-LAN-1, 0.5×10⁶ or 1×10⁶ of FFLuc-SH-SY5Y intravenously via tail vein. Tumor growth was monitored by bioluminescence imaging using the IVIS lumina II in vivo imaging system (PerkinElmer). For the study, tumor cells suspended in 200 μL DPBS (Coming) were injected intravenously via tail vein using 28-gauge needle. Fourteen to 21 days after tumor cell injection, 2 or 6×10⁶ CAR-positive T cells were injected intravenously via tail vein. To inject same T cell number for each mouse, total T cell number was adjusted to the largest number by adding non-transduced T cells. Mice were bled at specific intervals (10-15 days, as per UNC-IACUC guidelines) to measure T cell frequency and/or phenotype. Mice were euthanized when signs of discomfort were detected by the investigators or as recommended by the veterinarian who monitored the mice three times a week. At the time of euthanasia, blood, spleen, and/or bone marrow were isolated and analyzed to detect CAR-T cells. In tumor re-challenge experiments, tumor-bearing mice were further injected intravenously via tail vein with FFLuc-CHLA-255 cells, FFLuc-LAN-1, or FFLuc-SH-SY5Y.

### Statistical Analyses

Data are reported as the mean and the unpaired and nonparametric Mann Whitney test with two tailed p value calculation was used to measure differences between two groups. For multiple group comparisons, one-way analysis of variance (ANOVA) or two-way ANOVA was used to determine statistically significant differences between samples. Tukey's multiple comparison test adjusted p value <0.05 indicates a significant difference. Measurements were summarized as mean±SEM as noted in the figure legends. Differences between the bioluminescence of tumor and the survival curves were analyzed by the Chi-square test using GraphPad Prism v8. Experimental sample numbers (n) are indicated in the figure legends. The statistical analysis method is also described in the individual figure legends. Graph generation and statistical analyses were performed using the GraphPad Prism software (GraphPad Software).

### Example 2

This Example both re-presents certain data from Example 1 and provides additional data.

### Results

### Single or Dual Targeting do not Eradicate Tumor in Stress Conditions

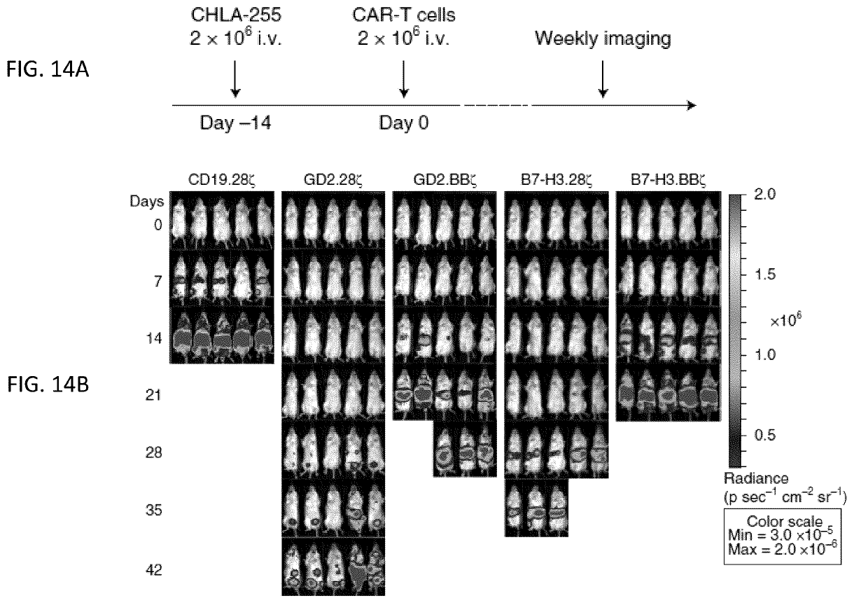

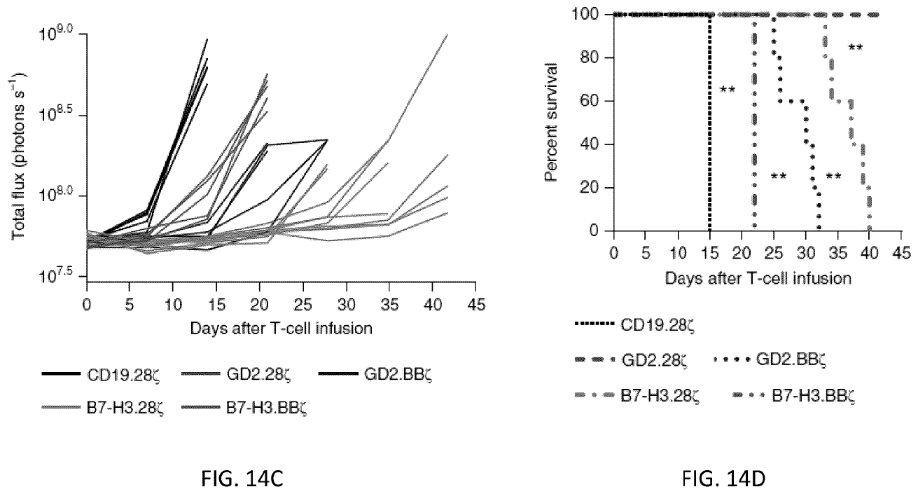

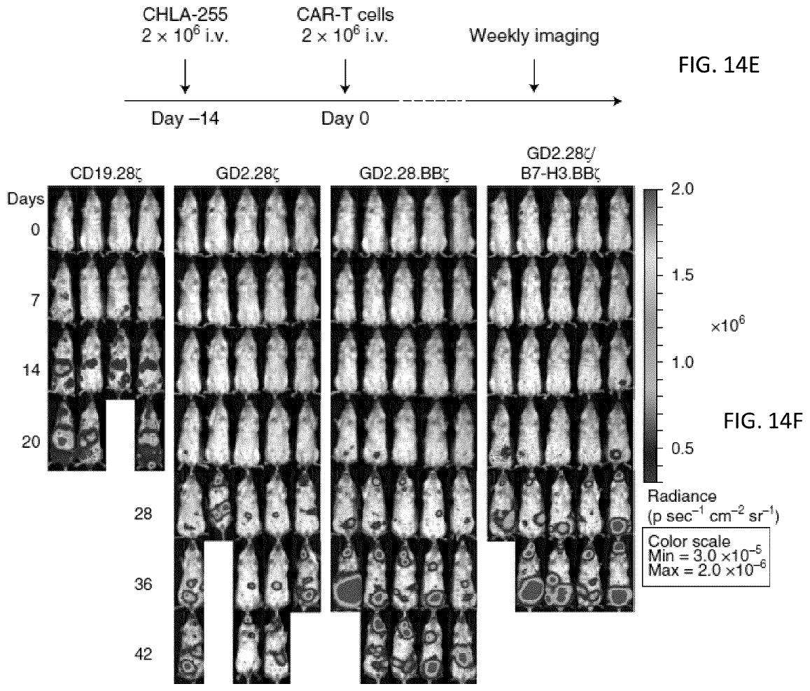

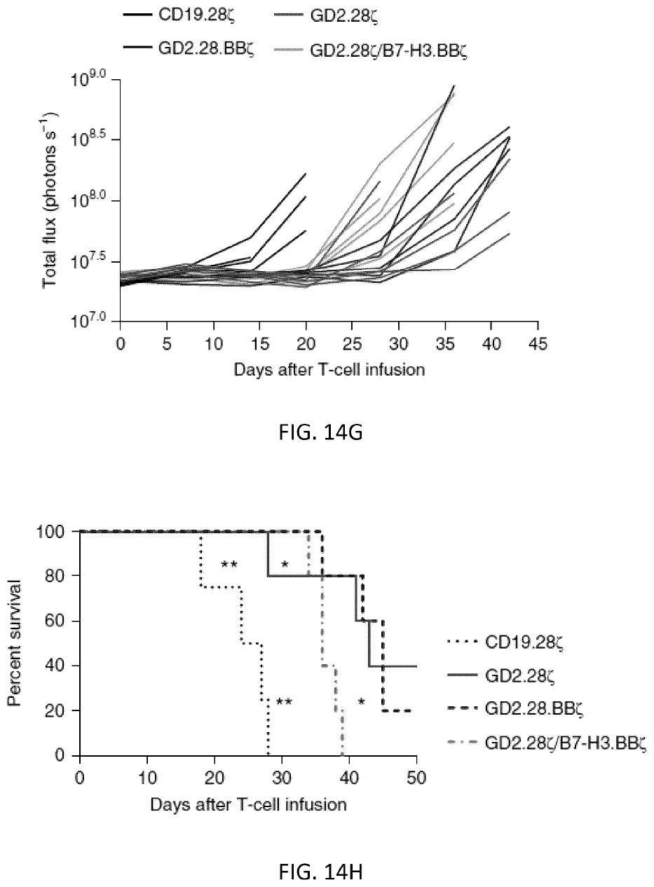

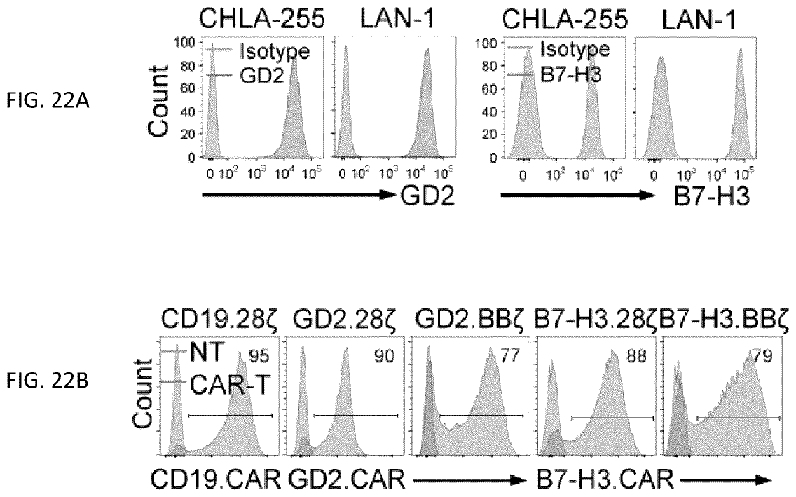

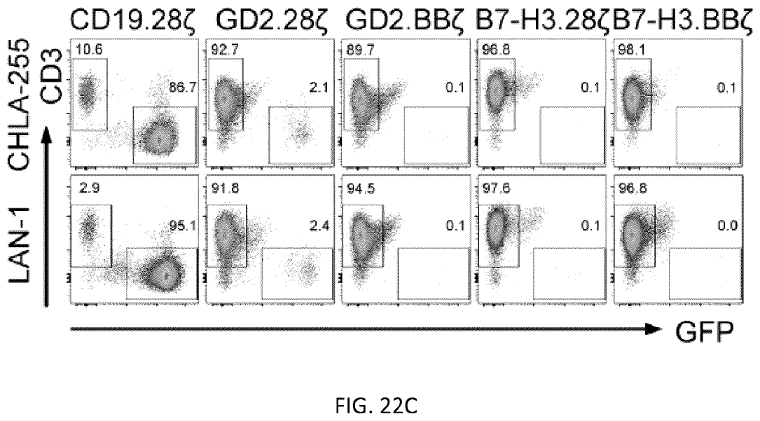

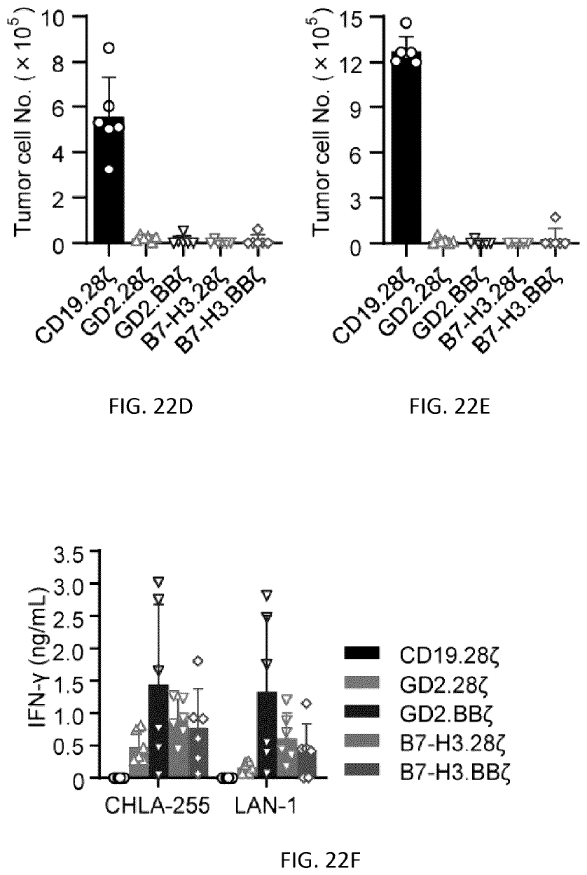

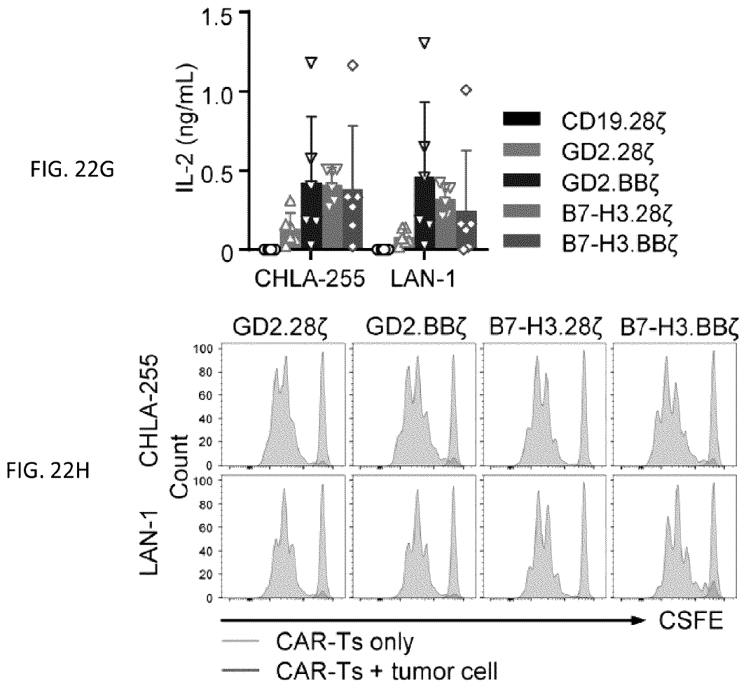

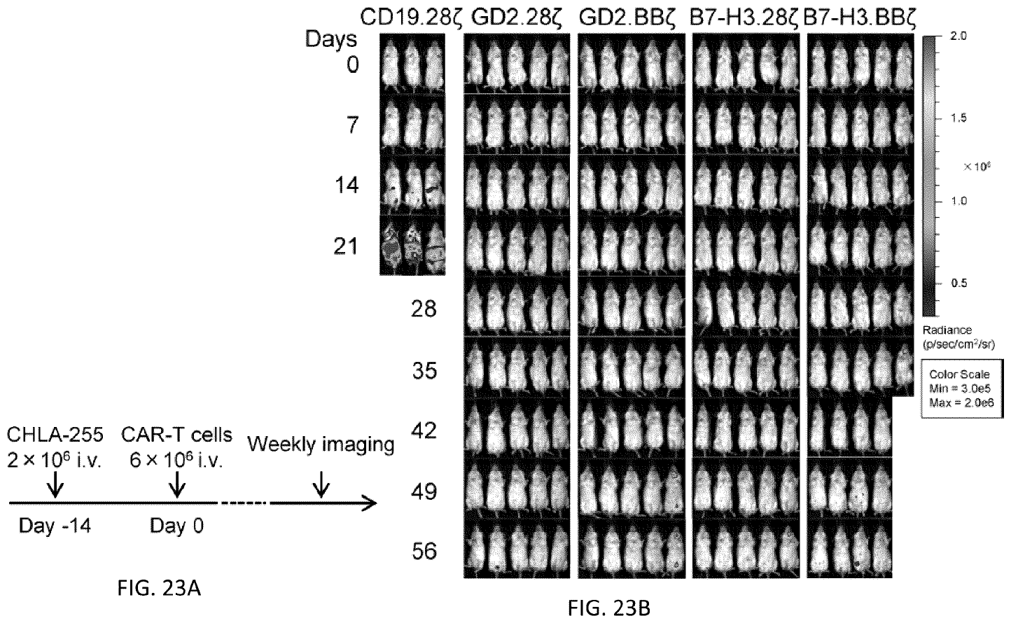

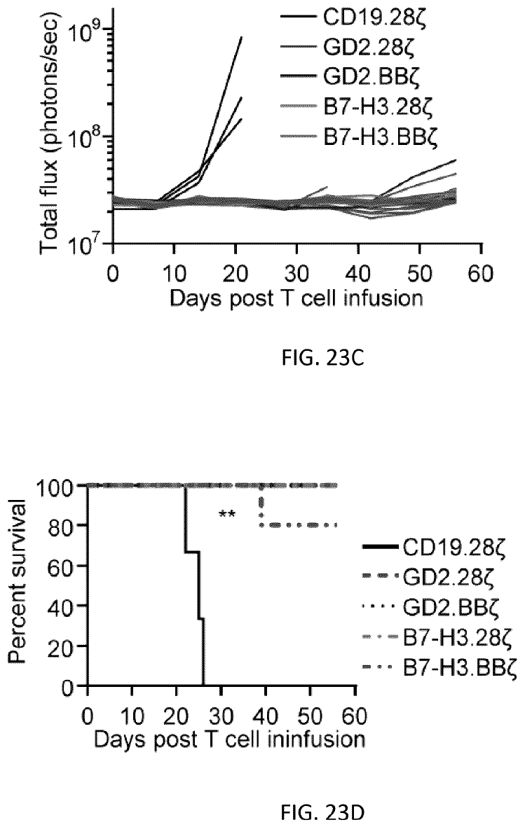

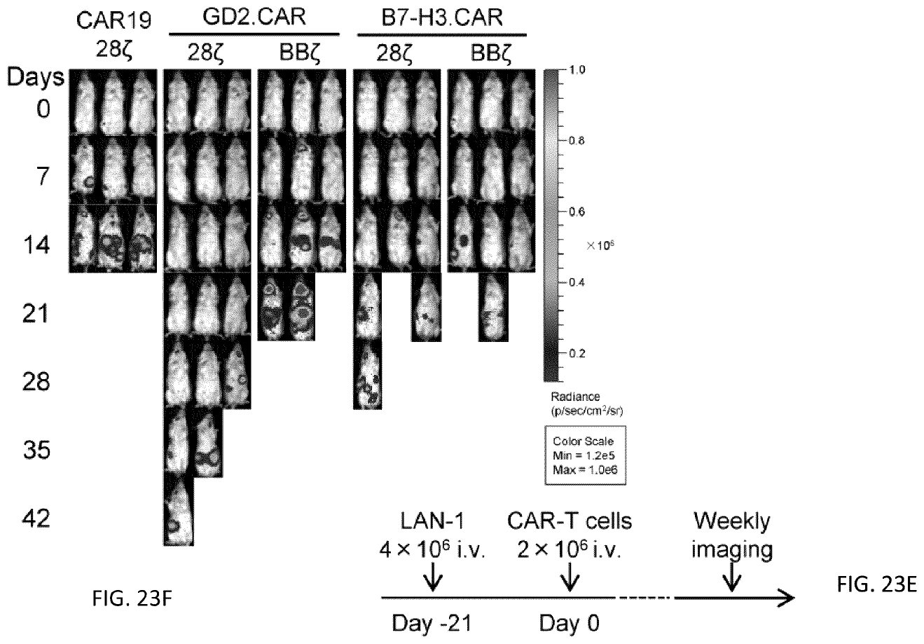

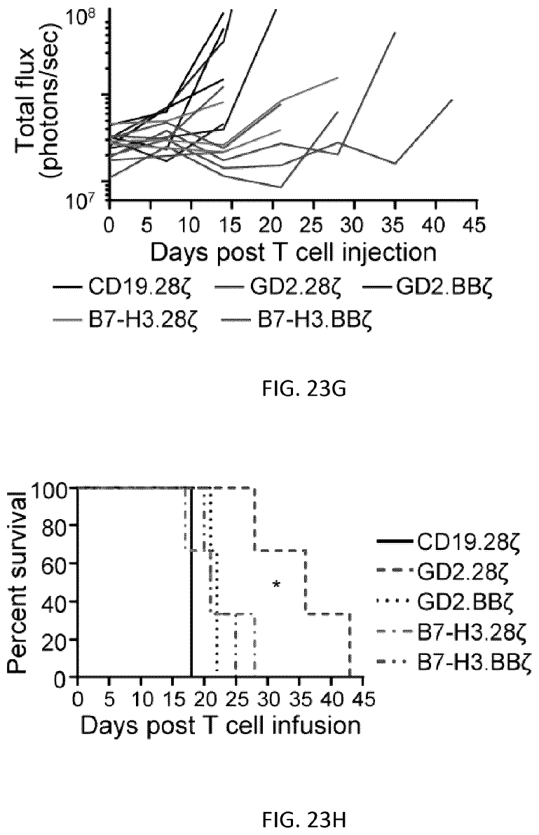

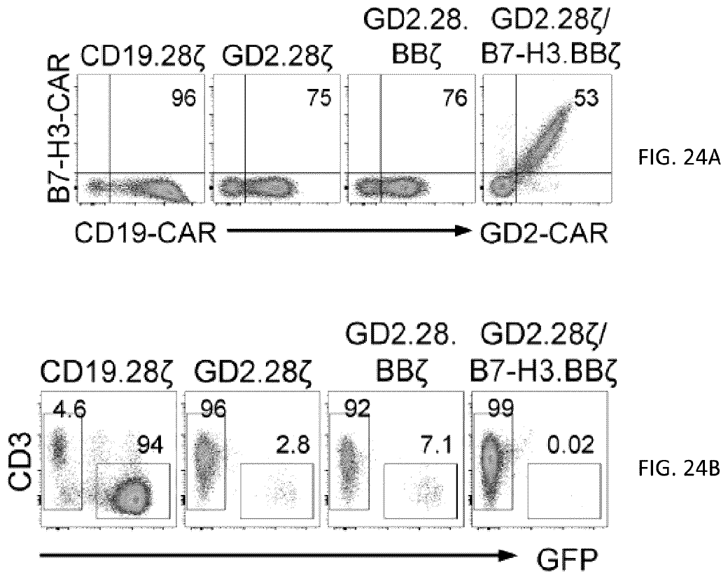

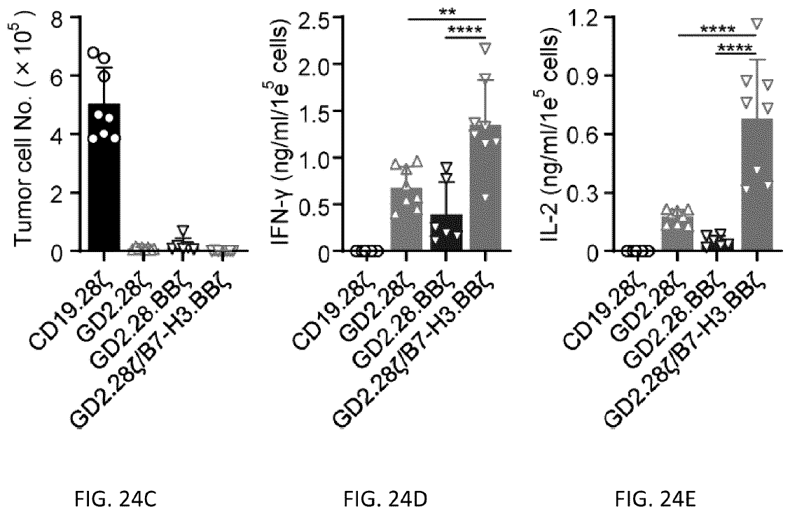

NB was used as a tumor model, and specifically two tumor cell lines (CHLA-255 and LAN-1) that co-express two targetable antigens, GD2 and B7-H3, as assessed by flow cytometry (FIG. 22A). T cells engineered to express the GD2.28ζ, GD2.BBζ, B7-H3.28ζ, and B7-H3.BBζ CARs effectively eliminated tumor cells in vitro without any significant differences (FIGS. 22C-22E). The cytolytic activity of CAR-T cells was corroborated by IFN-γ and IL-2 release in the culture supernatant (FIGS. 22F-22G), and by T cell proliferation in response to NB cell lines (FIG. 22H). GD2.28ζ, GD2.BBζ, B7-H3.28ζ and B7-H3.BBζ CAR-T cells controlled tumor growth in NSG mice engrafted with CHLA-255 when high dose of CAR-T cells (6×10⁶ CAR-T cells) was used (FIGS. 23A-23D). In sharp contrast, when CAR-T cells were used in stress conditions such as those in which CHLA-255-bearing mice are treated with 2×10⁶ CAR-T cells (FIG. 14A), GD2.28ζ CAR-T cells exhibited superior tumor control as compared to GD2.BBζ, B7-H3.28ζ, and B7-H3.BBζ CAR-T cells (FIGS. 14B-14D). Similar results were observed in a second NB model in which mice were engrafted with LAN-1 cells (FIGS. 23E-23H). Since GD2.28ζ CAR-T cells showed the most prominent antitumor activity, but do not fully eradicate the tumor at low doses, the addition of 4-1BB costimulation was tested to see if it may lead to tumor eradication as previously described¹¹. 4-1BB costimulation was provided in the form of a 3^{rd} generation CAR (GD2.28.BBζ). A vector was constructed encoding simultaneously the GD2.28ζ and B7-H3.BBζ CARs (GD2.28ζ/B7-H3.BBζ) to provide both dual specificity and dual costimulation using two independent CAR molecules. Both in vitro (FIG. 24) and in vivo (FIGS. 14E-14H) experiments showed that GD2.28.BBζ and GD2.28ζ/B7-H3.BBζ CAR-T cells did not show superior antitumor activity compared to GD2.28ζ CAR-T cells in stress conditions. Overall, these data demonstrate that GD2.28ζ CAR-T cells are the most effective in controlling the tumor growth at low doses, but do not eradicate the tumor. Furthermore, 4-1BB costimulation provided either in the form of 3^{rd} generation CAR or dual CARs does not enhance the therapeutic effect.

### One Shared CD3ζ Triggers Dual CAR-T Cells

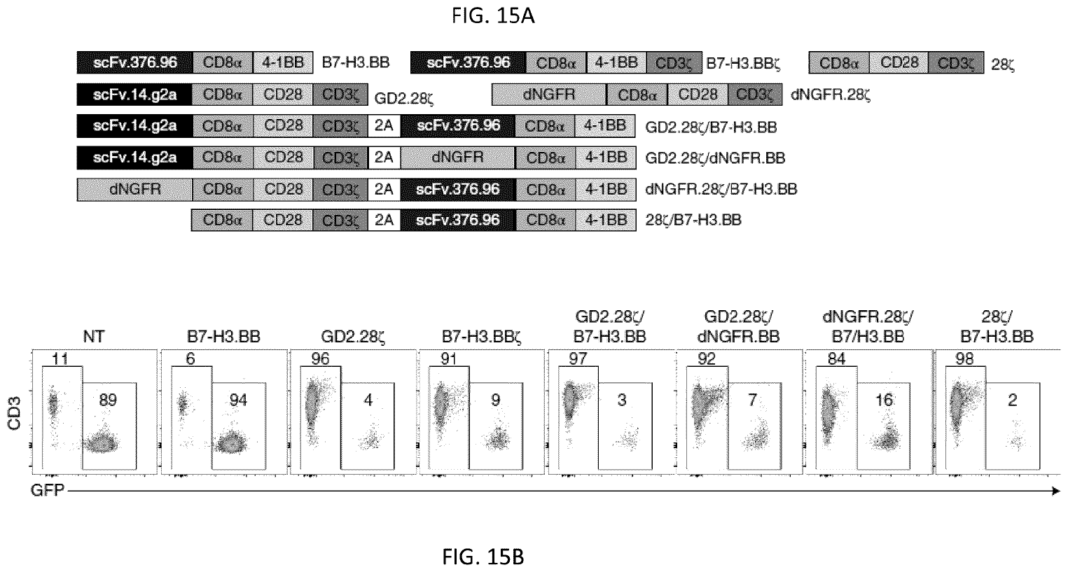

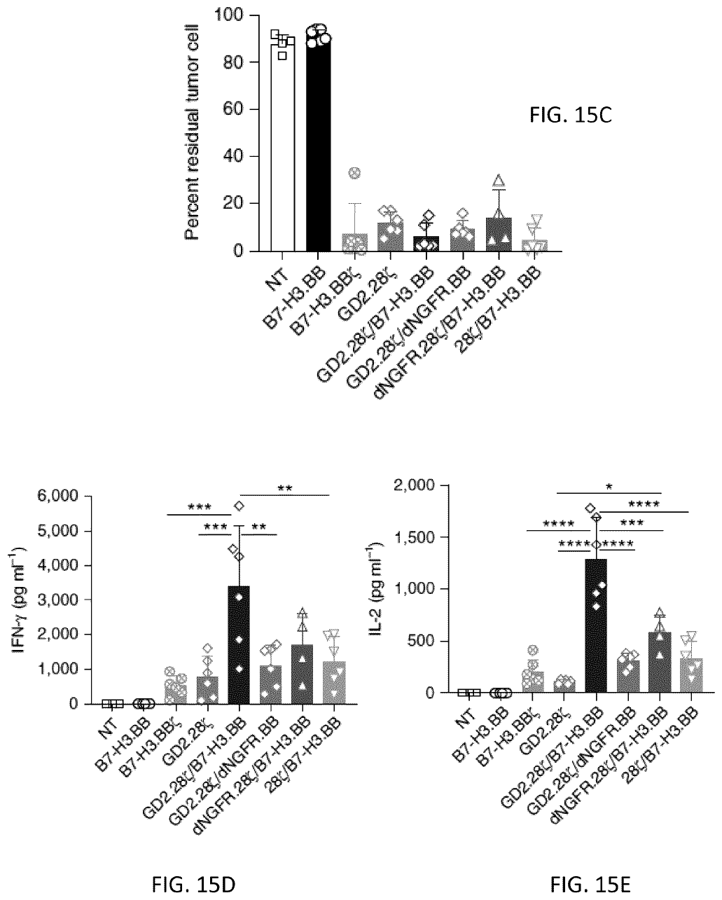

It was hypothesized that GD2.28ζ/B7-H3.BBζ CAR-T cells may receive excessive CD3ζ signaling that compromises the beneficial effects of the dual targeting and dual costimulation. A series of dual CARs encoded in a single retroviral vector were generated to assess if a shared CD3ζ chain is sufficient to provide optimal activation signaling for dual antigen targeting, and the role of each single antigen recognition and costimulation in the dual target format (FIG. 15A). It was found that GD2.28ζ, GD2.28ζ/B7-H3.BB and GD2.28ζ/dNGFR.BB CAR-Ts recognized the tumor cells as expected because they express a fully functional GD2.CAR. Similarly, B7-H3.BBζ CAR-T cells recognized tumor cells because they express a fully functional B7-H3.CAR, while B7-H3.BB CAR-T cells did not recognize the tumor because the CAR lacks CD3ζ (FIGS. 15B-15E). Remarkably, it was found that B7-H3.BB CAR-T cells acquired cytolytic activity and released cytokines towards B7-H3+tumor cells when they coexpressed either dNGFR.28ζ or 28ζ (FIGS. 15B-15E). This indicates that the incomplete B7-H3.BB CAR engaging the antigen can use the CD3ζ expressed in cis provided by another moiety that is not directly recognizing the antigen. Furthermore, when the B7-H3.BB CAR is coexpressed with the GD2.28ζ CAR, and when the two CARs are simultaneously engaging their antigens, CAR-T cells released significantly higher level of cytokines compared to GD2.28ζ CAR-T cells indicating that additional co-stimulatory effect is provided by 4-1BB (FIGS. 15D-15E). In T cells co-expressing the GD2.28ζ CAR and dNGFR.BBζ, which prevents the engagement of B7-H3, the additional effect of 4-1BB in promoting higher cytokine release was abolished, indicating that the costimulatory effect from 4-1BB is only mediated when both CARs engage their target antigen (FIGS. 15D-15E).

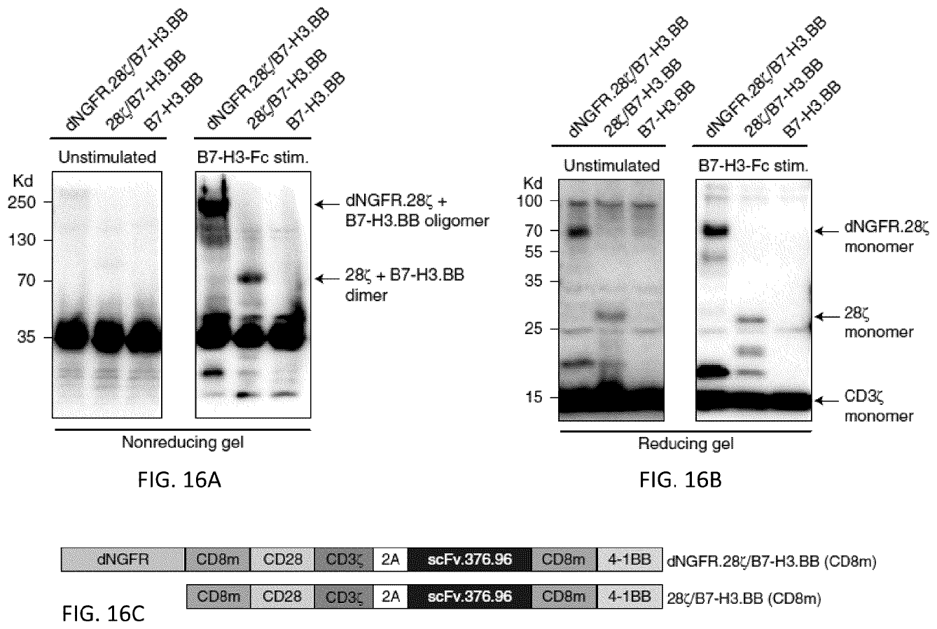

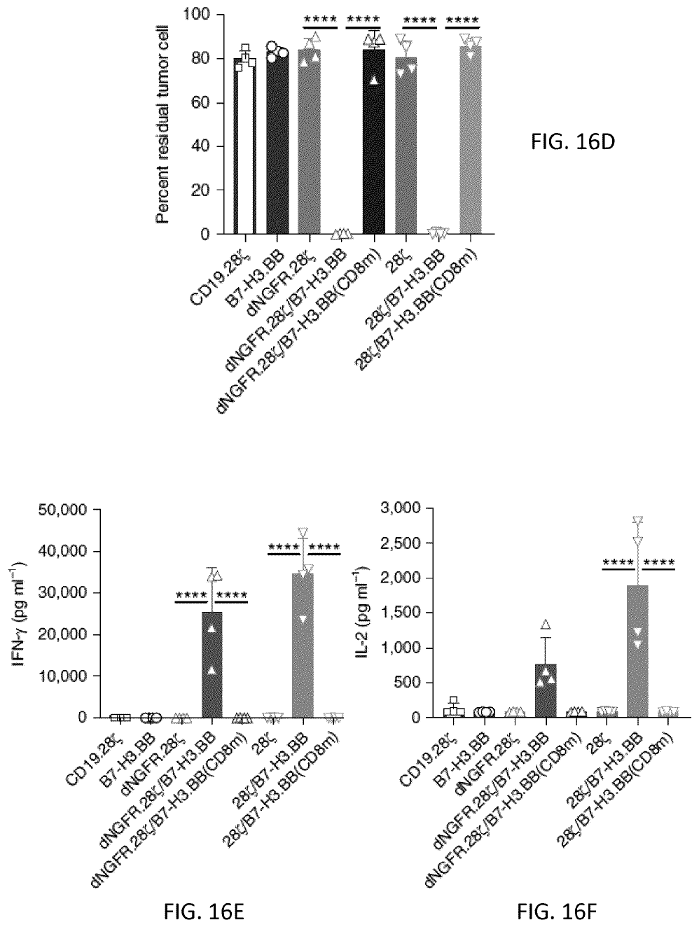

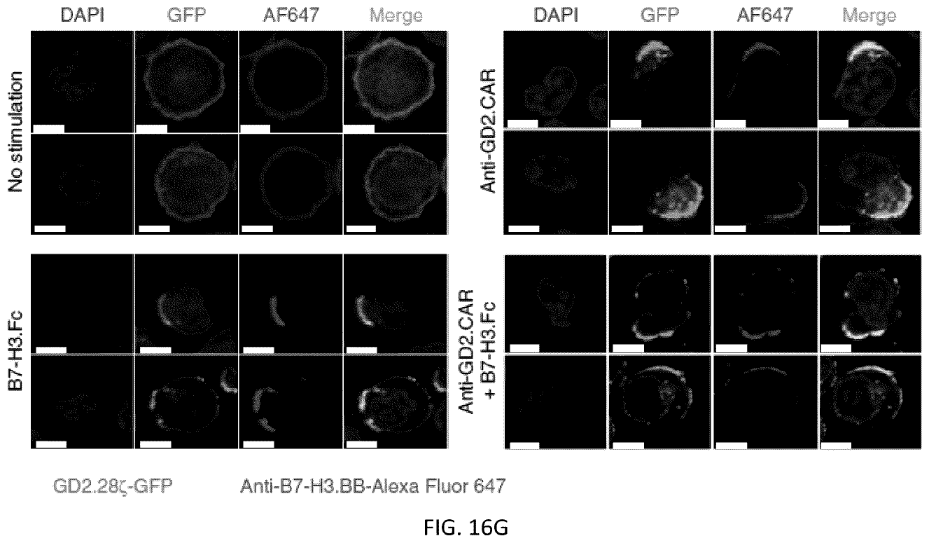

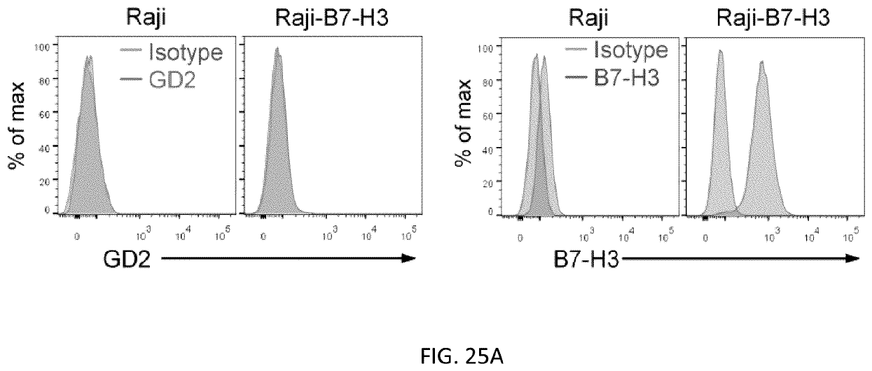

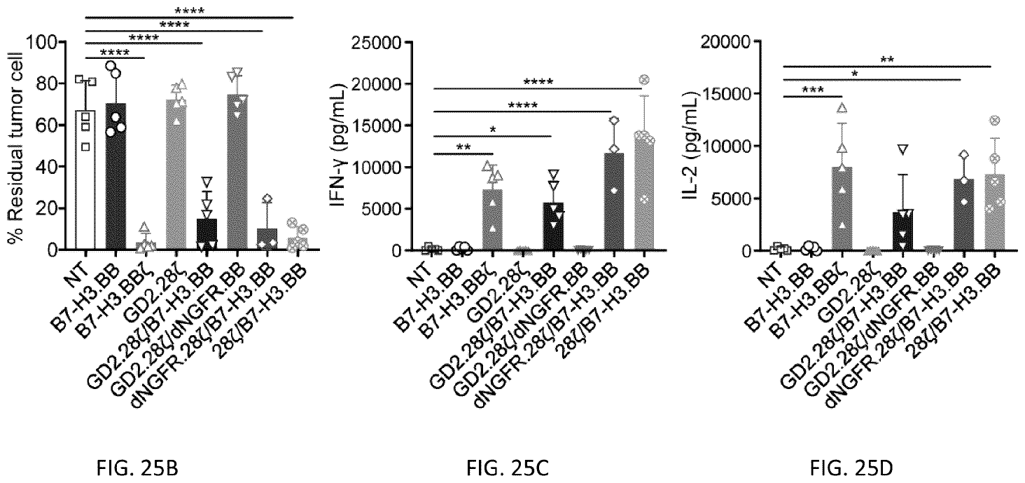

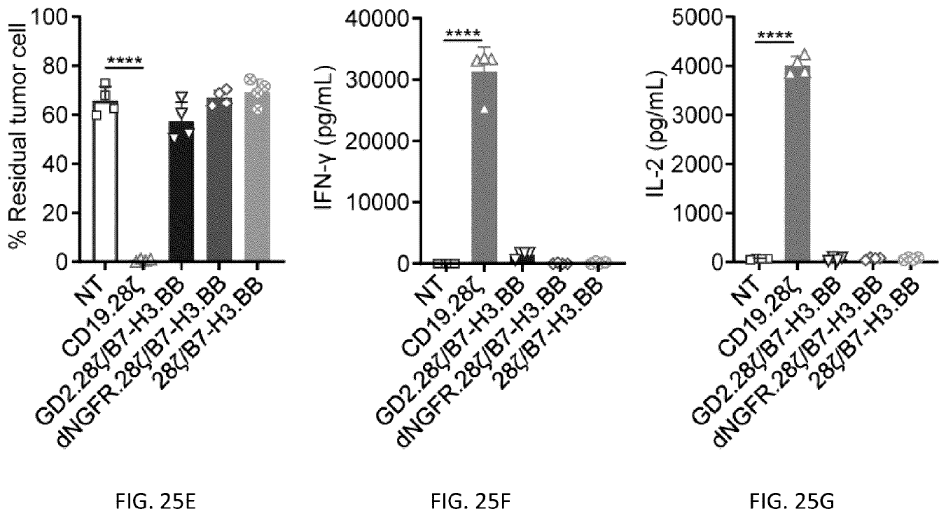

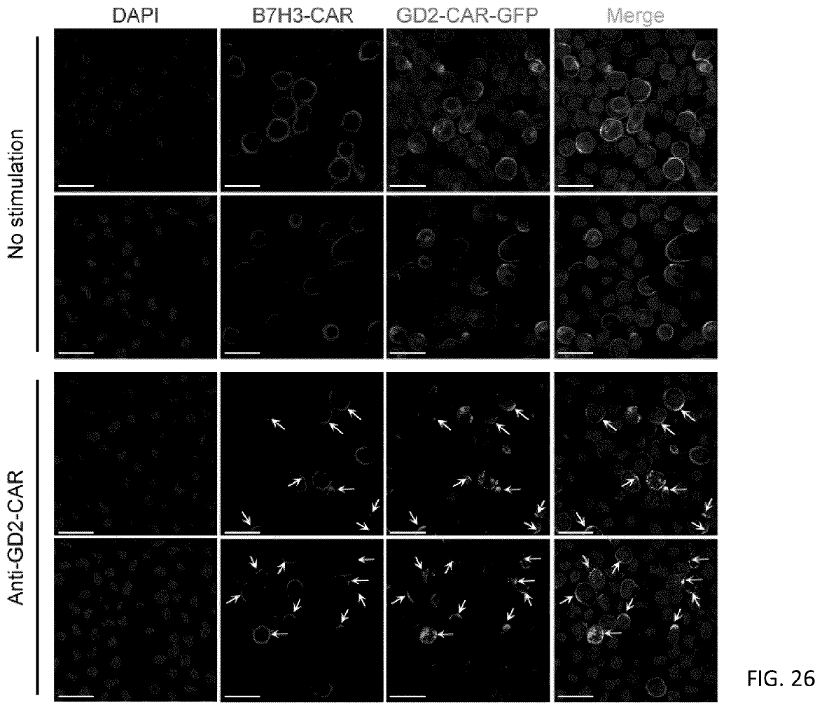

To investigate if two CAR molecules sharing one single CD3ζ form heterodimers after engaging their cognate antigens, reducing and non-reducing gel separation and western blots were performed. It was found that in B7-H3.BB CAR-T cells co-expressing either dNGFR.28ζ or 28ζ molecules and stimulated with the B7-H3-Fc protein followed by cross linking with a secondary anti-Fc antibody, the B7-H3.BB CAR formed heterodimers or oligomers with dNGFR.28ζ or 28ζ molecules, while dimers or oligomers were not observed without antigen stimulation (FIGS. 16A-16B). Since the two cysteine residues located at the positions 164 and 181 in the CD8α stalk region are involved in the dimerization of CAR molecules²⁵, C164S and C181S mutations in the CD8 stalk (CD8m) were generated and if these two cysteine residues are critical in mediating the dimerization of the B7-H3.BB CAR with the dNGFR.28ζ or 28ζ molecules was investigated. CAR-T cells engineered with the constructs carrying the mutated cysteine residues did not show cytotoxic activity and cytokine release when co-cultured with the CHLA-255 cell line (FIGS. 16C-16F). To further investigate the role of antigen engagement in mediating the dimerization of the CAR molecules, co-culture experiments were performed using Raji cells genetically modified to express B7-H3, but lacking GD2 expression (FIG. 25A). T cells co-expressing B7-H3.BB CAR and GD2.28ζ CAR, dNGFR.28ζ or 28ζ efficiently targeted B7-H3-expressing Raji cells, and released IFN-γ and IL-2, while T cells co-expressing the GD2.28ζ CAR and dNGFR.BB did not show antitumor activity (FIGS. 25B-25D). Control co-cultures with wild type Raji cells, also did not show antitumor activity (FIGS. 25E-25G) indicating that the cytolytic activity of dual CAR-T cells remains antigen depended.

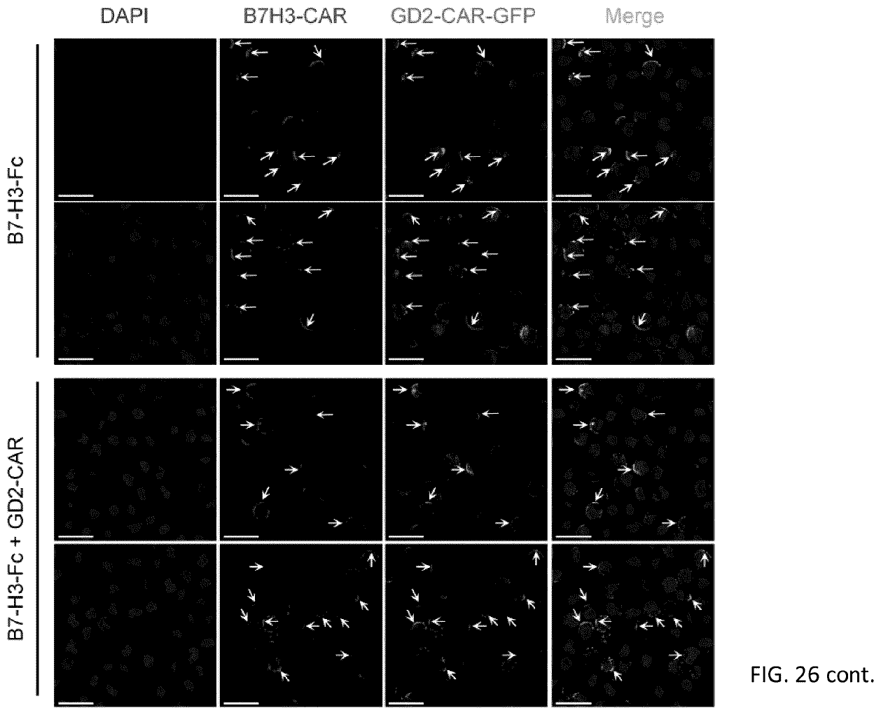

To investigate if the two CARs in GD2.28ζ/B7-H3.BB CAR-T cells can cluster in the same immune synapse upon engaging with either one of the antigens and then trigger activation signal through the shared CD3ζ chain, GFP was fused with GD2.28ζ and co-transduced T cell with B7-H3.BBζ, and then stimulated them with either the anti-GD2.CAR idiotype antibody 1A7 or the B7-H3-Fc fusion protein, or both. Using confocal microscopy imaging, it was found that GD2.28ζ and B7-H3.BB CARs formed membrane clusters and co-localized with each other upon CAR crosslinking via single or dual CAR engagement (FIG. 16G and FIG. 26).

### Dual CAR-T Cells Show Improved Functions

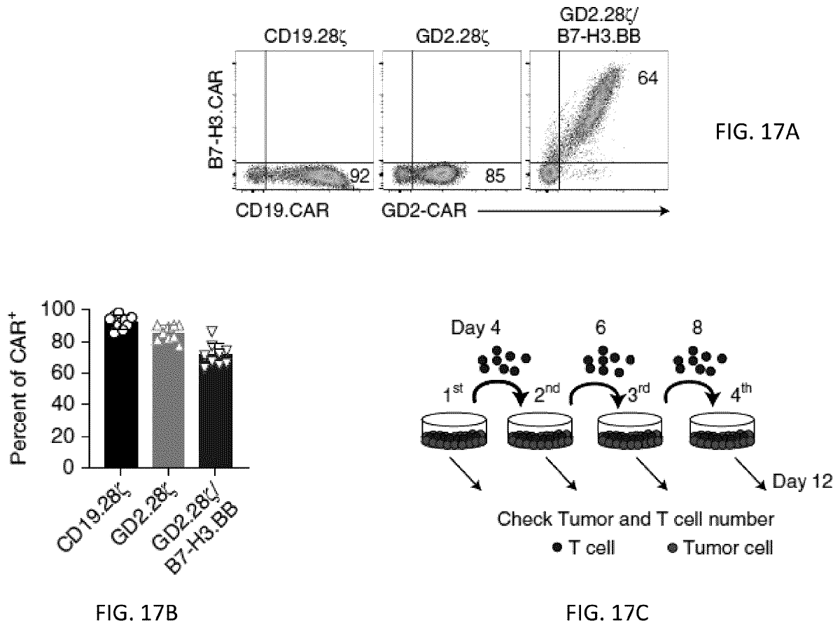

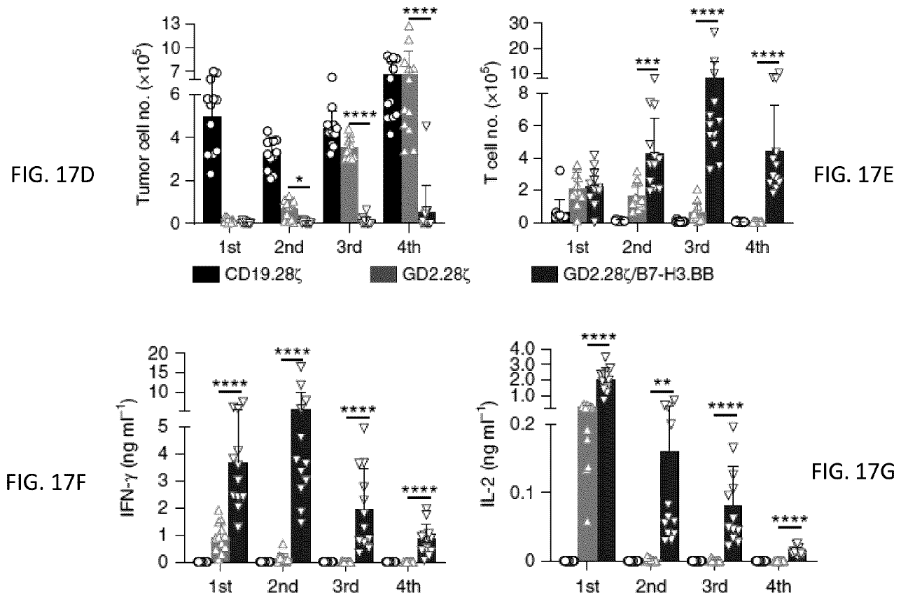

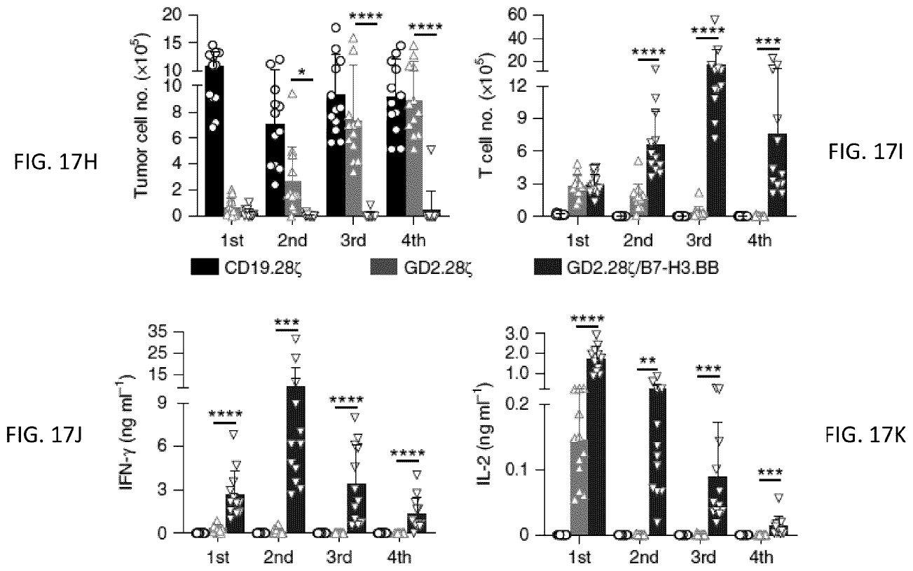

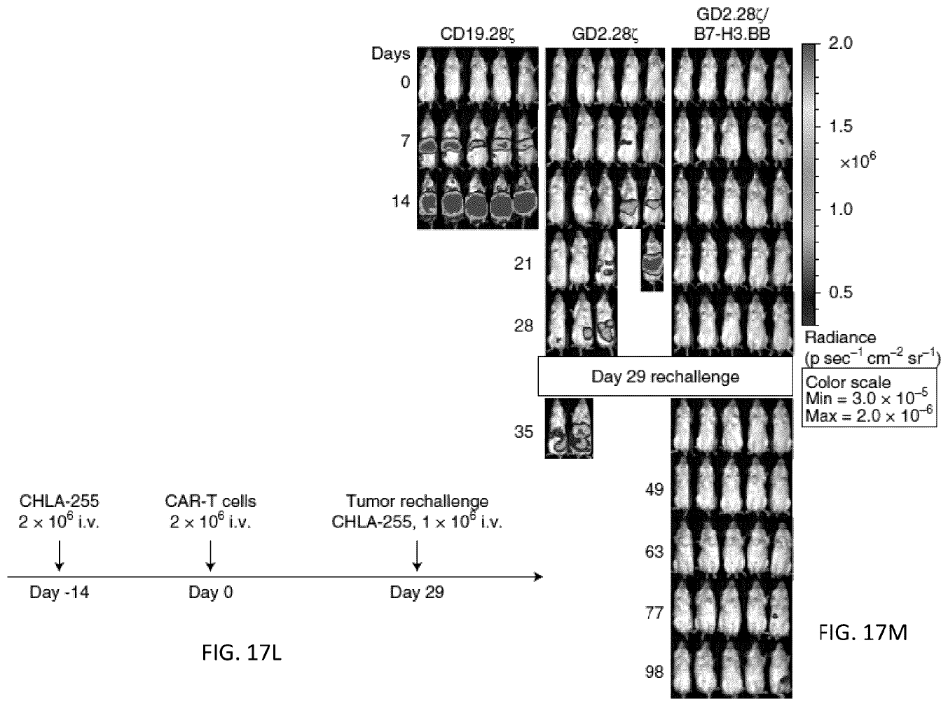

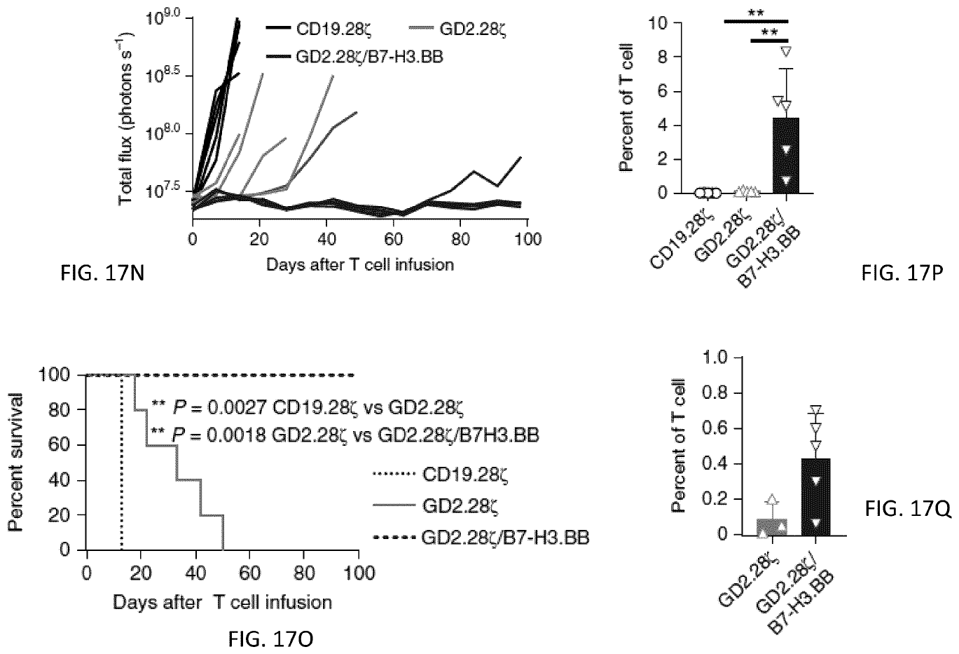

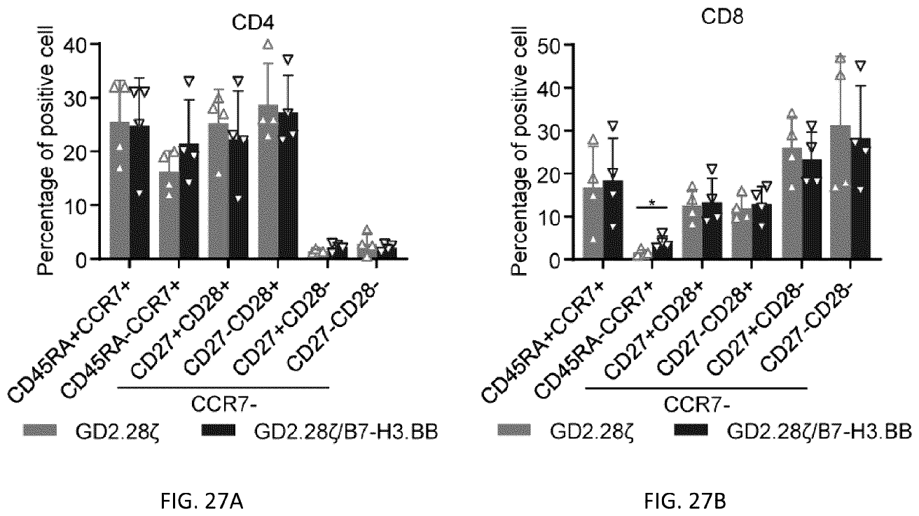

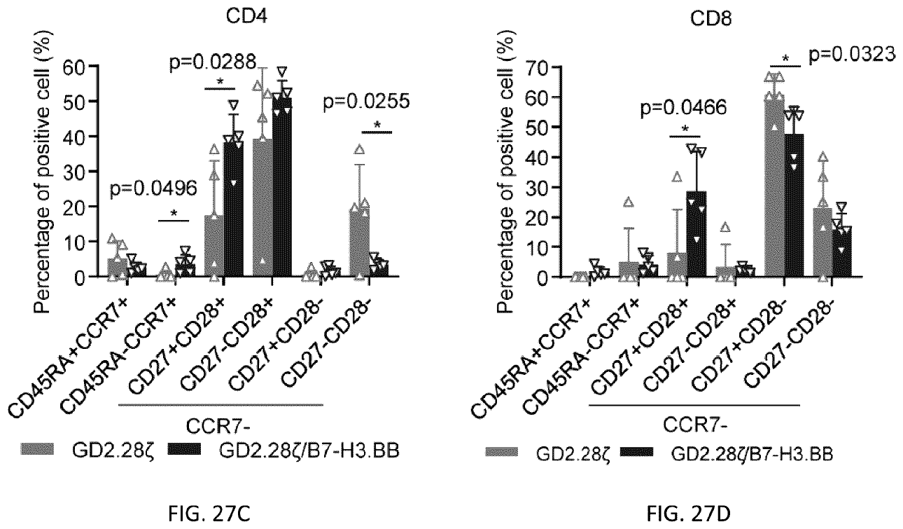

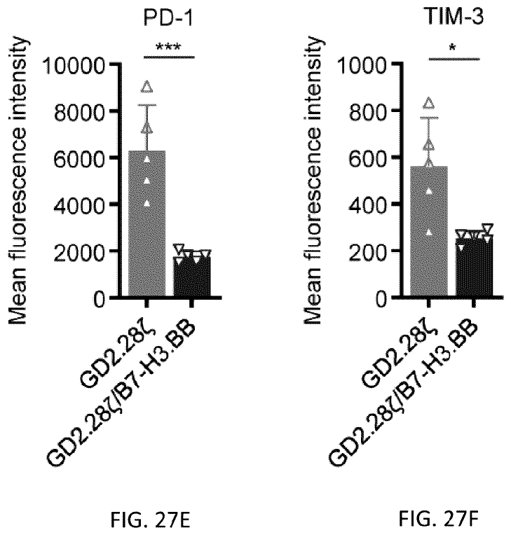

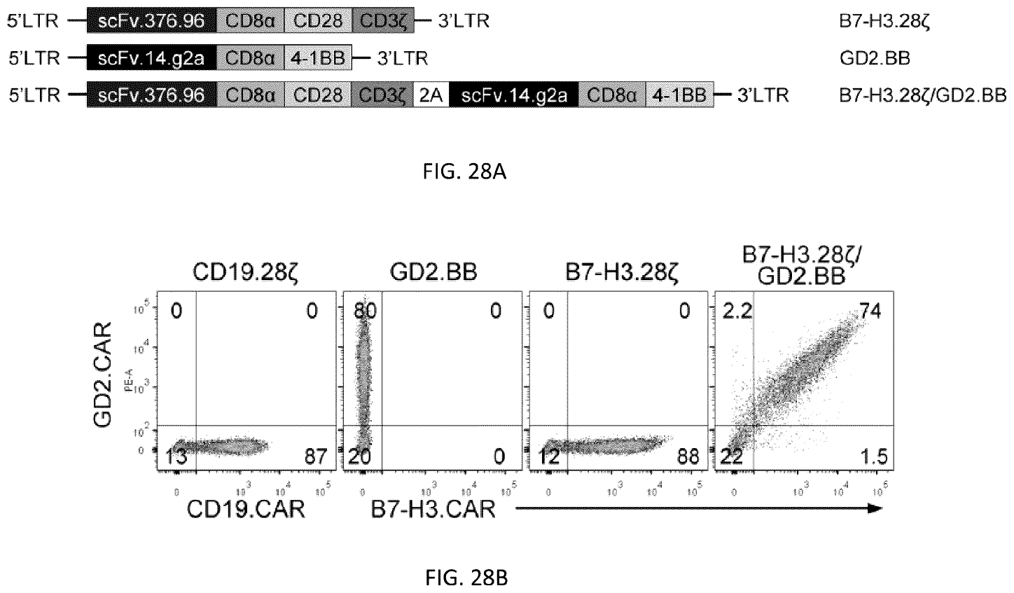

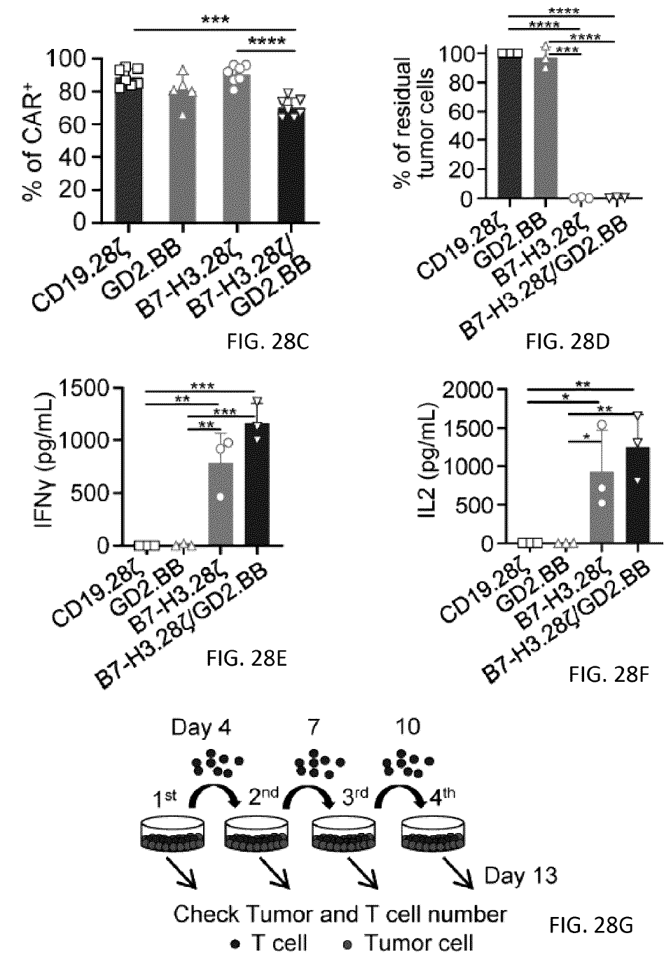

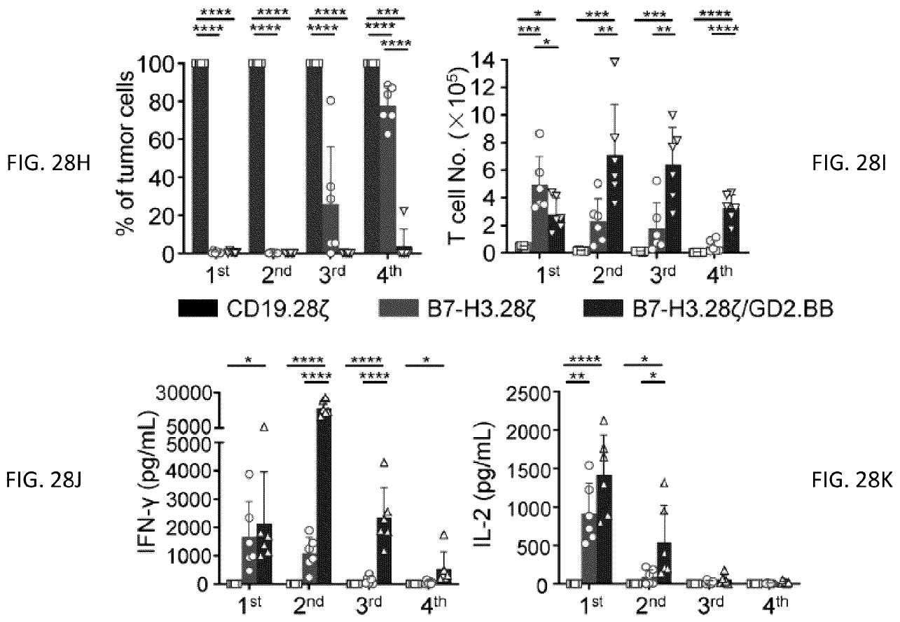

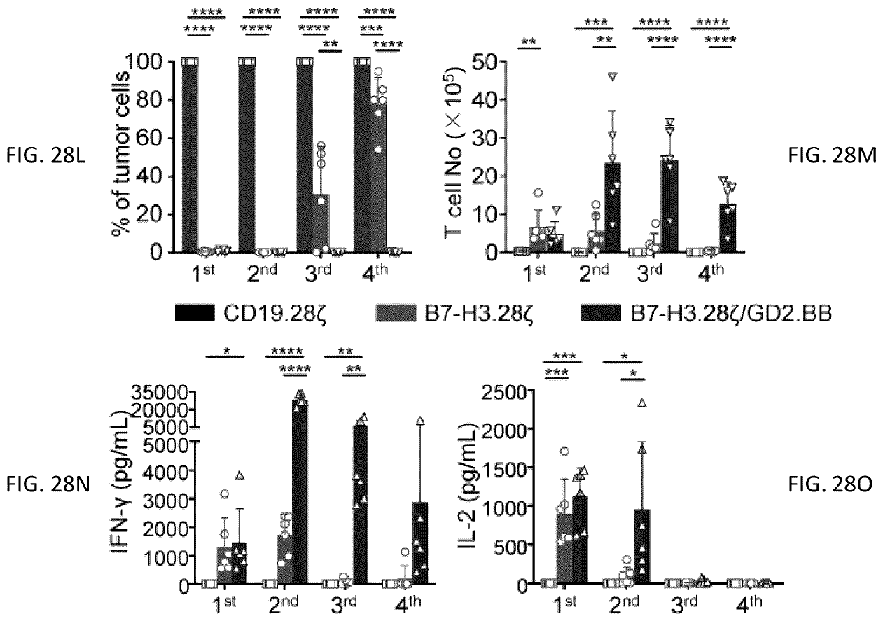

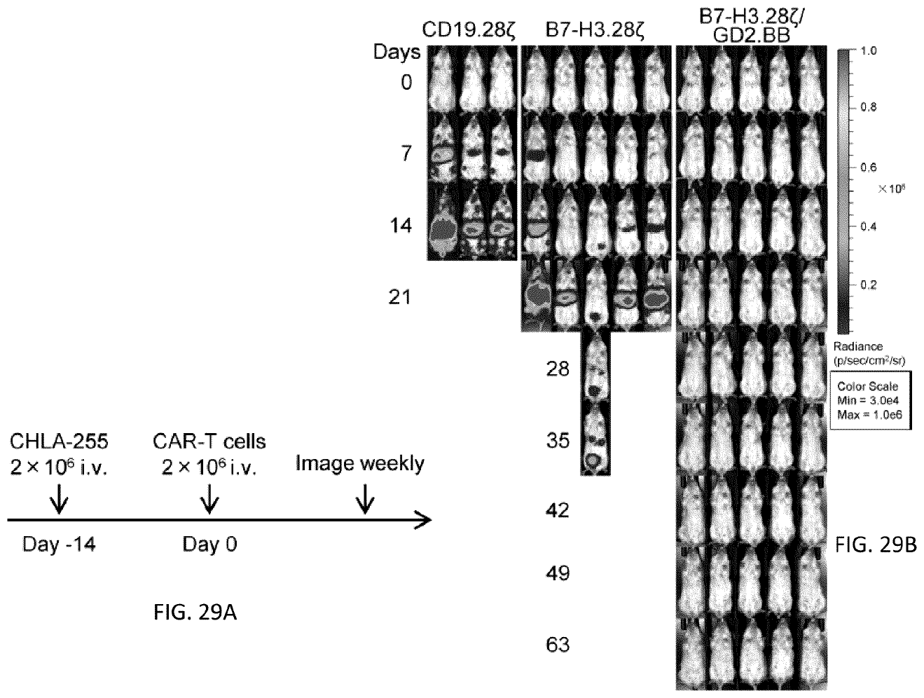

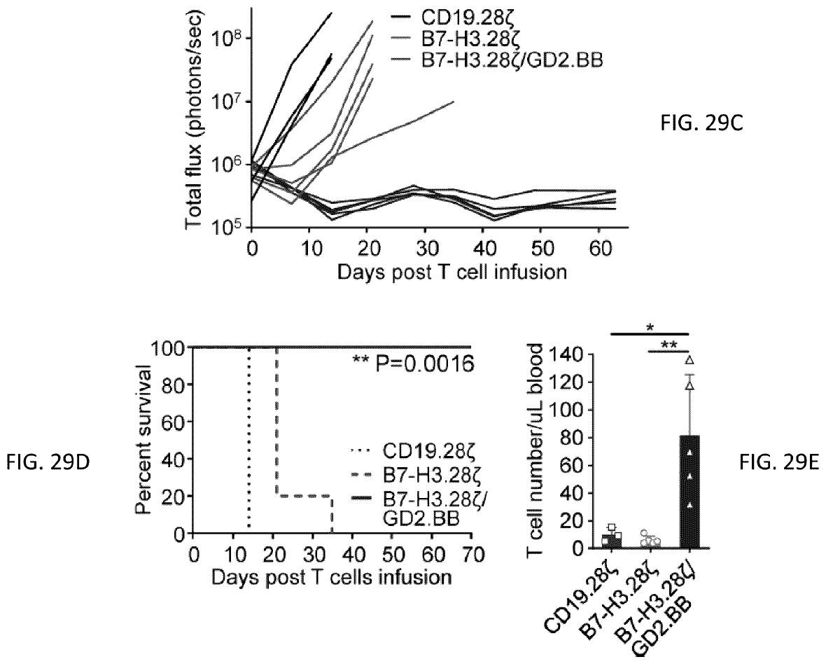

To evaluate if 4-1BB costimulation provided by the B7-H3.BB CAR enhances the antitumor activity of GD2.28ζ CAR-T cells, the functionality of GD2.28ζB7-H3.BB CAR-T cells and GD2.28ζ CAR T cells was compared both in vitro and in vivo. All CARs were found to be expressed in T cells (FIGS. 17A-17B), and no modifications in cell subset compositions were observed (FIGS. 27A-27B). In repetitive multi-round co-culture experiments with NB tumor cells (FIG. 17C), only GD2.28ζB7-H3.BB CAR-T cells continued to eliminate NB cells at the 4^{th} round of co-culture (FIG. 17D, FIG. 17H). In addition, T cells expressing GD2.28ζB7-H3.BB showed the highest T cell counts (FIG. 17E, FIG. 17I) and the highest IFN-γ and IL-2 release (FIGS. 17F-17G, FIGS. 17J-17K) at the 3^{rd} and 4^{h} round of co-cultures. In CHLA-255-bearing NSG mice, GD2.28ζ/B7-H3.BB CAR-T cells not only showed superior antitumor activity to eliminate the primary tumor in stress conditions, but also controlled tumor growth upon tumor re-challenge leading to improved survival (FIGS. 17L-170). At day 14, mice treated with GD2.28ζ/B7-H3.BB CAR-T cells showed the highest frequency of circulating T cells (FIG. 17P), and at day 28 they continued to have a trend of higher circulating T cells (FIG. 17Q). GD2.28ζB7-H3.BB CAR-T cells showed enrichment in CD27+CD28⁺ cells in both CD4⁺ and CD8⁺ T cells, and showed low expression of PD-1 and TIM3 (FIGS. 27C-27F). Also constructed was the dual CAR in which the B7-H3.CAR carries the CD28 costimulation and CD3ζ, while the GD2.CAR contains the 4-1BB costimulatory endo-domain without CD3ζ (B7-H3.28ζ/GD2.BB) (FIG. 28A). It was found that B7-H3.28ζ/GD2.BB CAR-T cells showed superior antitumor activity both in in vitro multi-round co-culture experiments with CHLA-255 and LAN-1 tumor cells (FIGS. 28B-280), and in vivo in the CHLA-255 NB metastatic tumor model (FIG. 29) when compared to single B7-H3.28ζ CAR-T cells.

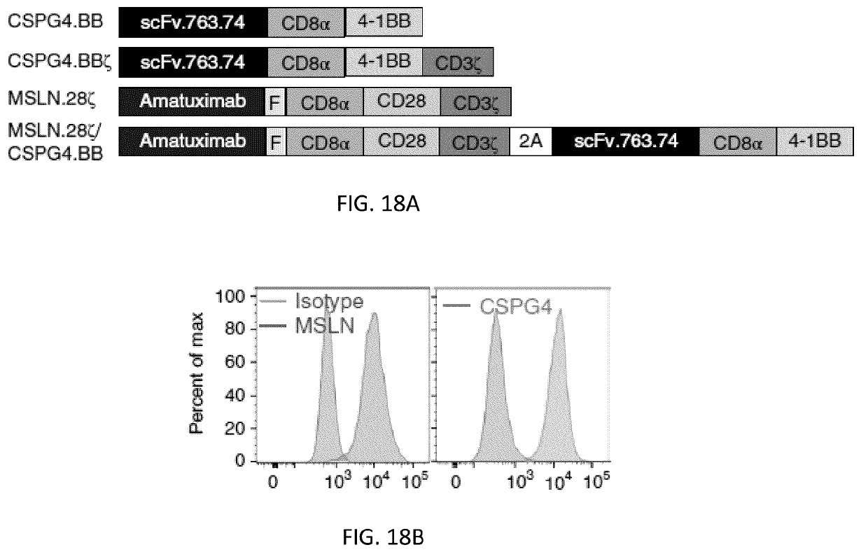

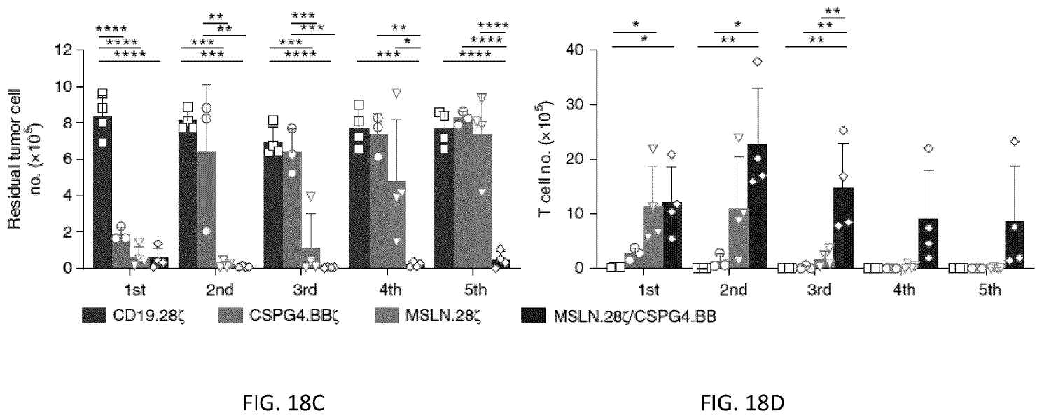

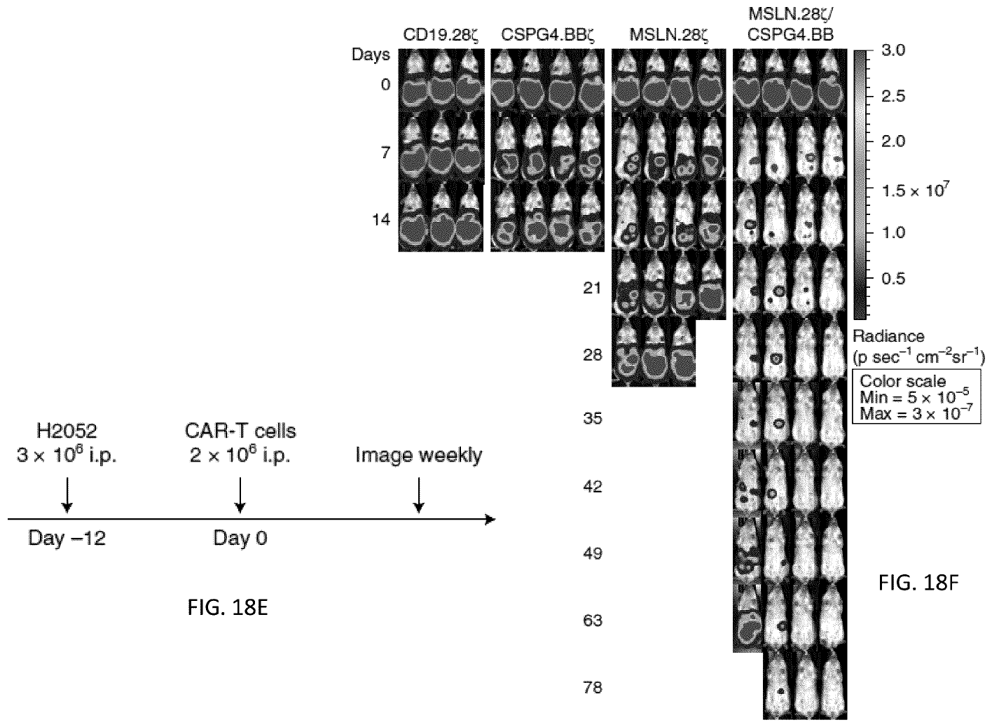

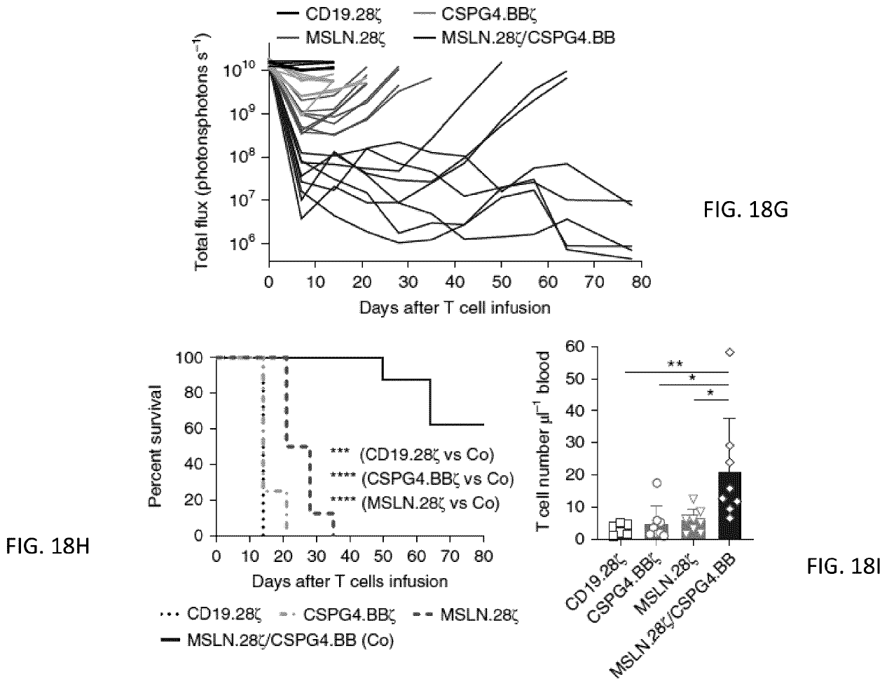

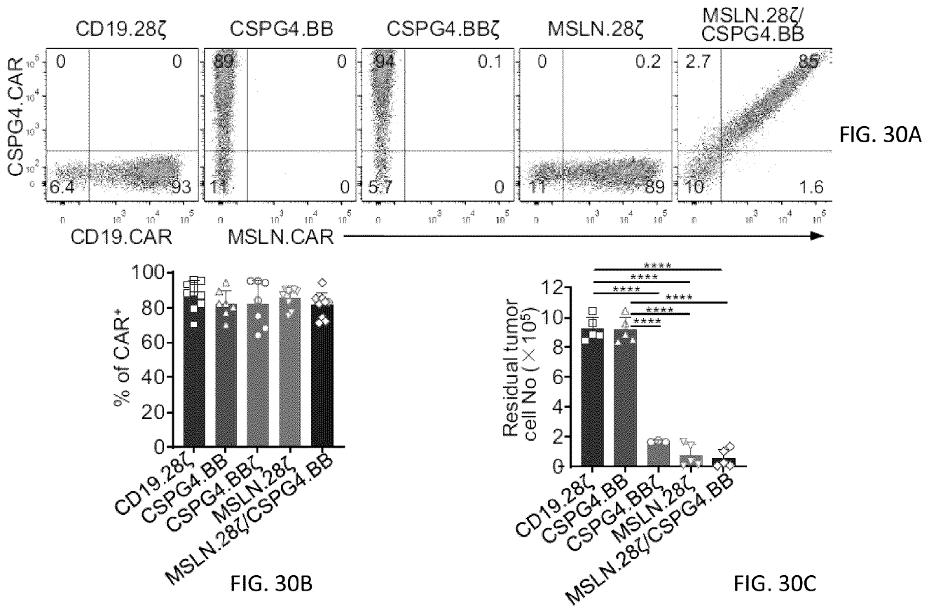

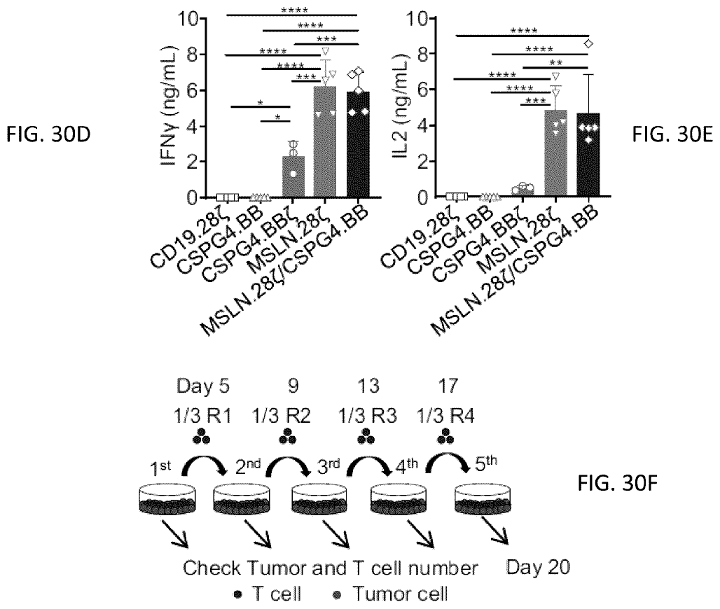

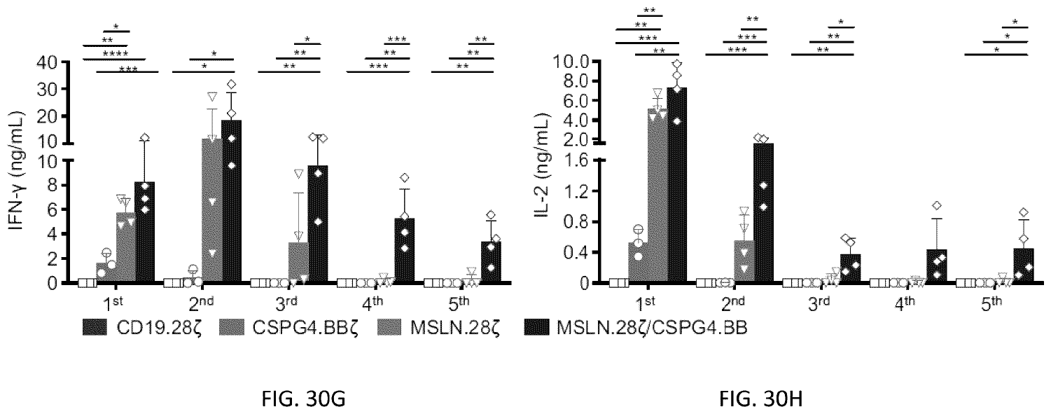

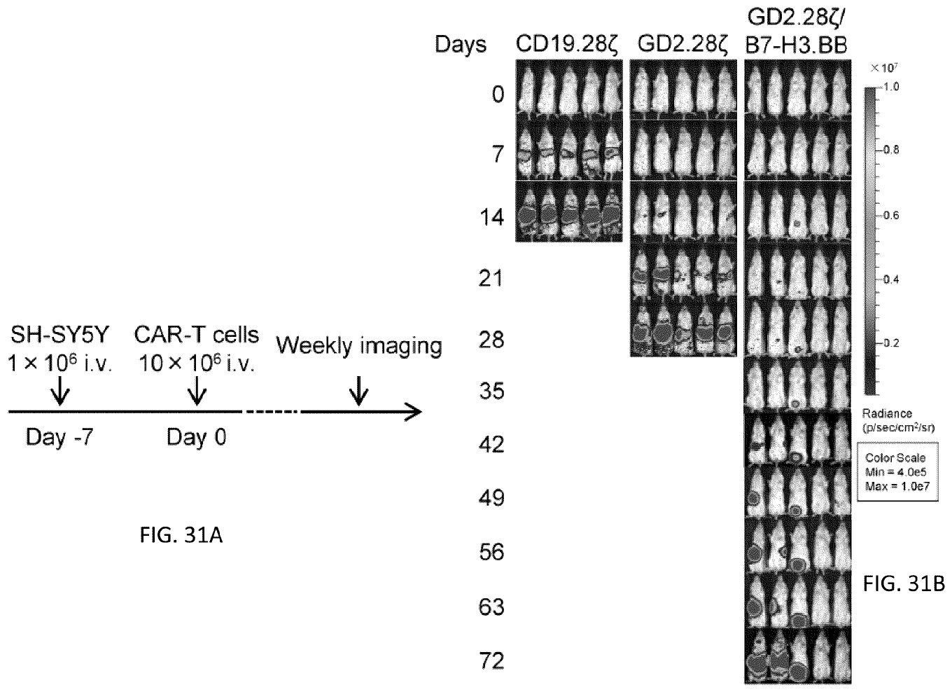

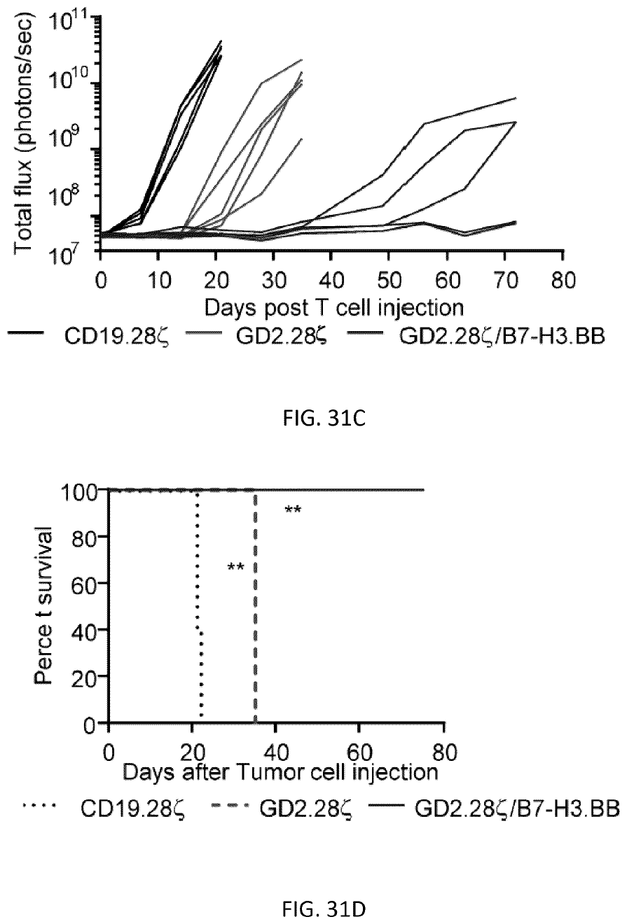

To further investigate if dual targeting, split costimulation and shared CD3ζ can be generally applicable, two additional CARs were utilized targeting mesothelin (MSLN) and chondroitin sulphate proteoglycan 4 (CSPG4)²²⁻²⁴, respectively. A construct was generated encoding the MSLN.CAR with CD28 and CD3ζ domains and co-expressing the CSPG4.CAR with 4-1BB costimulatory domain, but without CD3ζ (MSLN.28ζ/CSPG4.BB) (FIG. 18A). When T cells expressing MSLN.28ζ, CSPG4.BBζ, CSPG4.BBζ, and MSLN.28ζ/CSPG4.BB CARs were co-cultured with the MSLN and CSPG4 double positive mesothelioma cell line H2052 (FIG. 18B), all CAR-T cells with the exception of CSPG4.BB CAR-T cells eliminated the tumor cells and released INF-γ and IL-2 (FIGS. 30A-30E). However, MSLN.28ζ/CSPG4.BB CAR-T cells showed better antitumor activity compared to single target CAR-T cells in multi-round co-culture experiments in vitro (FIGS. 18C-18D and FIGS. 30F-30H), and in an intraperitoneal metastatic mesothelioma model (FIGS. 18E-18I). Overall, these data indicate that dual CAR targeting with split co-stimulatory signal and one single shared CD3ζ domain promotes sustained survival and antitumor effects of CAR-T cells.

### T Cell Activation and Metabolic Fitness of Dual CAR-T Cells

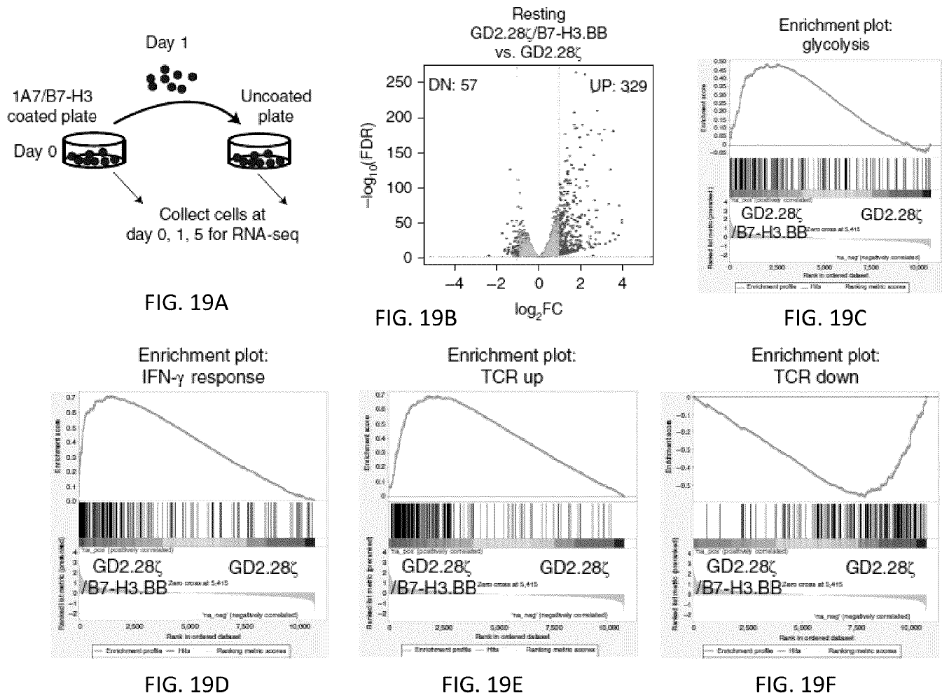

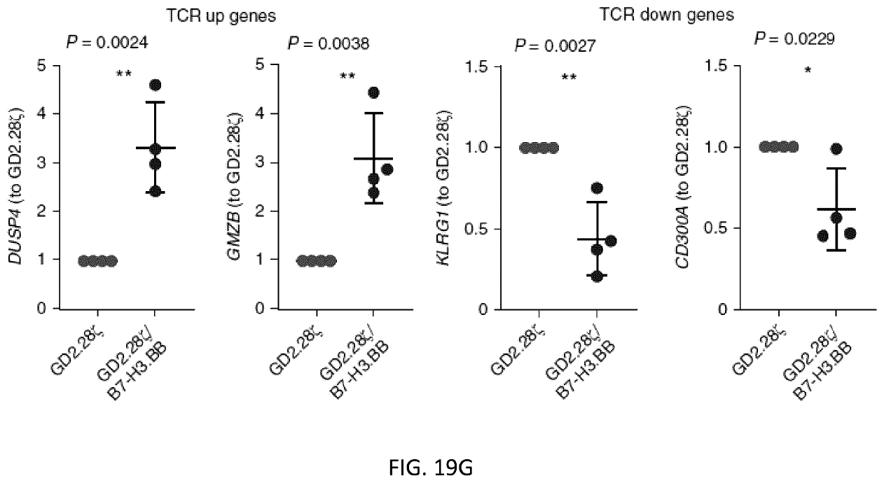

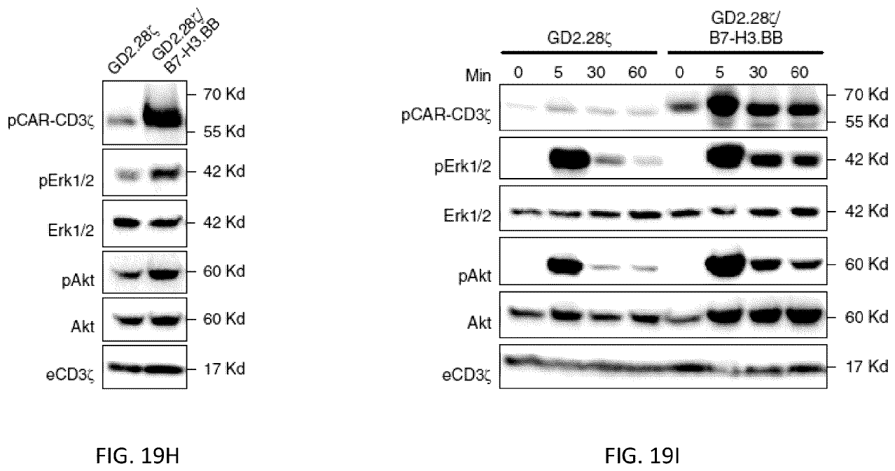

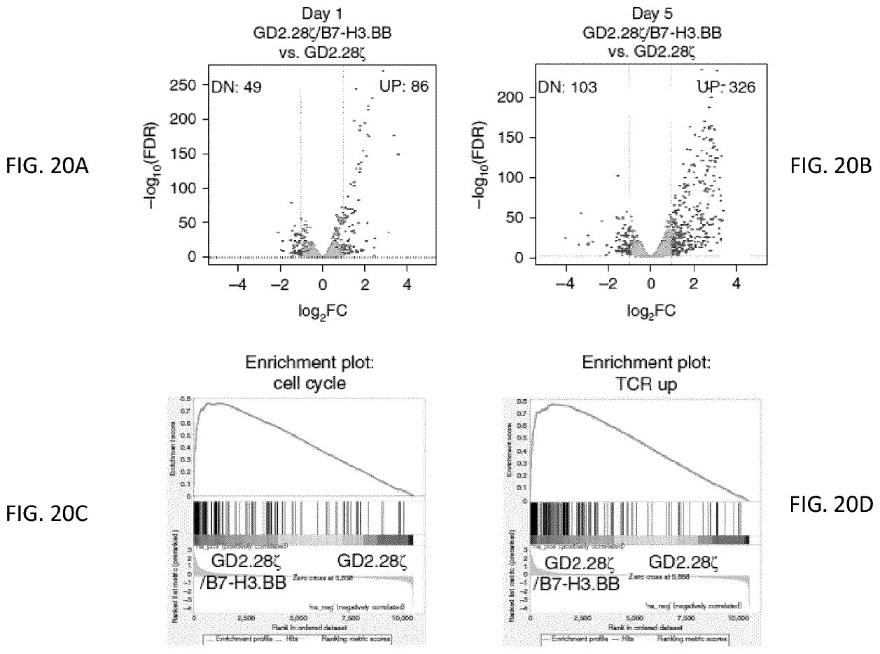

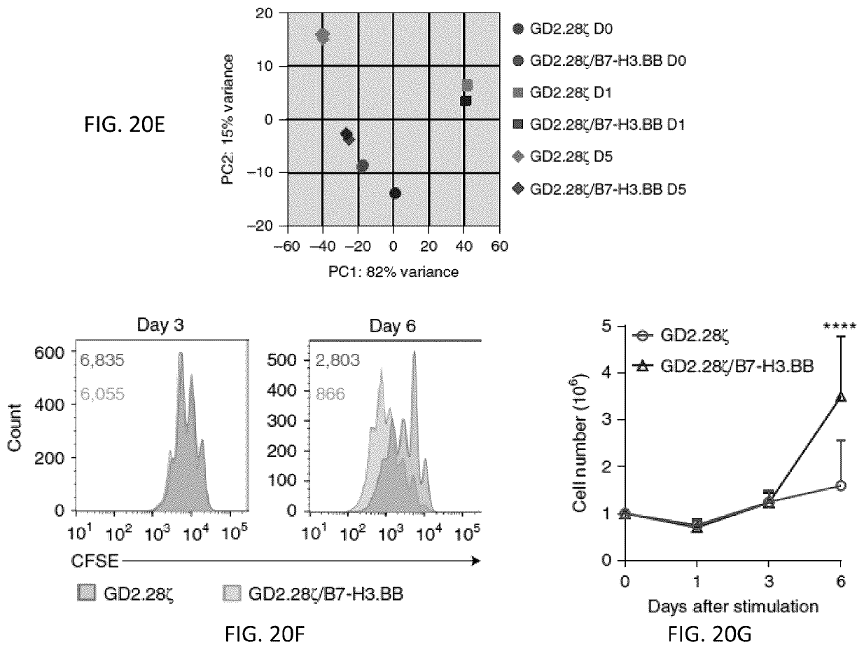

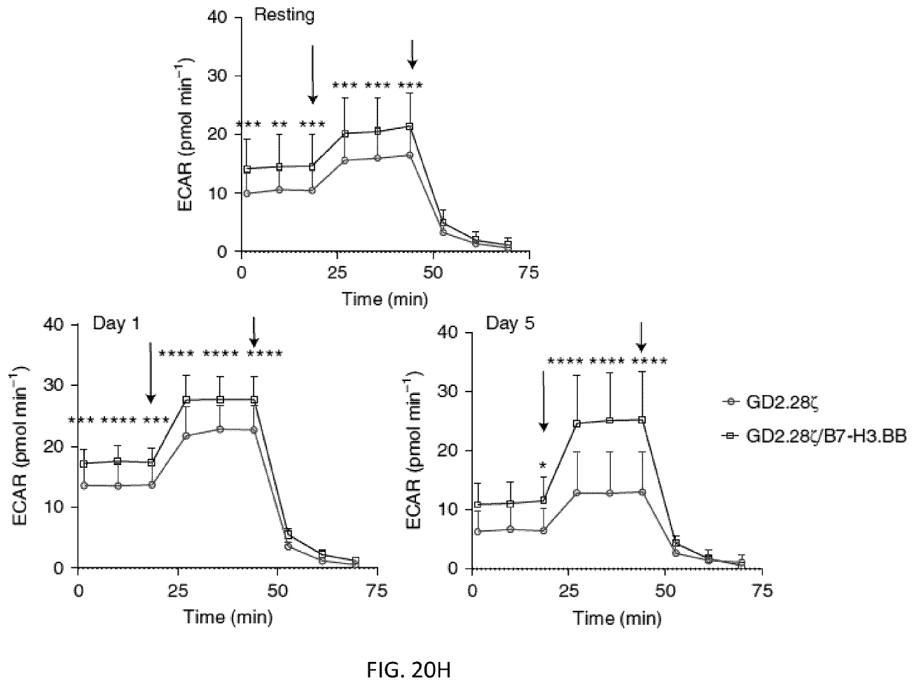

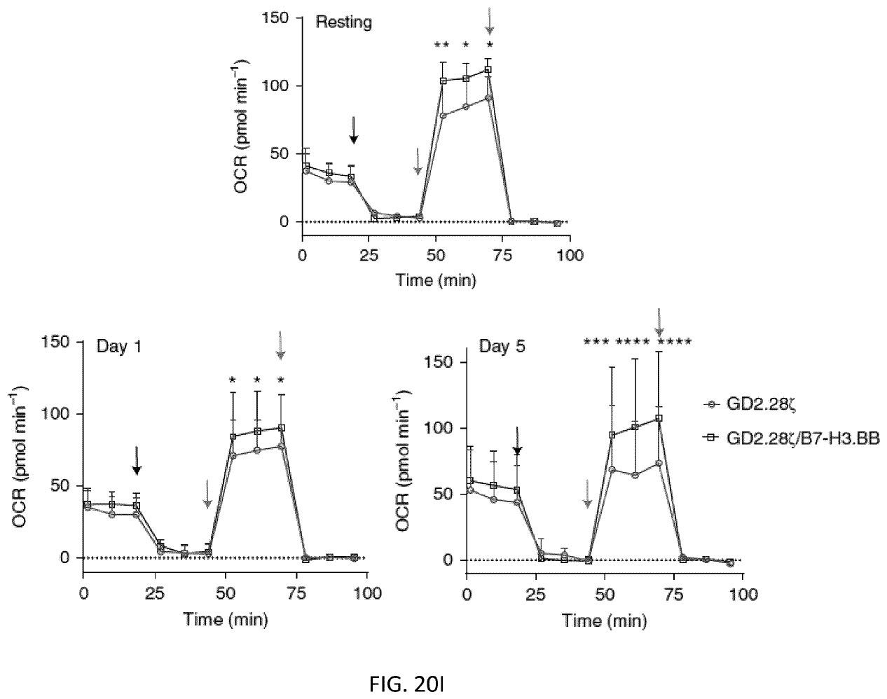

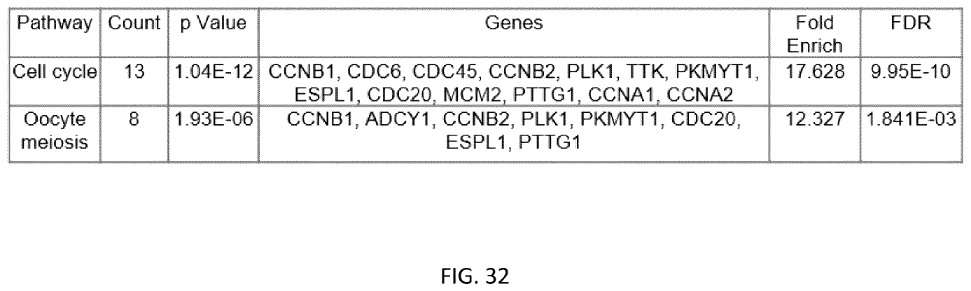

RNASeq analysis was performed to investigate how the addition of B7-H3.BB CAR to the GD2.28ζ CAR enhances antitumor activity and persistence of CAR-T cells. In the absence of CAR engagement, it was found that GD2.28ζB7-H3.BB CAR-T cells showed different gene expression pattern compared to GD2.28ζ CAR-T cells (FIGS. 19A-19B). Gene set enrichment analysis (GSEA) showed that glycolytic pathways and IFN-γ signaling are elevated in GD2.28ζ/B7-H3.BB CAR-T cells (FIGS. 19C-19D). Since both glycolytic and IFN-γ pathways are activated by TCR signaling, the data set was tested using T cell activation gene sets. Transcriptome of CAR-T cells expressing GD2.28ζB7-H3.BB was highly enriched with genes upregulated upon T cell activation as compared to those expressing the GD2.28ζ CAR, while genes downregulated upon T cell activation are enriched in GD2.28ζ CAR-T cells (FIGS. 19E-19G). These data indicate that GD2.28ζB7-H3.BB CAR-T cells have higher basal level of TCR activation signaling, which is paralleled by enhanced phosphorylation of the CAR-CD3ζ chain and downstream signaling kinases such as ERK and Akt (FIG. 19H). It was previously reported that basal CAR-CD3ζ phosphorylation in CAR-T cells encoding the CD28 endodomain primes them to stronger and sustained activation upon CAR cross-linking²⁸. Here it was observed that this effect is further amplified in GD2.28ζB7-H3.BB CAR-T cells compared to T cells expressing the GD2.28ζ CAR alone (FIG. 19I). Despite differences in transcriptome at basal resting condition, initial CAR-mediated stimulation equally activated T cells expressing either the GD2.28ζ/B7-H3.BB CAR or GD2.28ζ CAR as transcriptome profile of T cells with either CARs converged at day 1 (FIG. 2OA). However, gene expression diverged between T cells expressing the GD2.28ζ/B7-H3.BB CAR or GD2.28ζ CAR at day 5 after removal from antigen stimulation (FIG. 20B). CAR-T cells expressing GD2.28ζ/B7-H3.BB showed an enrichment in pathways related to cell cycle and TCR signaling at day 5, suggesting sustained proliferation upon antigen removal (FIGS. 20C-20D). Principle component analysis was performed to determine the relative relationship between the transcriptome of CAR-T cells at day 0, 1 and 5 (FIG. 2OE). The variance of transcriptome of the dataset is dominated by CAR activation, which is captured on PC1 (x-axis). On day 0 and day 5, GD2.28ζ/B7-H3.BB CAR-T cells showed higher PC1 score when compared to GD2.28ζ CAR-T cells, consistent with a finding of active TCR signaling. Notably, CAR-T cells expressing GD2.28ζ/B7-H3.BB acquired additional transcriptome distinction from T cells expressing the GD2.28ζ CAR, which is captured in PC2 (y-axis). When KEGG pathway analysis was performed of the top 100 highest loading genes in PC2, the “Cell Cycle pathway” was found to be the most enriched (FIG. 32). Consistent with the enrichment of cell cycle related pathway, sustained T cell proliferation was found at day 6 post CAR crosslinking of GD2.28ζ/B7-H3.BB CAR-T cells (FIG. 2OF), and better expansion as indicated by almost two fold more T cell counts at day 6 as compared to GD2.28ζ CAR-T cells (FIG. 2OG). On metabolic analyses, CAR-T cells expressing GD2.28ζ/B7-H3.BB showed elevated glycolytic activity as compared to those expressing GD2.28ζ at day 0 and day 5 post-stimulation, while only modest difference were observed at day 1 when activation signaling is at its maximum for T cells expressing either GD2.28ζ/B7-H3.BB CAR or GD2.28ζ CAR (FIG. 2OH). Remarkably, enhanced glycolysis observed in CAR-T cells expressing GD2.28ζ/B7-H3.BB did not disrupt the previously described pro-OXPHOS metabolic profile sustained by 4-1BB endodomain²⁹, because these cells showed significantly increased oxygen consumption rate before or after CAR stimulation as compared to CAR-T cells expressing GD2.28ζ (FIG. 2OI). Overall, transcriptional analysis, T cell signaling and metabolism suggest that dual targeting, split signal and one single CD3ζ domain maintain tonic TCR signaling and metabolic fitness in T cells and translate into potent and sustained antitumor activity.

### Dual CAR-T Cells Prevent Antigen Escape

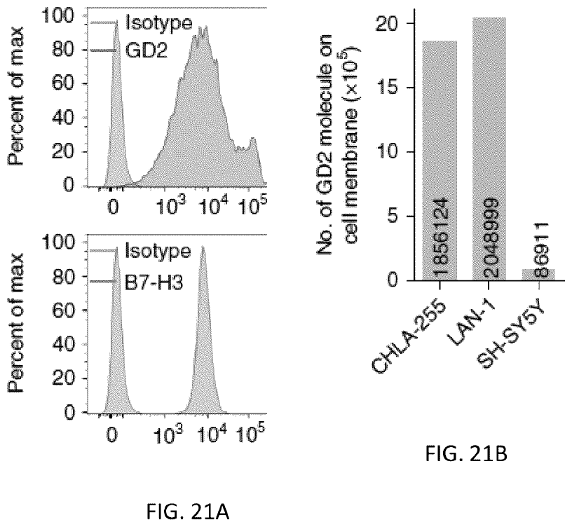

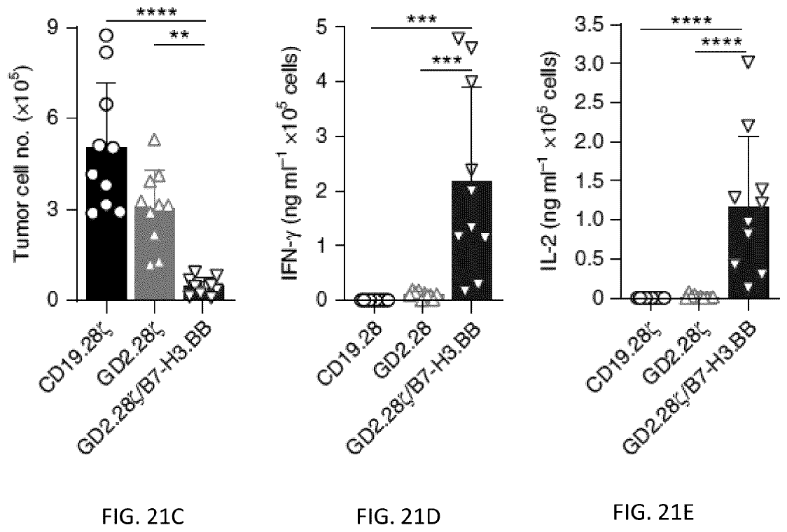

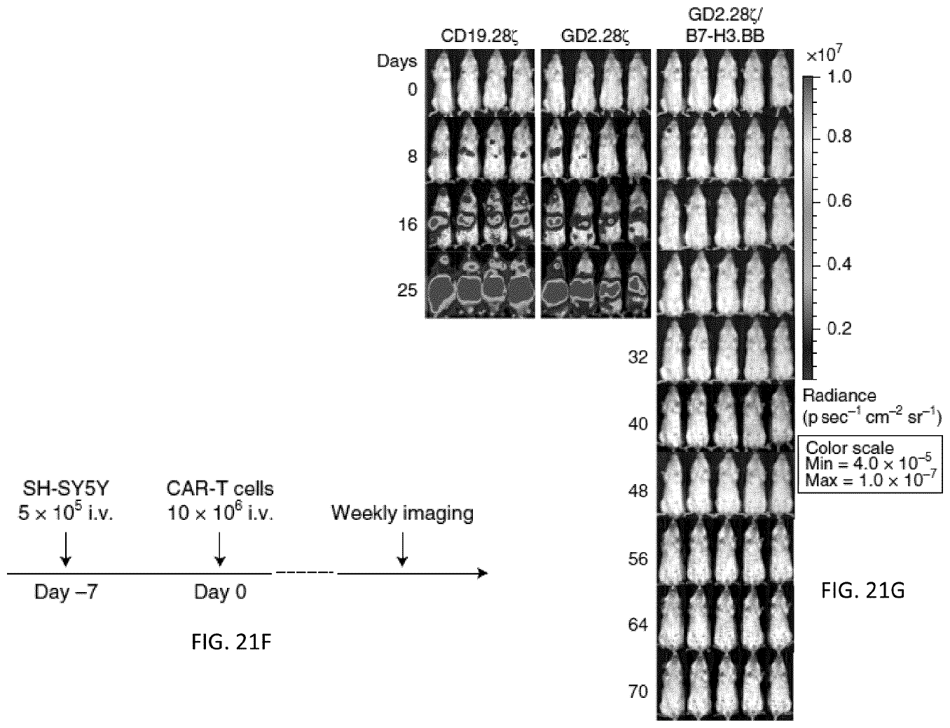

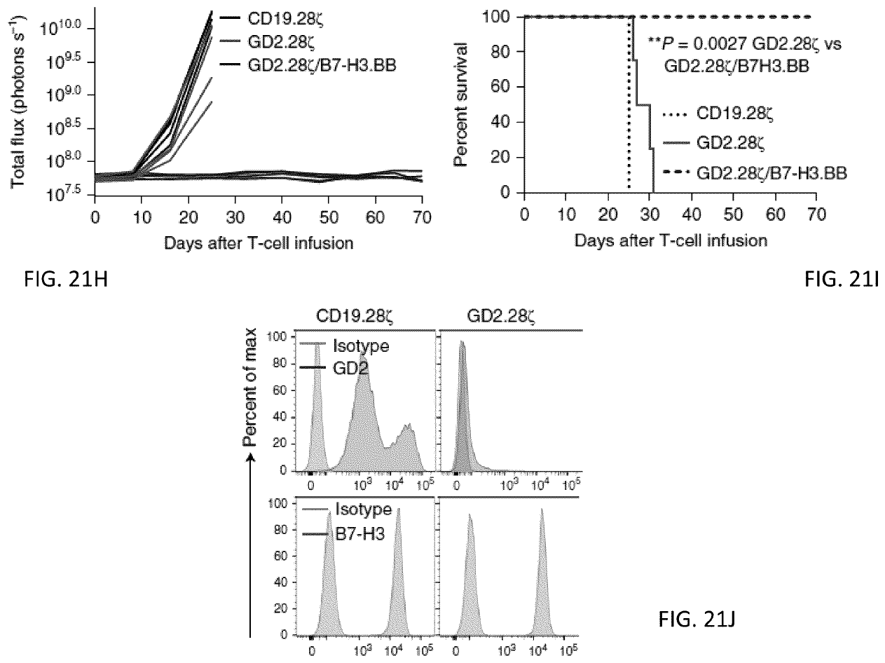

To evaluate if GD2.28ζB7-H3.BB CAR-T cells prevent tumor escape due to variable antigen expression in tumor cells, the heterogeneous levels of GD2 expression in NB were leveraged. CAR-T cells expressing either GD2.28ζ or GD2.28ζB7-H3.BB were cocultured with the NB cell line SH-SY5Y that shows heterogeneous expression of GD2 (FIGS. 21A-21B). GD2.28ζB7-H3.BB CAR-T cells exhibited the highest antitumor effects (FIG. 21C) and Th1 cytokine release (FIGS. 21D-21E). Next, GD2.28ζB7-H3.BB CAR-T cells were evaluated to see if the cells can prevent tumor escape in vivo. In a low tumor burden model in SH-SY5Y-bearing NSG mice (FIG. 21F), CAR-T cells expressing GD2.28ζ/B7-H3.BB fully controlled tumor growth (FIGS. 21G-21I). In mice treated with GD2.28ζ CAR-T cells, growing tumors showed dim GD2 expression as compared to control mice treated with CD19-specific CAR-T cells. In contrast, B7-H3 expression remained unmodified in tumor cells since this antigen was not targeted in mice infused with GD2.28ζ CAR-T cells (FIG. 21J). Furthermore, CAR-T cells expressing GD2.28ζB7-H3.BB effectively controlled the tumor growth in mice treated with higher tumor burden (FIG. 3I). Overall, these data indicate that T cells expressing dual targeting CARs with split costimulatory signal and one single CD3ζ domain have superior antitumor activity when tumor contains cells with dim expression of the targeted antigen, which may cause tumor escape from single targeting CAR-T cell treatment.

### DISCUSSION

Preventing tumor escape due to heterogeneity in antigen expression and providing optimal T cell costimulation remain critical aspects to achieve clinical responses with CAR-T cells in solid tumors. Here, CAR-T cells were generated that simultaneously target two antigens and provide optimal costimulation and T cell metabolic fitness by activating independently CD28 and 4-1BB pathways and tuning CD3ζ-chain-mediated signaling. In a model of NB, T cells expressing the dual CAR provided robust and sustained antitumor activity in in vivo stress conditions and prevented tumor escape due to heterogeneous antigen expression by tumor cells. The beneficial effects of the proposed combination of dual targeting, split costimulation and tuned CD3ζ signaling was reproduced using another pair of CAR molecules.

Targeting GD2 with CAR-T cells hold great clinical potential in NB^{17,19,26-28}. The approach has been proved safe, but the ideal costimulation of GD2-specific CAR-T cells in the clinical setting remains undefined. In particular, the most recent clinical study at Baylor College of Medicine used a 3^{rd} generation CAR encoding both CD28 and OX40 endodomains which demonstrated only modest clinical effects despite the combination with a checkpoint blockade¹⁷. Furthermore, while GD2 is widely recognized as an ideal target for immunotherapy of NB, its heterogeneous expression in tumor cells is not fully appreciated, and will lead to tumor recurrence due to the selection of tumor clones with low GD2 expression²⁹-3². Thus, a second clinically relevant NB target represented by B7-H3²¹ was added. Of note, GD2 and B7-H3 are physiologically expressed by NB, and the possibility to target these antigens simultaneously on tumor cells that naturally express the targets reflecting the physiologic density of antigen expression in tumor cells was tested. Similarly, MSLN is currently under evaluation to treat mesothelioma, lung cancer, breast cancer, pancreatic cancer and prostate cancer via scFv-based CAR-T cells³³, and its optimal combination with another clinically relevant target such as CSPG4³⁴,3⁵ was explored.

The data demonstrate that it is possible to accommodate two almost complete CAR sequences targeting two different antigens into a single retroviral vector and obtain functional expression levels of both CARs in T cells. The first observation made is that T cells expressing two CARs, providing transacting CD28 and 4-1BB endodomains, and each one with its own CD3ζ chain did not show any beneficial effects in in vivo stress conditions. In sharp contrast, T cells expressing the same two CARs in which the CD3ζ chain is provided by one single CAR not only showed cytotoxic effects of each CAR, but also caused antitumour effects in vivo as compared to single targeting in stress conditions. The observation that a CAR lacking its own CD3ζ chain can still provide cytolytic effects to T cells if a second complete CAR is also expressed likely reflects unique characteristics of the synapse formed by the CAR molecules. While a CAR that lacks the CD3ζ cannot efficiently use the endogenous CD3ζ chains despite engaging the antigen, it can efficiently use the CD3ζ chain of a bystander CAR expressed by the same cell. The sharing of the CD3ζ chain to trigger T cell activation is strictly mediated by the presence of two cysteine residues in the CD8α stalk as integral part of the CAR molecules. Furthermore, sharing of the CD3ζ chain only occurs upon antigen engagement, which prevents dimerization of the two CARs if both antigens are not engaged.

The second observation made is that CD28 and 4-1BB pathways transacting and independently activated via two distinct CARs is more effective than the classical in cis expression of 3^{rd} generation CARs. This is reminiscent of the proposed approach to supply 4-1BB ligand to CAR-T cells that encode CD28¹⁶. However, while 4-1BB ligand presentation to T cells co-expressing 4-1BBL and CAR requires cross talk between CAR-T cells, the approach described herein has the significant advantage of providing both 4-1BB and CD28 signaling costimulation independently to each single CAR-T cell once they encounter the tumor. Furthermore, splitting costimulation into two CARs also allows targeting two tumor-associated antigens and to some degree prevent tumor escape.

The optimal stretch of costimulation provided by dual specific CARs with split costimulation and single CD3ζ chain is supported by signaling, molecular and metabolic properties. It has been demonstrated that sustained phosphorylation of the proximal signaling molecules of the CAR caused by the CD28 endodomain imprints CAR-T cells to promote rapid antitumor effects^{11,12,36}. In contrast, 4-1BB signaling in CAR-T cells provides the metabolic fitness characterized by the oxidative metabolism that is critical to sustain CAR-T cell persistence³⁷. CAR-T cells expressing the proposed CAR design are characterized by strong and sustained CAR proximal signaling and molecular signature consistent with TCR tonic signal which are critical in promoting rapid antitumor effects. Similarly, metabolic profiling indicates that dual CAR-T cells display rapid effector function via glycolysis, which is supported by CD28 signaling, but they also preserve oxidative function upon antigen removal, which is a characteristic of 4-1BB costimulation, supporting memory formation and long-term persistence.

In summary, a strategy was designed that addresses simultaneously the most challenging tasks in solid tumors such as generating CAR-T cells that rapidly eliminate the tumor and persist to control tumor growth upon tumor re-challenge. Furthermore, they prevent tumor relapse due to the emergence of tumor cells characterized by low antigen expression.

### Materials and Methods

### Cell Lines and Cell Culture

Human mesothelioma cell line H2052 and human B-cell lymphoma cell line Raji were purchased from American Type Culture Collection (ATCC), Raji-B7-H3 was generated by transducing the Raji cell with retrovirus encoding B7-H3²¹. Human NB cell lines CHLA-255 and Firefly-luciferase (FFLuc)-CHLA-255 are gifts from Dr. Metelitsa at Baylor College of Medicine (Houston, TX) (originally derived from a metastatic lesion in the brain in a patient with recurrent disease at Children's Hospital Los Angeles)^{38,39}, and LAN-1 is a gift from Dr. Brenner at Baylor College of Medicine (Houston, TX)^{40,41}, originally purchased from ATCC. SH-SY5Y is gift from Dr. Chiarle at Boston Children's Hospital (Boston, MA)⁴², originally purchased from ATCC. CHLA-255, LAN-1, H2052 and Raji were cultured in RPMI-1640 (Gibco, Invitrogen) supplemented with 10% fetal bovine serum (Sigma), 2 mM GlutaMAX (Gibco), 100 unit/mL of penicillin (Gibco) and 100 μg/mL of streptomycin (Gibco). SH-SY5Y was cultured in DMEM (Gibco) supplemented with 10% fetal bovine serum (Sigma), 2 mM GlutaMAX (Gibco), 100 unit/mL of penicillin (Gibco) and 100 μg/mL of streptomycin (Gibco). Cells were maintained in a humidified atmosphere containing 5% C02 at 37° C. Tumor cell lines were transduced with a gamma retroviral vector encoding the enhanced green fluorescent protein (eGFP) and/or the FFLuc genes as described previously⁴³. All cell lines were mycoplasma free, and validated by flow cytometry for surface markers and functional readouts as needed.

### Plasmid Construction

All retroviral vectors were generated using the SFG backbone. The GD2-specific CARs were generated using the modified GD2 specific single-chain variable fragment (14G2a scFv), the CD8α stalk and transmembrane domain, the CD28 or 4-1BB intracellular domain, and CD3ζ intracellular domain (GD2.28ζ and GD2.BBζ) as described to correct the previously reported tonic signaling of the scFv²⁶. B7-H3-specific CARs were generated using the 376.96 scFv, the CD8α stalk and transmembrane domain, the CD28 or 4-1BB intracellular domains, and CD3ζ intracellular domain (B7-H3.28ζ and B7-H3.BBζ)¹⁹. The CD19-specific CARs was generated using the FMC63 scFv, the CD8α stalk and transmembrane domain, the CD28 intracellular domain, and CD3ζ intracellular domain (CD19.28ζ)⁴⁴. The cassette encoding the GD2.28ζ in combination with the B7-H3.BB CAR with or without CD3ζ intracellular domain linked with a 2A-sequence peptide (GD2.28ζ/B7-H3.BBζ, and GD2.28ζB7-H3.BBζ) was generated by gene synthesis (GeneArt, Thermo Scientific). The GD2-specific CAR encoding both CD28 and 4-1BB and CD3ζ (GD2.28.BBζ) was generated by gene synthesis (GeneArt, Thermo Scientific). The B7-H3.BB.CAR was generated by PCR using B7-H3.BBζ as template to remove CD3ζ. The cassette encoding the GD2.28ζ and the destabilized human nerve growth factor receptor (dNGFR) with the CD8α stalk and transmembrane domain and 4-1BB intracellular domain was linked with a 2A-sequence peptide (GD2.28ζ/dNGFR.BB). The cassette encoding dNGFR, the CD8α stalk and transmembrane domain, the CD28 intracellular domain, and CD3ζ in combination with the B7-H3.BB CAR domain was linked with a 2A-sequence peptide (dNGFR.287B7-H3.BB). The 287B7-H3.BB cassette was constructed by removing the 14g2a scFv sequence from GD2.28ζ/B7-H3.BB by PCR. The GD2.BB cassette was constructed by removing the CD3ζ from GD2.BBζ by PCR. The B7-H3.28qGD2.BB cassette was constructed by adding GD2.BB to B7-H3.28ζ by gene synthesis (GeneArt, Thermo Scientific) and linked with a 2A sequence. The CSPG4.BBζ CAR was constructed by cloning the scFv of 763.74, CD8α stalk and transmembrane domain, the 4-1BB intracellular domain, and CD3ζ ²³. CSPG4.BB was constructed by removing the CD3ζ from CSPG4.BBζ by PCR. The MSLN.28ζ CAR was constructed by cloning the scFv of amatuximab, CD8α stalk and transmembrane domain, the CD28 intracellular domain, and CD3ζ ⁴⁵. The MSLN.28ζ/CSPG4.BB cassette was constructed by inserting CSPG4.BB into the MSLN.28ζ backbone and linked with the 2A sequence.

### Retrovirus Preparation, Transduction and Expansion of Human T Cells

Retroviral supernatants used for the transduction of human T cells were prepared as previously described⁴³. Buffy coats from healthy donors were purchased from the Gulf Coast Regional Blood Center, Houston, TX. CAR-T cells were generated using peripheral blood mononuclear cells (PBMCs) isolated with Lymphoprep density separation (Fresenius Kabi Norge). PBMCs were activated on plates coated with 1 μg/mL CD3 (Miltenyi Biotec) and 1 μg/mL CD28 (BD Biosciences) agonistic monoclonal antibodies (mAbs). T lymphocytes were transduced on retronectin-coated plates (Takara Bio) and then expanded in complete medium (45% RPMI-1640 and 45% Click's medium (Irvine Scientific), 10% Hyclone FBS (HyClone), 2 mM GlutaMAX (Gibco), 100 unit/mL of Penicillin (Gibco) and 100 μg/mL of streptomycin (Gibco) with IL-7 (10 ng/mL; PeproTech) and IL-15 (5 ng/mL; PeproTech), changing medium every 2-3 days for 12-14 days. T cells were cultured in IL-7/IL-15 depleted medium for two days prior to being used in in vitro functional assays⁴⁶.

### Western Blot Analysis

Protein lysates were normalized according to the percentage of CAR expression and the number of T cell, and were resuspended in 2×Laemelli Buffer (Bio-Rad) in reducing condition (with β-mercaptoethanol). To assess signaling through the CAR, T cells were put on ice and incubated with 2 μg of the 1A7 anti-idiotype mAb specific for the 14G2a scFv and 1 μg of the 41g-B7-H3 fused with mouse IgG2a Fc (Chimerigen Laboratories) for 20 minutes and then cross-linked with 2 μL of goat anti-mouse secondary Ab (BD Bioscience) for an additional 20 minutes. Cells were then transferred to a 37° C. water bath for the indicated time points and lysed with 2×Laemelli Buffer. To assess dimers and oligomers of two CARs, T cells were put on ice and incubated with 1 μg of the 41g-B7-H3 fused with mouse IgG2a Fc (Chimerigen Laboratories) for 20 minutes and then incubated with 2 μL of a goat anti-mouse secondary Ab (BD Bioscience) for an additional 20 minutes. Cells were then transferred to 37° C. water bath for 20 minutes and lysed with 2×Laemelli Buffer in reducing (with β-mercaptoethanol) or non-reducing (without β-mercaptoethanol) conditions for 10 minutes at 100° C. All lysates were separated in 4-15% sodium dodecyl sulfate polyacrylamide gels (SDS-PAGE, Bio-Rad) and transferred to polyvinylidene diflouride membranes (Bio-Rad). After protein transfer onto polyvinylidene diflouride membranes, membranes were blocked in 5% non-fat milk in Tris-buffered saline, 0.1% Tween 20 (TBST) and incubated with primary at a dilution of 1 to 1,000 in TBST with 5% non-fat milk. The following abs were used: anti-phospho-CD3ζ (Y142) (Abeam), anti-CD3ζ (Santa Cruz Biotechnology), anti-ERK (pan) (BD Biosciences), anti-phospho-Akt (Ser473) (Cell Signaling), and anti-Akt (pan) (Cell Signaling). Membranes were then incubated with HRP-conjugated goat anti-mouse or goat anti-rabbit IgG (both Santa Cruz) at a dilution of 1 to 3,000 and developed with SuperSignal West Femto Maximum Sensitivity Substrate or SuperSignal West Pico Chemiluminescent Substrate (both Thermo Scientific) on the Gel station (Boi-Rad).

### Enzyme-Linked Immunosorbent Assay (ELISA)

Culture supernatants were collected after 24 hours of co-culture to measure the release of IFN-γ and IL-2 by using specific ELISA kit (R&D systems) following manufacturer's instructions. Each supernatant was measured in duplicate. Samples were measured using Synergy 2 Multi-Detection Microplate Reader and Gen5 software (both BioTek).

### Flow Cytometry

Flow cytometry was performed using Abs specific to human CD3, CD4, CD8, CD27, CD28, CD45, CD45RA, CCR7, PD-1, LAG-3, TIM-3, B7-H3 (clone 7-517), and GD2 (clone 14G2a) (all from BD Biosciences) conjugated with phycoerythrin (PE), PE-Cy7, fluorescein isothiocyanate (FITC), allophycocyanin (APC), APC-H7, Alexa Fluor 647 (AF 647), Brilliant Violet (BV) 421, BV510, BV605, and/or BV711. Expression of human B7-H3 in tumor cell lines was assessed with the 376.96 mAb and confirmed with another B7-H3 Ab clone 7-517 (BD Biosciences)⁴⁷. To detect CD19.CAR, GD2.CAR and CSPG4.CAR, the anti-idiotype mAbs 233-4A, 1A7 and MK2-23 were used as primary Ab to stain the CARs respectively, and then followed by either APC or PE conjugated rat anti-mouse IgG secondary mAb (BD Biosciences). MSLN.CAR has a fused flag tag, therefore, it's expression was detected by using the APC-conjugated Anti-Flag mAb (Clone L5, Biolegend). The expression of B7-H3.CAR was detected using the recombinant human B7-H3 Fc chimera protein (R&D systems) followed by the Alexa Fluor 647-conjugated AffiniPure F(ab′)2 Fragment Goat Anti-Human IgG (H+L) secondary mAb (Jackson ImmunoResearch). For absolute number calculations, samples were analyzed using CountBright absolute counting beads (Thermo Scientific). Samples were analyzed with BD FACScanto II or BD FACSfortessa (BD Biosciences) with the BD Diva software (BD Biosciences), for each sample a minimum of 10,000 events was acquired, and data was analyzed using Flowjo 10 (Tree Star). For the carboxyfluorescein diacetate succinimidyl ester (CSFE)-based proliferation assay, T cells were labeled with 1.5 mM CSFE (Invitrogen) and plated with tumor cells at the T cell to tumor cell ratio of 1 to 1. CFSE dilution was measured on gated T cells on day 5 using flow cytometry⁴⁸.

### Measurement of the GD2 Antigen Density

The determination of GD2 antigen density on the cell surface of CHLA-255, LAN-1 and SH-SY5Y was performed using both DAKO QIFIKIT (BIOCYTEX, Glostrup, Denmark) and primary antibodies specific to GD2 (clone 14G2a) and B7-H3 (clone 376.96). All procedure was performed according to the manufacture's recommended protocol. The intensity of fluorescence was correlated with the number of the bounded primary antibody molecules on the surface of the cell lines. Antigen density was calculated based on the MFI of the stained cells with the standard curve that made by using the MFI of five populations of beads bearing different numbers of mouse monoclonal antibody molecules.

### Long-Term In Vitro Cytotoxicity and Multi-Round Co-Culture Assay

Tumor cells were seeded in 24-well plates at a concentration of 5×10⁵ cells/well one day prior to the addition of T cells. T cells normalized for transduction efficiency were added at the T cell to tumor cell ratio of 1 to 5 without the addition of exogenous cytokines. On day 5 of co-culture, cells were collected and T cells and tumor cells were measured by flow cytometry based on CD3 and GFP expression, respectively. Supernatant were also collected for cytokine measurements 24 hours after each cycle start. CD19.28ζ.CAR-Ts were used as negative control. Dead cells were gated out by Zombie Aqua Dye (Biolegend) staining. For multiple rounds of co-culture with NB cell lines of CHLA-255 and LAN-1, tumor cells were seeded at 5×10⁵ per well in 24-well plates one day prior to the addition of T cells. T cells normalized for transduction efficiency were added at the T cell to tumor cell ratio of 1 to 5. Four, three and two duplicates were performed for the 1^{st}, 2^{nd} and 3^{rd} round of co-culture for each condition, respectively. At the end of each round of co-culture, one duplicate was harvested for quantifying residual tumor cells (GFP+) and T cells (CD3⁺) by flow cytometry using CountBright absolute counting beads (Thermo Scientific), and T cells in other duplicates were collected and used for next round of co-culture. In the co-culture of GD2.28ζ/B7-H3.BB vs. GD2.28ζ CAR-T cells, at day 4, 6, and 8, T cells were harvested and transferred into a new well in which 5×10⁵ NB cells were seeded one day before of adding T cells for the next round of co-culture. In the co-culture of B7-H3.28ζ/GD2.BB vs. B7-H3.28ζ CAR-T cells, at days 4, 7, and 10, T cells were harvested and transferred into a new well in which 5×10⁵ NB cells were seeded one day before of adding T cells for the next round of co-culture. Supernatants were also collected for cytokine measurements 24 hours after adding T cells for each round of co-culture. For multiple rounds of co-culture with the mesothelioma cell line H2052, tumor cells were seeded at 2.5×10⁵ per well in 24-well plates one day prior to adding T cells. T cells normalized for transduction efficiency were added at the T cell to tumor cell ratio of 1 to 5. Two duplicates were performed for each round of co-culture. At the end of each round of co-culture, day 5, 9, 13 and 17, one duplicate was harvested for examining residual tumor cells (GFP+) and T cells (CD3⁺) by flow cytometry using CountBright absolute counting beads (Thermo Scientific). In another duplicate T cells were collected and 1/3 were transferred into a new well in which 2.5×10⁵ H2052 cells were seeded one day before of adding T cells for the next round of co-culture. Supernatant was also collected for cytokine measurements 24 hours after adding T cells for each round of co-culture. For each co-culture experiment, CD19.28ζ.CAR-T cells were used as negative control. Dead cells were gated out by Zombie Aqua Dye (Biolegend) staining while T cells were identified by the expression of CD3 and tumor cells by the expression of GFP.

### Reverse Transcription Quantitative Polymerase Chain Reaction

Cells were lysed and RNA was extracted using the RNeasy Minikit (Qiagen) and reverse transcribed into cDNA (Superscript VILO, Invitrogen). Human DUSP4, KLRG1, CD300A, and GZMB mRNA expression was quantified using TaqMan probes (Thermo Scientific) on a QuantStudio 6 PCR machine (Applied Biosystems) using 18S ribosomal RNA as housekeeping gene control (Invitrogen).

### RNA-Sequence Analysis

Briefly, total RNA was extracted from CAR-T cells, and mRNA libraries were prepared (TruSeq Stranded mRNA Library Prep, Illumina) and sequenced on the Illumina HiSeq4000 platform (UNC High-Throughput Sequencing Facility) using paired-end 100-bp reads, with 84 million reads on average (range, 49-139 million).

RNA-seq data were aligned with STAR alignment (v2.4.2) and quantified with Salmon (v0.6.0). Differential gene-expression analysis was performed using the R DESEq2 package⁴⁹. Among all significantly expressed genes between GD2.28ζ-CAR-T cells and GD2.28ζ plus B7-H3.BB.CAR-T cells (FDR p<0.05), expression was further filtered to genes contained within the IFN-γ and the pathway signatures.

Activation of CAR-T Cells for RNA-Seq, qPCR, and Cell Metabolic Assay

One mg/mL of the 1A7 anti-idiotype mAb specific for the 14G2a scFv and 1 g/ml of the 41g-B7-H3 fused with mouse Fc (Chimerigen Laboratories) were coated on non-tissue culture treated 24-well plates for 16 hours in the cold room. Plates were washed with DPBS (Coming) three times before plating T cells (1×10⁶ cells/well). CAR-T cells were incubated on 1A7 mAb and 41g-B7-H3-coated plates at 37° C. for 24 hours. Then, CAR-T cells were transferred to 24-well tissue culture plate. T cell metabolism was measured in a Seahorse XFe24 analyzer (Seahorse Bioscience). 5×10⁵ T cells were seeded to 24 well Seahorse XF-24 assay plates in Seahorse BASE media with additives. Cells were incubated at 37° C. in a non-C02 incubator for 45 min. All media was adjusted to pH 7.4 on the day of assay. Mitochondrial and glycolysis stress tests were performed according to the manufacturer's protocol. Oxygen consumption and extracellular acidification rates were automatically calculated and recorded by the Seahorse XF-24 software.

### Confocal Microscopy

T cells expressing the GFP-tagged GD2.28ζ CAR and B7-H3.BB CAR were stimulated with either the anti-14g2a idiotypic antibody (1A7) or 21g-B7-H3 fused with human IgG1-Fc (R&D), and then crosslinked with a rat anti-mouse IgG1 secondary antibody (BD Biosciences) or Alexa Fluor 647 conjugated Goat-anti-human IgG secondary antibody (Jackson ImmunoResearch Laboratories Inc.) respectively. For the double CAR cross-link, CAR-T cells were stained with 1A7 and 21g-B7-H3-Fc simultaneously, followed by cross-linking with both above corresponding secondary antibodies. All the primary staining were performed at room temperature for 10 minutes, and the secondary cross-link was performed at 37° C. for 15 minutes. Then the CAR-T cells were fixed with cytofix buffer (BD Biosciences), stained with DAPI (Invitrogen) according to the manufacture's protocol, washed with PBS and and mounted on Glass Bottom Microwell Dishes (MatTek corporation). Data acquisition was performed on LSM700 Zeiss laser scanning confocal microscope (objective lens 63X/1.4 Plan Apo Oil, pixel size 0.07 μm, pinhole size 1 AU) using ZEN software (ZEISS Microscopy). All groups of images were acquired with the same settings. Data analysis was performed with Fiji software.

### Xenograft Mouse Models

Mouse experiments were performed in accordance with the University of North Carolina (UNC) animal husbandry guidelines according to protocols approved by the UNC institutional animal care and use committee. Mice were maintained under specific-pathogen-free conditions with daily cycles of 12h light—12h darkness, and continuous health monitoring was carried out on a regular basis. Animals were euthanized upon showing symptoms of clinically overt disease (not feeding, lack of activity, abnormal grooming behavior, hunched back posture) or excessive weight loss (15% body-weight loss over a week). For these studies, 8-10 weeks old female NSG mice (NOD.Cg-Prkdc^{scid}IL2rg^{tm1Wjl}/SzJ, UNC Animal Studies Core Facility) received 2×10⁶ of FFLuc-CHLA-255, 4×10⁶ of FFLuc-LAN-1, 0.5×10⁶ or 1×10⁶ of FFLuc-SH-SY5Y intravenously via tail vein. Seven or fourteen days after tumor cell injection, 2 or 6×10⁶ CAR-positive T cells were injected intravenously via tail vein. For the mesothelioma intraperitoneal metastatic xenograft model, 3×10⁶ FFLuc-labelled H2052 cells were suspended in 50 μL PBS and mixed with 50 μL of Matrigel (Coming) and implanted by intraperitoneal injection. Mice were then treated with CAR-T cells at day 12 after tumor implant via intraperitoneal injection. Tumor growth was monitored by bioluminescence imaging using the IVIS lumina II in vivo imaging system (PerkinElmer) or AMI Optical in vivo imaging system (Spectral instruments imaging). Mice were bled at specific intervals (10-15 days, as per UNC-IACUC guidelines) to measure T cell frequency and/or phenotype. Mice were euthanized when signs of discomfort were detected by the investigators or as recommended by the veterinarian who monitored the mice three times a week, or when the tumor bioluminescence signal was over 10¹⁰ photons s⁻¹. The maximal tumor burden was not exceeded for all mice. At the time of euthanasia, blood and spleen were isolated and analyzed to detect CAR-T cells. In tumor re-challenge experiments, tumor-bearing mice were further injected intravenously via tail vein with 1×10⁶ of FFLuc-CHLA-255 cells.

### Statistical Analyses

Data were summarized as mean+SD as noted in the figure legends. For multiple group comparisons, one-way analysis of variance (ANOVA) was used to determine statistically significant differences between groups, Tukey's multiple comparison test adjusted p value <0.05 indicates a significant difference. Differences between two groups were determined by two-sided unpaired t test, p<0.05 indicates a significant difference. Differences between the survival curves were analyzed by the Log-rank test using GraphPad Prism v8. Experimental sample numbers (n) are indicated in the figure legends. The statistical analysis method is also described in the individual figure legends. Graph generation and statistical analyses were performed using the GraphPad Prism software (GraphPad Software).

### REFERENCES

1 Fucá, G., Reppel, L., Landoni, E., Savoldo, B. & Dotti, G. Enhancing Chimeric Antigen Receptor T-Cell Efficacy in Solid Tumors. Clin Cancer Res 26, 2444-2451, doi:10.1158/1078-0432.Ccr-19-1835 (2020).
2 June, C. H. & Sadelain, M. Chimeric Antigen Receptor Therapy. N Engl J Med 379, 64-73, doi:10.1056/NEJMra1706169 (2018).
3 Grada, Z. et al. TanCAR: A Novel Bispecific Chimeric Antigen Receptor for Cancer Immunotherapy. Mol Ther Nucleic Acids 2, e105, doi:10.1038/mtna.2013.32 (2013).
4 Ahn, S. et al. Cancer Immunotherapy with T Cells Carrying Bispecific Receptors That Mimic Antibodies. Cancer Immunol Res 7, 773-783, doi:10.1158/2326-6066.Cir-18-0636 (2019).
5 Zah, E., Lin, M. Y., Silva-Benedict, A., Jensen, M. C. & Chen, Y. Y. T Cells Expressing CD19/CD20 Bispecific Chimeric Antigen Receptors Prevent Antigen Escape by Malignant B Cells. Cancer Immunol Res 4, 498-508, doi:10.1158/2326-6066.Cir-15-0231 (2016).
6 Qin, H. et al. Preclinical Development of Bivalent Chimeric Antigen Receptors Targeting Both CD19 and CD22. Mol Ther Oncolytics 11, 127-137, doi:10.1016/j.omto.2018.10.006 (2018).
7 Wang, G. et al. Fully human antibody VH domains to generate mono and bispecific CAR to target solid tumors. J Immunother Cancer 9, doi:10.1136/jitc-2020-002173 (2021).
8 Miller, B. R. et al. Stability engineering of scFvs for the development of bispecific and multivalent antibodies. Protein Eng Des Sel 23, 549-557, doi:10.1093/protein/gzq028 (2010).
9 Maude, S. L., Teachey, D. T., Porter, D. L. & Grupp, S. A. CD19-targeted chimeric antigen receptor T-cell therapy for acute lymphoblastic leukemia. Blood 125, 4017-4023, doi:10.1182/blood-2014-12-580068 (2015).
10 Brentjens, R. J. et al. CD19-targeted T cells rapidly induce molecular remissions in adults with chemotherapy-refractory acute lymphoblastic leukemia. Sci Transl Med 5, 177ra138, doi:10.1126/scitranslmed.3005930 (2013).
11 Zhao, Z. et al. Structural Design of Engineered Costimulation Determines Tumor Rejection Kinetics and Persistence of CAR T Cells. Cancer Cell 28, 415-428, doi:10.1016/j.ccell.2015.09.004 (2015).
12 Sun, C. et al. THEMIS-SHP1 Recruitment by 4-1BB Tunes LCK-Mediated Priming of Chimeric Antigen Receptor-Redirected T Cells. Cancer Cell 37, 216-225.e216, doi:10.1016/j.ccell.2019.12.014 (2020).
13 Carpenito, C. et al. Control of large, established tumor xenografts with genetically retargeted human T cells containing CD28 and CD137 domains. Proc Natl Acad Sci USA 106, 3360-3365, doi:10.1073/pnas.0813101106 (2009).
14 Pule, M. A. et al. A chimeric T cell antigen receptor that augments cytokine release and supports clonal expansion of primary human T cells. Molecular therapy: the journal of the American Society of Gene Therapy 12, 933-941, doi:10.1016/j.ymthe.2005.04.016 (2005).
15 Ramos, C. A. et al. In Vivo Fate and Activity of Second—versus Third—Generation CD19-Specific CAR-T Cells in B Cell Non-Hodgkin's Lymphomas. Molecular therapy: the journal of the American Society of Gene Therapy 26, 2727-2737, doi:10.1016/j.ymthe.2018.09.009 (2018).
16 Stephan, M. T. et al. T cell-encoded CD80 and 4-1BBL induce auto- and transcostimulation, resulting in potent tumor rejection. Nat Med 13, 1440-1449, doi:10.1038/nm1676 (2007).
17 Heczey, A. et al. CAR T Cells Administered in Combination with Lymphodepletion and PD-1 Inhibition to Patients with Neuroblastoma. Molecular therapy: the journal of the American Society of Gene Therapy 25, 2214-2224, doi:10.1016/j.ymthe.2017.05.012 (2017).
18 Enblad, G. et al. A Phase I/IIa Trial Using CD19-Targeted Third-Generation CAR T Cells for Lymphoma and Leukemia. Clin Cancer Res 24, 6185-6194, doi:10.1158/1078-0432.Ccr-18-0426 (2018).
19 Pule, M. A. et al. Virus-specific T cells engineered to coexpress tumor-specific receptors: persistence and antitumor activity in individuals with neuroblastoma. Nat Med 14, 1264-1270, doi:10.1038/nm.1882 (2008).
20 Yu, A. L. et al. Anti-GD2 antibody with GM-CSF, interleukin-2, and isotretinoin for neuroblastoma. N Engl J Med 363, 1324-1334, doi:10.1056/NEJMoa0911123 (2010).
21 Du, H. et al. Antitumor Responses in the Absence of Toxicity in Solid Tumors by Targeting B7-H3 via Chimeric Antigen Receptor T Cells. Cancer Cell 35, 221-237.e228, doi:10.1016/j.ccell.2019.01.002 (2019).
22 Castelletti, L., Yeo, D., van Zandwijk, N. & Rasko, J. E. J. Anti-Mesothelin CAR T cell therapy for malignant mesothelioma. Biomark Res 9, 11, doi:10.1186/s40364-021-00264-1 (2021).
23 Landoni, E. et al. Modifications to the Framework Regions Eliminate Chimeric Antigen Receptor Tonic Signaling. Cancer Immunol Res, doi:10.1158/2326-6066.Cir-20-0451 (2021).
24 Beatty, G. L. et al. Mesothelin-specific chimeric antigen receptor mRNA-engineered T cells induce anti-tumor activity in solid malignancies. Cancer Immunol Res 2, 112-120, doi:10.1158/2326-6066.CIR-13-0170 (2014).
25 Fujiwara, K. et al. Hinge and Transmembrane Domains of Chimeric Antigen Receptor Regulate Receptor Expression and Signaling Threshold. Cells 9, doi:10.3390/cells9051182 (2020).
26 Chen, Y. et al. Eradication of Neuroblastoma by T Cells Redirected with an Optimized GD2-Specific Chimeric Antigen Receptor and Interleukin-15. Clin Cancer Res 25, 2915-2924, doi:10.1158/1078-0432.Ccr-18-1811 (2019).
27 Louis, C. U. et al. Antitumor activity and long-term fate of chimeric antigen receptor-positive T cells in patients with neuroblastoma. Blood 118, 6050-6056, doi:10.1182/blood-2011-05-354449 (2011).
28 Heczey, A. et al. Anti-GD2 CAR-NKT cells in patients with relapsed or refractory neuroblastoma: an interim analysis. Nat Med 26, 1686-1690, doi:10.1038/s41591-020-1074-2 (2020).
29 Kramer, K. et al. Disialoganglioside G(D2) loss following monoclonal antibody therapy is rare in neuroblastoma. Clin Cancer Res 4, 2135-2139 (1998).
30 Schumacher-Kuckelkorn, R. et al. Lack of immunocytological GD2 expression on neuroblastoma cells in bone marrow at diagnosis, during treatment, and at recurrence. Pediatr Blood Cancer 64, 46-56, doi:10.1002/pbc.26184 (2017).
31 Valitutti, S., Müller, S., Cella, M., Padovan, E. & Lanzavecchia, A. Serial triggering of many T-cell receptors by a few peptide-MHC complexes. Nature 375, 148-151, doi:10.1038/375148a0 (1995).
32 Kofler, D. M. et al. CD28 costimulation Impairs the efficacy of a redirected t-cell antitumor attack in the presence of regulatory t cells which can be overcome by preventing Lck activation. Molecular therapy: the journal of the American Society of Gene Therapy 19, 760-767, doi:10.1038/mt.2011.9 (2011).
33 Morello, A., Sadelain, M. & Adusumilli, P. S. Mesothelin-Targeted CARs: Driving T Cells to Solid Tumors. Cancer Discov 6, 133-146, doi:10.1158/2159-8290.Cd-15-0583 (2016).
34 Pellegatta, S. et al. Constitutive and TNFα-inducible expression of chondroitin sulfate proteoglycan 4 in glioblastoma and neurospheres: Implications for CAR-T cell therapy. Sci Transl Med 10, doi:10.1126/scitranslmed.aao2731 (2018).
35 Geldres, C. et al. T lymphocytes redirected against the chondroitin sulfate proteoglycan-4 control the growth of multiple solid tumors both in vitro and in vivo. Clin Cancer Res 20, 962-971, doi:10.1158/1078-0432.Ccr-13-2218 (2014).
36 Salter, A. I. et al. Phosphoproteomic analysis of chimeric antigen receptor signaling reveals kinetic and quantitative differences that affect cell function. Sci Signal 11, doi:10.1126/scisignal.aat6753 (2018).
37 Kawalekar, O. U. et al. Distinct Signaling of Coreceptors Regulates Specific Metabolism Pathways and Impacts Memory Development in CAR T Cells. Immunity 44, 380-390, doi:10.1016/j.immuni.2016.01.021 (2016).
38 Sohara, Y. et al. Lytic bone lesions in human neuroblastoma xenograft involve osteoclast recruitment and are inhibited by bisphosphonate. Cancer Res 63, 3026-3031 (2003).
39 Song, L. et al. Valpha24-invariant NKT cells mediate antitumor activity via killing of tumor-associated macrophages. J Clin Invest 119, 1524-1536, doi:10.1172/JC137869 (2009).
40 Song, L. et al. Oncogene MYCN regulates localization of NKT cells to the site of disease in neuroblastoma. J Clin Invest 117, 2702-2712, doi:10.1172/jci30751 (2007).
41 Hirschmann-Jax, C. et al. A distinct “side population” of cells with high drug efflux capacity in human tumor cells. Proc Natl Acad Sci USA 101, 14228-14233, doi:10.1073/pnas.0400067101 (2004).
42 Di Paolo, D. et al. Neuroblastoma-targeted nanoparticles entrapping siRNA specifically knockdown ALK. Molecular therapy: the journal of the American Society of Gene Therapy 19, 1131-1140, doi:10.1038/mt.2011.54 (2011).
43 Vera, J. et al. T lymphocytes redirected against the kappa light chain of human immunoglobulin efficiently kill mature B lymphocyte-derived malignant cells. Blood 108, 3890-3897, doi:10.1182/blood-2006-04-017061 (2006).
44 Diaconu, I. et al. Inducible Caspase-9 Selectively Modulates the Toxicities of CD19-Specific Chimeric Antigen Receptor-Modified T Cells. Molecular therapy: the journal of the American Society of Gene Therapy 25, 580-592, doi:10.1016/j.ymthe.2017.01.011 (2017).
45 Wang, G. et al. Fully human antibody V(H) domains to generate mono and bispecific CAR to target solid tumors. J Immunother Cancer 9, doi:10.1136/jitc-2020-002173 (2021).
46 Xu, Y. et al. Closely related T-memory stem cells correlate with in vivo expansion of CAR.CD19-T cells and are preserved by IL-7 and IL-15. Blood 123, 3750-3759, doi:10.1182/blood-2014-01-552174 (2014).
47 Imai, K., Wilson, B. S., Bigotti, A., Natali, P. G. & Ferrone, S. A 94,000-dalton glycoprotein expressed by human melanoma and carcinoma cells. J Natl Cancer Inst 68, 761-769 (1982).
48 Hoyos, V. et al. Engineering CD19-specific T lymphocytes with interleukin-15 and a suicide gene to enhance their anti-lymphoma/leukemia effects and safety. Leukemia 24, 1160-1170, doi:10.1038/leu.2010.75 (2010).
49 Love, M. I., Huber, W. & Anders, S. Moderated estimation of fold change and dispersion for RNA-seq data with DESeq2. Genome Biol 15, 550, doi:10.1186/s13059-014-0550-8 (2014).

1. A modified T cell comprising a dual targeting CAR, wherein the dual targeting CAR comprises:
a first CAR comprising a CD8α stalk and transmembrane domain and a CD28 intracellular domain, and
a second CAR comprising a CD8α stalk and transmembrane domain and a 4-1BB intracellular domain,
wherein the dual targeting CAR comprises split costimulatory signal and a single CAR-CD3ζ domain.

2. (canceled)

3. (canceled)

4. The modified T cell of claim 1, wherein the T cell exhibits dual antigen specificity and co-stimulation.

5. The modified T cell of claim 1, wherein the T cell exhibits killing activity and cytokine release of T cells via the first CAR or the second CAR.

6. The modified T cell of claim 1, wherein the T cell exhibits increased IFN-γ and IL-2 release, as compared to a control cell.

7. The modified T cell of claim 1, wherein the T cell exhibits higher basal levels of TCR activation signaling, as compared to a control cell.

8. The modified T cell of claim 1, wherein the T cell exhibits enhanced phosphorylation of the CAR-CD3ζ chain and downstream signaling kinases.

9. (canceled)

10. The modified T cell of claim 1, wherein the T cell exhibits enrichment in cell cycle pathways.

11. The modified T cell of claim 1, wherein the T cell exhibits enrichment in TCR signaling pathways.

12. The modified T cell of claim 1, wherein the T cell exhibits elevated glycolytic activity, as compared to a control cell.

13. The modified T cell of claim 1, wherein the T cell controls tumor growth upon tumor re-challenge, as compared to a control cell.

14. The modified T cell of claim 1, wherein the T cell promotes enhanced tumor control and improved survival, as compared to a control cell.

15. The modified T cell of claim 1, wherein the T cell exhibits increased anti-tumor activity.

16. The modified T cell of claim 15, wherein the T cell exhibits increased anti-tumor activity under stress conditions.

17. The modified T cell of claim 1, wherein the T cell is a human T cell.

18. The modified T cell of claim 1, wherein the T cell is a non-human T cell.

19. (canceled)

20. A population of the modified T cells of claim 1, wherein the modified T cells are CD4+ and CD8+ T cells.

21. The population of claim 20, wherein the modified T cells are enriched in CD27+/CD29+cells in both the CD4+ and CD8+ T cells.

22. A modified T cell comprising GD2.28ζCAR/B7-H3.BB.CAR.

23. A method of treating cancer comprising administering to a subject a modified T cell comprising a dual targeting CAR, wherein the dual targeting CAR comprises:
a first CAR comprising a CD8α stalk and transmembrane domain and a CD28 intracellular domain, and
a second CAR comprising a CD8α stalk and transmembrane domain and a 4-1BB intracellular domain,
wherein the dual targeting CAR comprises split costimulatory signal and a single CAR-CD3ζ domain.

24.-30. (canceled)

31. The modified T cell of claim 1, where the first CAR and the second CAR form a dimer through the CD8α stalk.
